(19) United States
(12) Patent Application Publication
(10) Pub. No.: US 2013/0158264 A1
(43) Pub. Date: Jun. 20, 2013

(54) Polymorphs of OSI-906

(71) Applicants: Arlindo L. Castelhano, New City, NY (US); David A. Enger, West Lafayette, IN (US); Jason A. Hanko, West Lafayette, IN (US); Josef A. Rechka, Port Jefferson, NY (US); Jing Teng, West Lafayette, IN (US); Yonglai Yang, West Lafayette, IN (US)

(72) Inventors: Arlindo L. Castelhano, New City, NY (US); David A. Enger, West Lafayette, IN (US); Jason A. Hanko, West Lafayette, IN (US); Josef A. Rechka, Port Jefferson, NY (US); Jing Teng, West Lafayette, IN (US); Yonglai Yang, West Lafayette, IN (US)

(21) Appl. No.: 13/805,402

(22) PCT Filed: Jun. 23, 2011
(86) PCT No.: PCT/US11/41547

§ 371 Date: Mar. 6, 2013

Related U.S. Application Data
(60) Provisional application No. 61/357,688, filed on Jun. 23, 2010.

(51) Int. Cl. C07D 487/04

(52) U.S. Cl. CPC C07D 487/04
USPC 544/350

(57) ABSTRACT

Polymorphic forms of the tyrosine kinase inhibitor OSI-906, preparation, pharmaceutical compositions, and uses thereof. The invention includes methods of treating diseases such as cancer, including cancer mediated at least in part by IGF-1 R and/or IR, with the polymorphs and compositions. This Abstract is not limiting of the invention.

This application claims the benefit and priority of U.S. Appl. No. 61/357,688, filed Jun. 23, 2010, which is incorporated herein in its entirety by this reference.

### BACKGROUND OF THE INVENTION

The present invention pertains at least in part to cancer treatment, certain chemical compounds, and methods of treating tumors and cancers with the compounds.

The development of target-based anti-cancer therapies has become the focus of a large number of pharmaceutical research and development programs. Various strategies of intervention include targeting protein tyrosine kinases, including receptor tyrosine kinases believed to drive or mediate tumor growth.

Insulin-like growth factor-1 receptor (IGF-1R) is a receptor tyrosine kinase that plays a key role in tumor cell proliferation and apoptosis inhibition, and has become an attractive cancer therapy target. IGF-1R is involved in the establishment and maintenance of cellular transformation, is frequently overexpressed by human tumors, and activation or overexpression thereof mediates aspects of the malignant phenotype. IGF-1R activation increases invasion and metastasis propensity.

Inhibition of receptor activation has been an attractive method having the potential to block IGF-mediated signal transduction. Anti-IGF-1R antibodies to block the extracellular ligand-binding portion of the receptor and small molecules to target the enzyme activity of the tyrosine kinase domain have been developed. See Expert Opin. Ther. Patents, 17(1):25-35 (2007); Expert Opin. Ther. Targets, 12(5):589-603 (2008); and Am J. Transl. Res., 1:101-114 (2009).

US 2006/0235031 (published Oct. 19, 2006) describes a class of bicyclic ring substituted protein kinase inhibitors, including Example 31 thereof, which corresponds to the dual IR/IGF-1R inhibitor known as OSI-906. As of 2011, OSI-906 is in clinical development in various cancers and tumor types. The preparation and characterization of OSI-906, which can be named as cis-3-[8-amino-1-(2-phenyl-quinolin-7-yl)-imidazo[1,5-a]pyrazin-3-yl]-1-methylcyclobutanol, is described in the aforementioned US 2006/0235031.

OSI-906 is a potent, selective, and orally bioavailable dual IGF-1R/IR kinase inhibitor with favorable drug-like properties. The selectivity profile of OSI-906 in conjunction with its ability to inhibit both IGF-1R and IR affords the special opportunity to fully target the IGF-1R/IR axis. See Future Med. Chem., 1(6), 1153-1171, (2009).

New polymorphic forms can provide various advantages, including reproducibility for use in pharmaceutical formulations, and improved physical characteristics such as stability, solubility, bioavailability, or processability/handling characteristics. Polymorphic forms are prepared and tested to better understand the relative physiochemical properties of a given drug. Identification of the most promising form(s) can be essential for successful product development. For example, the most thermodynamically stable form can be selected for development. See Wiley Series in Drug Discovery and Development, Evaluation of Drug Candidates for Preclinical Development: Pharmacokinetics, Metabolism, Pharmaceutics, and Toxicology, 1-281, (2010).

Regulatory agencies may require definitive control of polymorphic form of drug substances. Therefore, novel polymorphic forms of OSI-906 with improved and controllable physical properties are desired.

### SUMMARY OF THE INVENTION

In some aspects, the invention provides polymorphic forms of OSI-906 (cis-3-[8-amino-1-(2-phenyl-quinolin-7-yl)-imidazo[1,5-a]pyrazin-3-yl]-1-methylcyclobutanol).

In certain aspects, the invention provides polymorphic hydrate forms of OSI-906.

In certain aspects, the invention provides polymorphic solvate forms of OSI-906.

In certain aspects, the invention provides polymorphic unsolvated forms of OSI-906.

In certain aspects, the invention provides polymorph Form A, which was identified as an unsolvated crystalline form of OSI-906.

In additional aspects the invention provides Form B, which was identified as most likely being a monohydrate crystalline form of OSI-906.

In additional aspects the invention provides Form C, which was identified as a hemihydrate or variable hydrate crystalline form of OSI-906.

In additional aspects the invention provides Form D, which was identified as a monohydrate crystalline form of OSI-906.

In additional aspects the invention provides Form E, which was identified as a possible hemihydrate crystalline form of OSI-906.

In additional aspects the invention provides Form F, which was identified as a isopropanol solvate crystalline form of OSI-906.

In additional aspects the invention provides Form G, which was identified as a nitromethane solvate crystalline form of OSI-906.

In additional aspects the invention provides Form H, which was identified as a acetonitrile solvate crystalline form of OSI-906.

The invention provides methods of preparing and isolating polymorphic forms including forms A-H of OSI-906. The invention provides pharmaceutical compositions of OSI-906 polymorphic Forms A-H. The invention provides for methods of treating disease such as cancer and conditions for which treatment with an IGF-1R/IR inhibitor is effective, with OSI-906 Forms A-H. The invention provides for the use of the polymorphs of OSI-906 in the manufacture of a medicament for such treatment.

### BRIEF DESCRIPTION OF THE DRAWINGS

FIG. 1: Overlayed XRPD patterns of OSI-906 Forms A-G.

FIG. 2: XRPD pattern of OSI-906 Form A.

FIG. 3: XRPD pattern of OSI-906 Form B.

FIG. 4: XRPD pattern of OSI-906 Form C.

FIG. 5: XRPD pattern of OSI-906 Form D.

FIG. 6: XRPD pattern of OSI-906 Form E.

FIG. 7: XRPD pattern of OSI-906 Form F.

FIG. 8: XRPD pattern of OSI-906 Form G.

FIG. 9: XRPD pattern of OSI-906 Form H.

FIG. 10: FTIR spectrum of OSI-906 Form A.

FIG. 11: FTIR spectrum of OSI-906 Form B.

FIG. 12: FTIR spectrum of OSI-906 Form C.

FIG. 13: FTIR spectrum of OSI-906 Form D.

FIG. 14: FTIR spectrum of OSI-906 Form E.

FIG. 15: FTIR spectrum of OSI-906 Form F.

FIG. 16: DSC thermogram of OSI-906 Form A.

FIG. 17: TGA profile of OSI-906 Form A.

FIG. 18: DSC thermogram of OSI-906 Form B.

FIG. 19: TGA profile of OSI-906 Form B.

FIG. 20: DSC thermogram of OSI-906 Form C.

FIG. 21: TGA profile of OSI-906 Form C.

FIG. 22: DSC thermogram of OSI-906 Form D.

FIG. 23: TGA profile of OSI-906 Form D.

FIG. 24: DSC thermogram of OSI-906 Form E.

FIG. 25: TGA profile of OSI-906 Form E.

FIG. 26: DSC thermogram of OSI-906 Form F.

FIG. 27: TGA profile of OSI-906 Form F.

FIG. 28: DSC thermogram of OSI-906 Form G.

FIG. 29: TGA profile of OSI-906 Form G.

FIG. 30: ¹H NMR spectrum (in DMSO-d₆) of OSI-906 Form A.

FIG. 31: Overlay of ¹H NMR spectrum (in DMSO-d₆) of OSI-906 Form B (top) and Form A (bottom).

FIG. 32: Overlay of ¹H NMR spectrum (in DMSO-d₆) of OSI-906 Form C (top) and Form A (bottom).

FIG. 33: Overlay of ¹H NMR spectrum (in DMSO-d₆) of OSI-906 Form D (top) and Form A (bottom).

FIG. 34: Overlay of ¹H NMR spectrum (in DMSO-d₆) of OSI-906 Form E (top) and Form A (bottom).

FIG. 35: Overlay of ¹H NMR spectrum (in DMSO-d₆) of OSI-906 Form F (top) and Form A (bottom).

FIG. 36: Overlay of ¹H NMR spectrum (in DMSO-d₆) of OSI-906 Form G (top) and Form A (bottom).

FIG. 37: Oak Ridge Thermal Ellipsoid Plot (ORTEP) drawing of OSI-906. Atoms are represented by 50% probability anisotropic thermal ellipsoids.

FIG. 38: Gravimetric Moisture Sorption curve of Form A.

FIG. 39: Stack plot of XRPD patterns of OSI-906 solid forms (from top): (a) Form A; (b) following moisture sorption analysis of Form A; (c) 7 days of storage under desiccant conditions; (d) 7 days of storage at 25° C./60% RH; (e) 7 days of storage at 40° C./75% RH.

FIG. 40: Stack plot of XRPD patterns of OSI-906 solid forms (from top): (a) Form A; (b) 7 days of storage at 40° C. under vacuum; (c) 7 days of storage at 80° C. under vacuum; (d) After mortar and pestle grinding, 7 days of storage at 80° C. under vacuum; (e) After ball mill grinding, 7 days of storage at 80° C. under vacuum.

FIG. 41: Stack plot of ¹H-NMR spectra of OSI-906 solid forms (from top): (a) Form A; (b) 7 days of storage at 40° C. under vacuum; (c) 7 days of storage at 80° C. under vacuum.

FIG. 42: XRPD pattern of OSI-906 Form F obtained from single solvent crystallization in IPA.

FIG. 43: Stack Plot of XRPD patterns of OSI-906 IPA solvate (Form F) (from top): (a) Form F; (b) Mixture of Forms C and F obtained following 8 days of storage of Form F in a sealed vial at ambient temperature; (c) Form C.

FIG. 44: Linear regression for calibration and validation samples with Form D.

FIG. 45: FTIR spectra of OSI-906 Forms A and F; (unique adsorption bands ↓ signature of Form F not observed in Form A.

FIG. 46: Raman spectra of OSI-906 Forms A and F; (unique adsorption bands ↓ signature of Form F not observed in Form A.

FIG. 47: Gravimetric Moisture Sorption Curve of Form C.

FIG. 48: Stack plot of XRPD patterns of OSI-906 solid forms (from top): (a) Form C; (b) following moisture sorption analysis of Form C resulting in a mixture of Forms C+I; (c) Form I following overnight storage of Form C under desiccant conditions; (d) Form C obtained following 1 hour of exposure of Form Ito lab humidity, 40-50% RH; (e) following DSC isothermal hold of Form C at 105° C. for five minutes.

FIG. 49: DSC thermogram of OSI-906 Form C.

FIG. 50: DSC thermograms of OSI-906 Form C: (a) DSC scan from 30-300° C. at 10° C./min; (b) DSC scan from 30-105° C. at 10° C./min following isothermal hold at 105° C. for 5 minutes; (c) Sample exposed to lab humidity overnight following isothermal hold at 105° C. for 5 minutes.

FIG. 51: TGA thermogram of OSI-906 Form C.

FIG. 52: Stack plot of XRPD patterns of OSI-906 solid forms (from top): (a) Forms C+D; (b) following 7 days of Form C+D storage under desiccant conditions; (c) following 7 days of Form C+D storage at 25° C./60% RH; (d) following 7 days of Form C+D storage at 40° C./75% RH; (e) following 7 days of Form C+D storage at 40° C. under vacuum affording Form C; (f) following 7 days of Form C+D storage at 80° C. under vacuum affording Form C; (g) Form D.

FIG. 53: Stack plot of XRPD patterns of OSI-906 solid forms (from top): (a) Form C; (b) Form D; (c) Form I; (d) following 3 days of Form C+D storage under desiccant conditions affording a mixture of Forms C+D+I.

FIG. 54: Stack plot of XRPD patterns of OSI-906 solid forms (from top): (a) Following 11 day room temperature slurry of Form C in THF affording Form A; (c) Following 11 day room temperature slurry of Forms A+C+D in IPA affording Form A; (d) Following 5 day 50° C. slurry of Forms C+D in EtOH affording a mixture of Forms A and E.

FIG. 55: Gravimetric Moisture Sorption Curve of Form D.

FIG. 56: Stack plot of XRPD patterns of OSI-906 solid forms (from top): (a) Form D; (b) following moisture sorption analysis of Form D resulting in a mixture of Forms C and D; (c) Form C.

FIG. 57: Stack plot of XRPD patterns of OSI-906 solid forms (from top): (a) Form A; (b) 11 day room temperature slurry in THF affording Form A; (c) 5 day 50° C. slurry in DI water affording Form A; (d) 7 day 50° C. slurry in DI water affording Form D; (e) 11 day room temperature slurry in EtOH affording Form C.

FIG. 58: Stack plot of XRPD patterns of OSI-906 solid forms (from top): (a) Form A; (b) following 5 day slurry of Forms C+D in THF at 50° C. affording Form A; (c) following 11 day room temperature slurry of Forms A+C+D in IPA affording Form A.

FIG. 59: Stack plot of XRPD patterns of OSI-906 solid forms (from top): (a) Form A; (b) Form C; (c) following 5 day slurry of Forms C+D in EtOH at 50° C. affording Forms A+E; (d) following 11 day room temperature slurry of Forms C+D in EtOH affording Form C; (e) following 11 day room temperature slurry of Forms C+D in (80:20) EtOH:Water affording Form C.

FIG. 60: Representative Raman Spectra of OSI-906 Forms A, C, and D.

FIG. 61: Linear regression for calibration sample of Form C.

FIG. 62: Linear regression for calibration sample of Form D.

FIG. 63: Linear regression for calibration and validation samples with Form C.

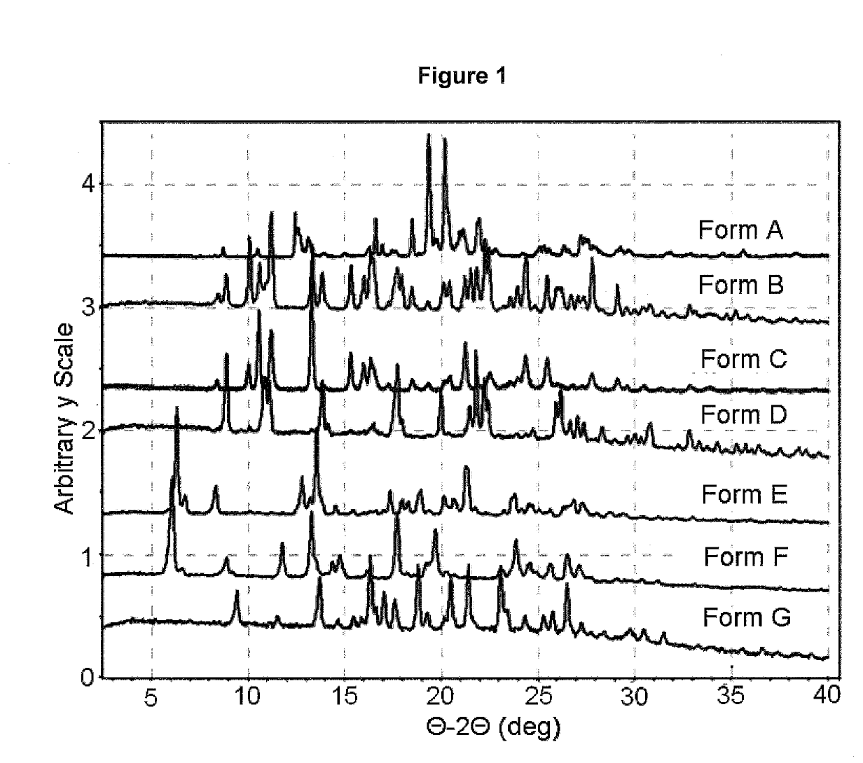

### DETAILED DESCRIPTION OF THE INVENTION

The present invention concerns polymorphic forms of Formula I, as shown below and defined herein:

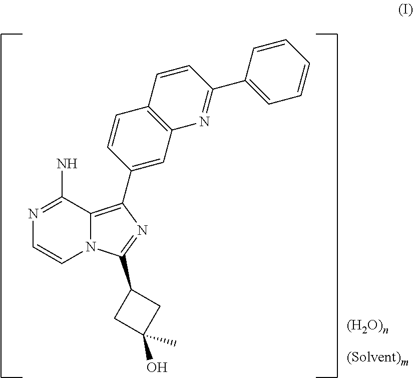

wherein, n and m are independently 0, 0.5, 1, or 2 and the term “solvent” is a suitable organic solvent such as but not limited to an alcohol or a polar solvent.

The present invention includes Formula I, wherein the solvent is a suitable organic solvent such as but not limited to methanol, ethanol, isopropanol, n-propanol, n-butanol, sec-butanol, t-butanol, iso-butanol, acetonitrile, and nitromethane.

The present invention further concerns polymorphic forms of Formula II, as shown below and defined herein:

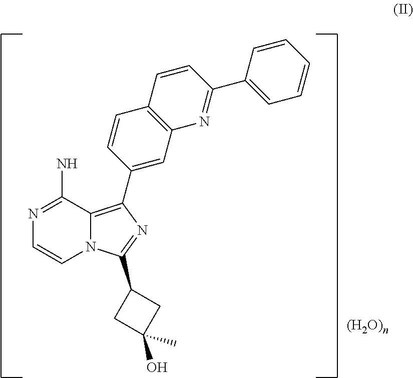

wherein, n is 0, 0.5, 1 or 2.

The present invention concerns polymorphic forms of Formula III, as shown below and defined herein:

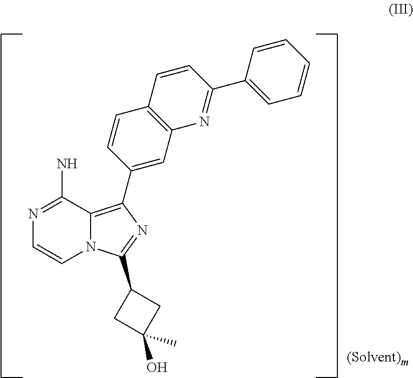

wherein, m is 0, 1 or 2 and the term “solvent” is a suitable organic solvent such as but not limited to an alcohol or a polar solvent.

In some aspects, the present invention provides crystalline polymorph Form A of OSI-906.

In some aspects thereof, the polymorph Form A exhibits an X-ray diffraction pattern comprising peaks (°2θ) at about 12.4, 12.6, 16.6, 18.5, 19.4, 20.2, and 22; in some aspects, the polymorph is present as a material comprising at least about 95% by weight Form A based on the total amount of OSI-906; is present as a material comprising at least about 98% by weight Form A based on the total amount of OSI-906; is present as a material that is substantially free of amorphous OSI-906, OSI-906 hydrates, and OSI-906 solvates; or is substantially free of solvent.

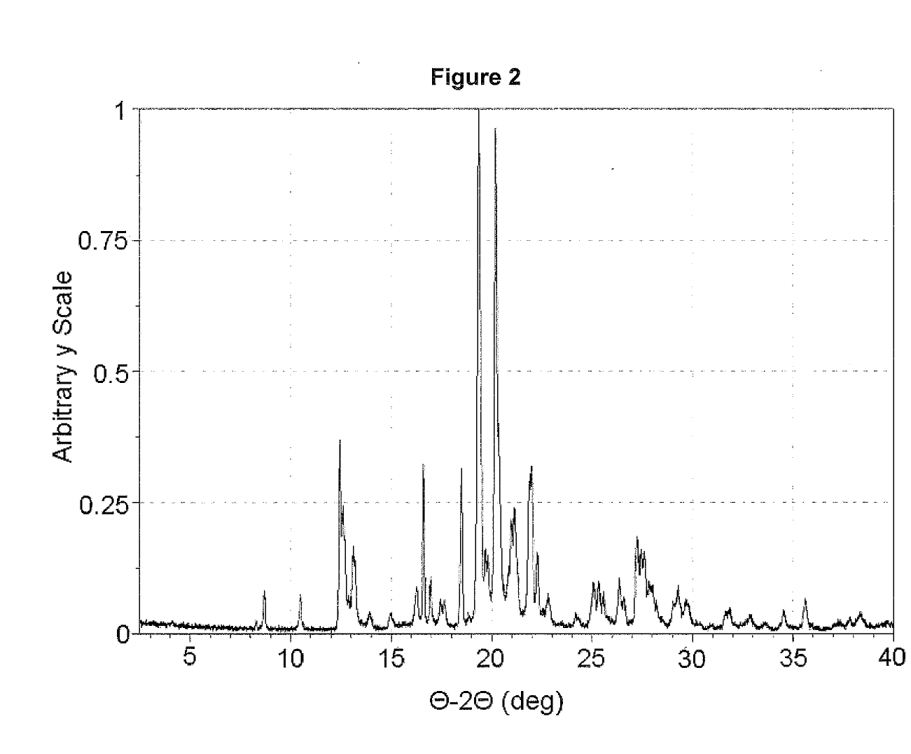

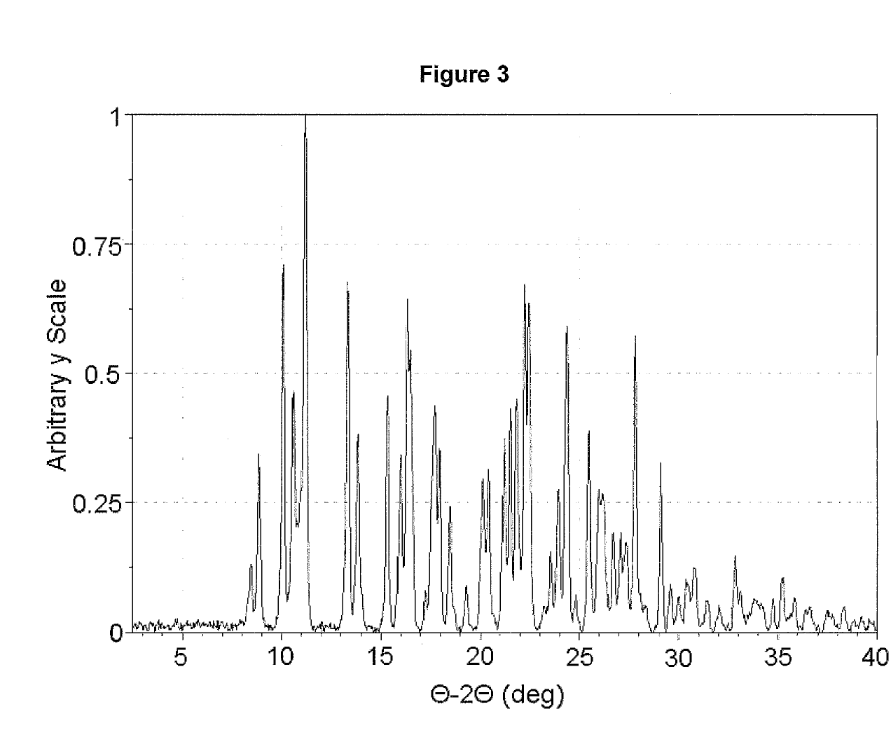

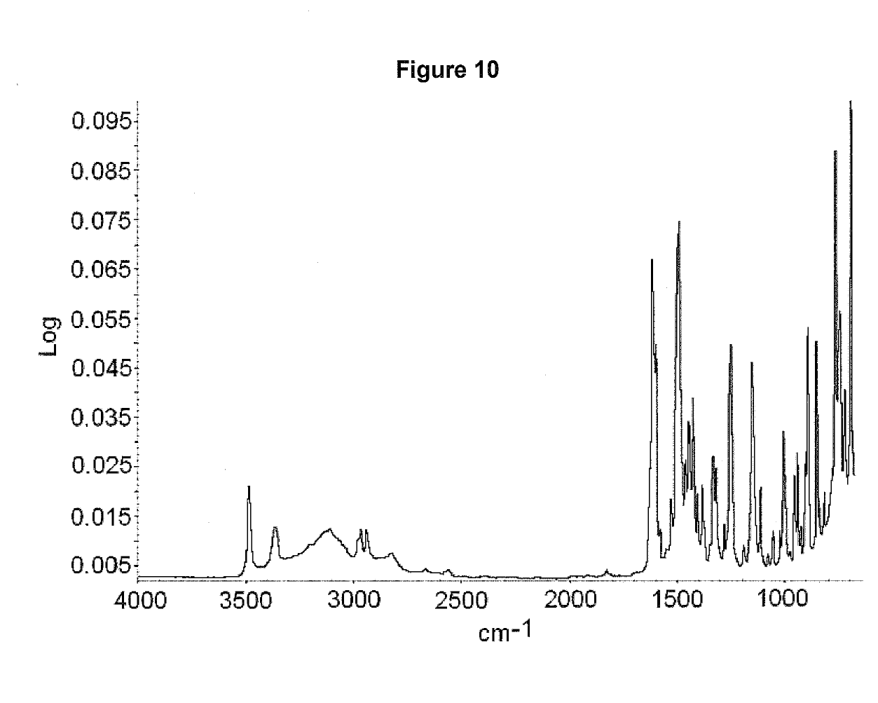

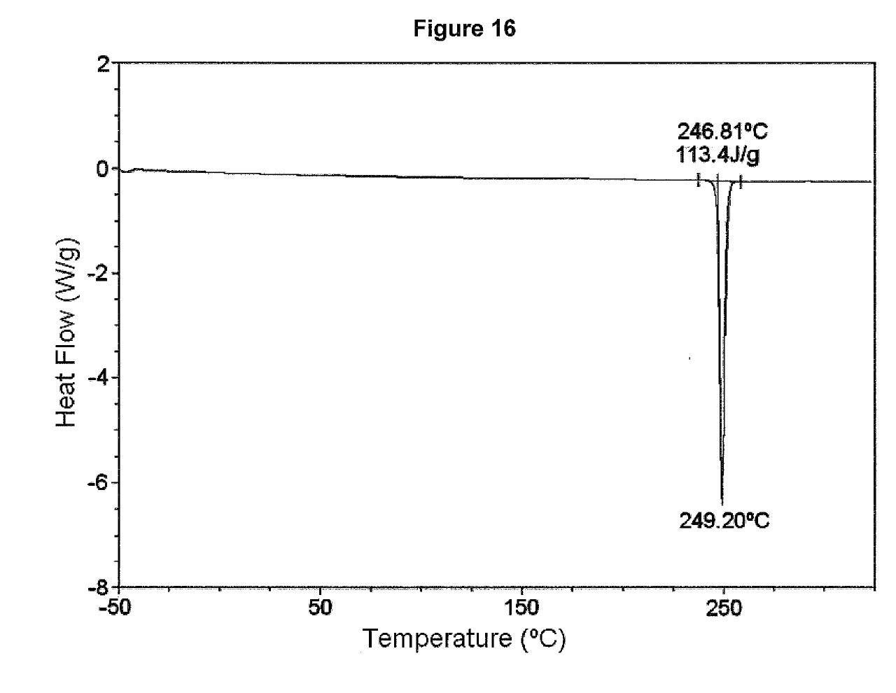

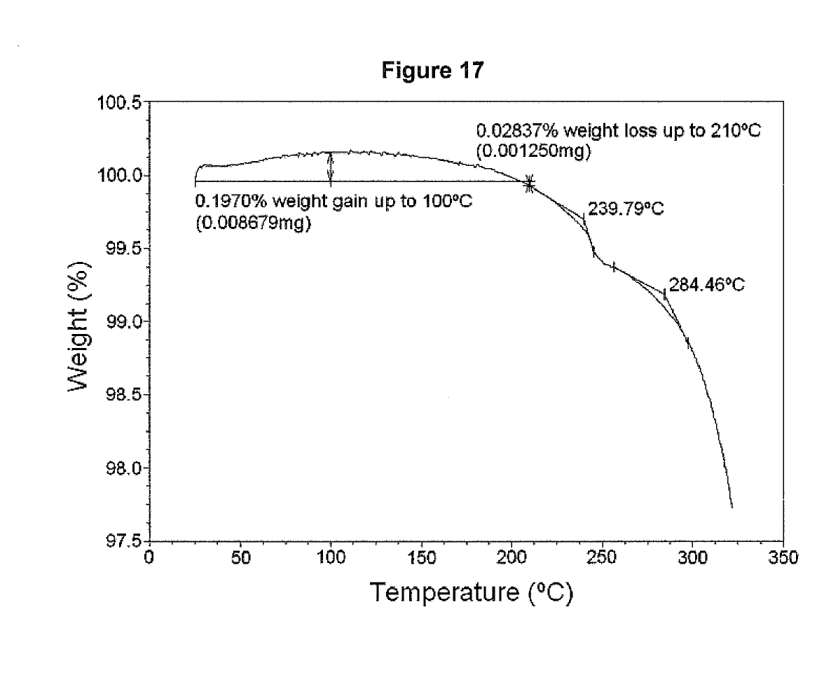

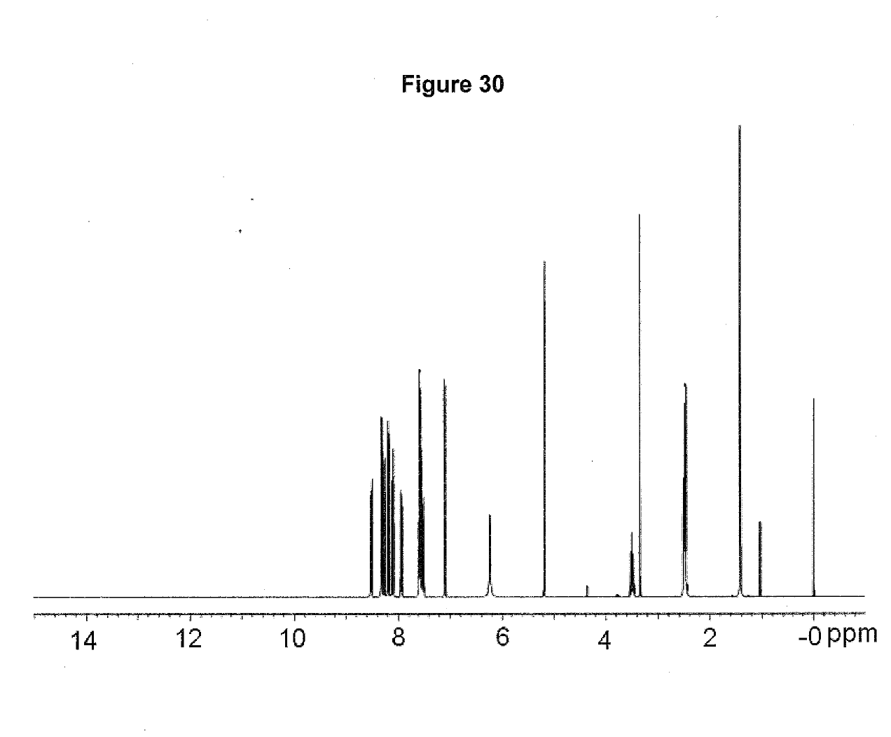

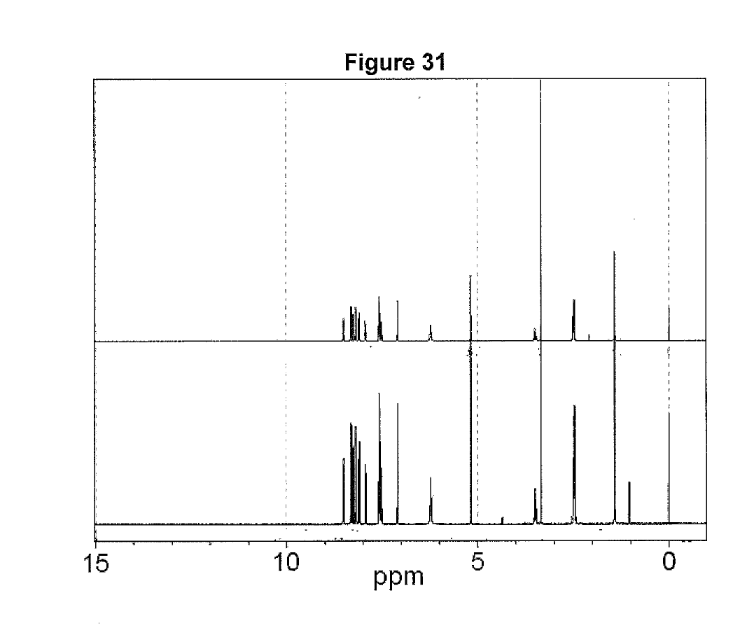

In some aspects, there is provided crystalline polymorph Form A, which exhibits one or more of an X-ray diffraction pattern with characteristic peaks substantially as set forth in Table 1, an X-ray diffraction pattern substantially resembling that of FIG. 2, a DSC thermogram substantially resembling that of FIG. 16, a TGA signal substantially resembling that of FIG. 17, an IR spectrum substantially resembling that of FIG. 10, or a ¹H NMR spectrum in DMSO-d₆ substantially resembling that of FIG. 30.

In some aspects, there is provided crystalline polymorph Form A, which is present as a material comprising at least about 50% to 98% or more by weight Form A based on the total amount of OSI-906. In some aspects, the Form A is present as a material comprising at least about 95% or about 98% by weight Form A based on the total amount of OSI-906.

In some aspects, there is provided crystalline polymorph Form A, which is present as a material that is substantially free of amorphous OSI-906 and substantially free hydrates or solvates of OSI-906.

In some aspects, there is provided crystalline polymorph Form A of OSI-906, which is prepared by a process comprising: (a) preparing a slurry of OSI-906 in an alcohol; (b) heating the slurry; and (c) isolating crystalline Form A such as by filtration.

In some aspects, there is provided crystalline polymorph Form A of OSI-906, which is prepared by a process comprising: (1) dissolving OSI-906 in water at acidic pH of about 3, (2) raising the pH to precipitate the product such as pH about 5, (3) isolating the product such as by filtration, (4) suspending the product in an alcohol such as IPA to give a slurry, and (5) isolating and drying resulting Form A.

In further aspects, the preparing a slurry in (a) further comprises adjusting pH to about 5. In further aspects, the preparing a slurry in further comprises agitating the slurry at ambient temperature. In further aspects, the heating in comprises heating to about 60° C. to 90° C., or about 75-85° C. In further aspects, the isolating crystalline Form A in comprises washing the crystalline Form A with an alcohol. In further aspects, the isolating crystalline Form A further comprises filtering crystalline Form A and drying crystalline Form A under vacuum. In further aspects, the alcohol comprises isopropanol, n-propanol, n-butanol, sec-butanol, t-butanol, or iso-butanol. In some aspects, the alcohol is isopropanol (IPA).

The present invention further provides for crystalline polymorph Form B of OSI-906.

In some aspects, the polymorph Form B exhibits an X-ray diffraction pattern comprising peaks (°2θ) at about 10.1, 10.6, 11.2, 13.3, 15.3, 16.3, 21.8, 22.3, 22.4, 24.4, and 27.8.

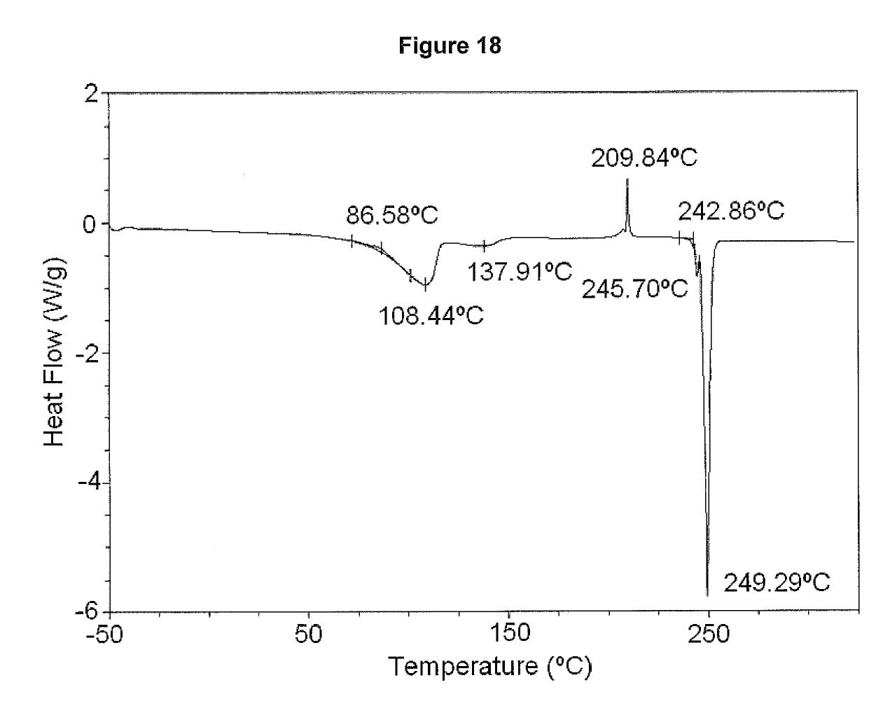

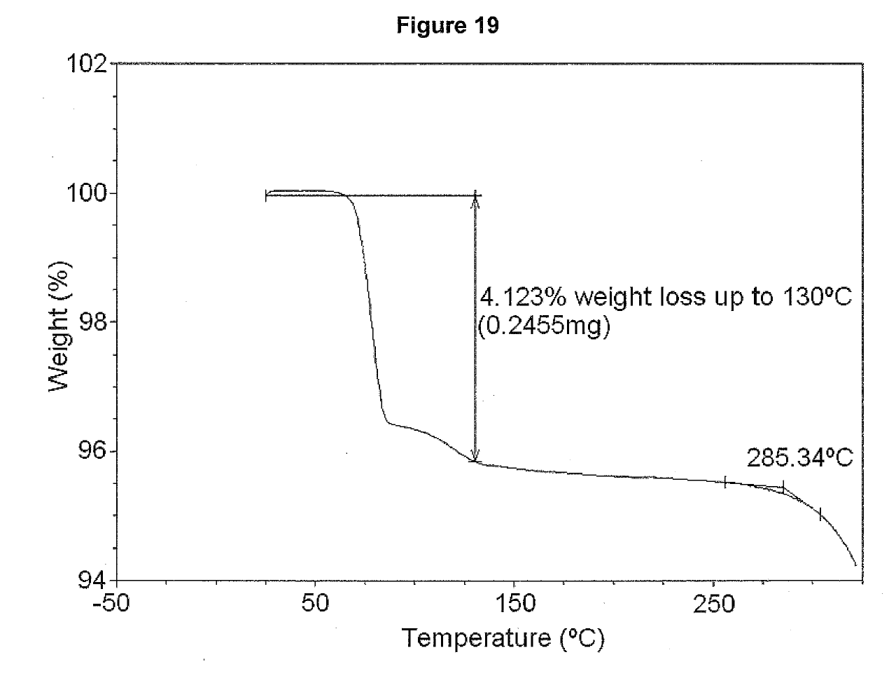

In some aspects, polymorph Form B exhibits one or more of an X-ray diffraction pattern with characteristic peaks as set forth in Table 3, an X-ray diffraction pattern substantially resembling that of FIG. 3, a DSC thermogram substantially resembling that of FIG. 18, a TGA signal substantially resembling that of FIG. 19, or a ¹H NMR spectrum in DMSO-d₆ substantially resembling that of FIG. 31.

In some aspects, there is provided crystalline polymorph Form B, which is present as a material that is about 50% to 98% or more by weight Form B based on the total amount of OSI-906. In some aspects, the Form B is present as a material comprising at least about 95% or about 98% by weight Form B based on the total amount of OSI-906.

In some aspects, there is provided crystalline polymorph Form B, which is present as a material that is substantially free of amorphous OSI-906.

In some aspects, there is provided crystalline polymorph Form B, which is present as a material that is substantially free of OSI-906 other than polymorph Form B.

In some aspects, there is provided crystalline polymorph Form B, which is prepared by a process comprising: (a) preparing a slurry of OSI-906 in a polar solvent and water such as CH₃CN:water (e.g., 60:40); and (b) isolating crystalline Form B.

In further aspects, the preparing a slurry in (a) further comprises sonicating the slurry. In further aspects, the preparing a slurry in (a) further comprises agitating the slurry, e.g., at ambient temp., e.g., for about 4 days. In further aspects, the slurry is seeded with Form B. In further aspects, the isolating crystalline Form B in (b) further comprises filtering crystalline Form B and drying crystalline Form B under vacuum. In further aspects, the polar solvent in (a) comprises acetonitrile. In some embodiments, a solution of OSI-906 is prepared prior to preparing the slurry.

The present invention further provides for crystalline polymorph Form C of OSI-906.

In some aspects, polymorph Form C exhibits an X-ray diffraction pattern comprising peaks (°2θ) at about 10.6, 11.2, 13.3, 15.3, 21.2, 24.3, and 25.5.

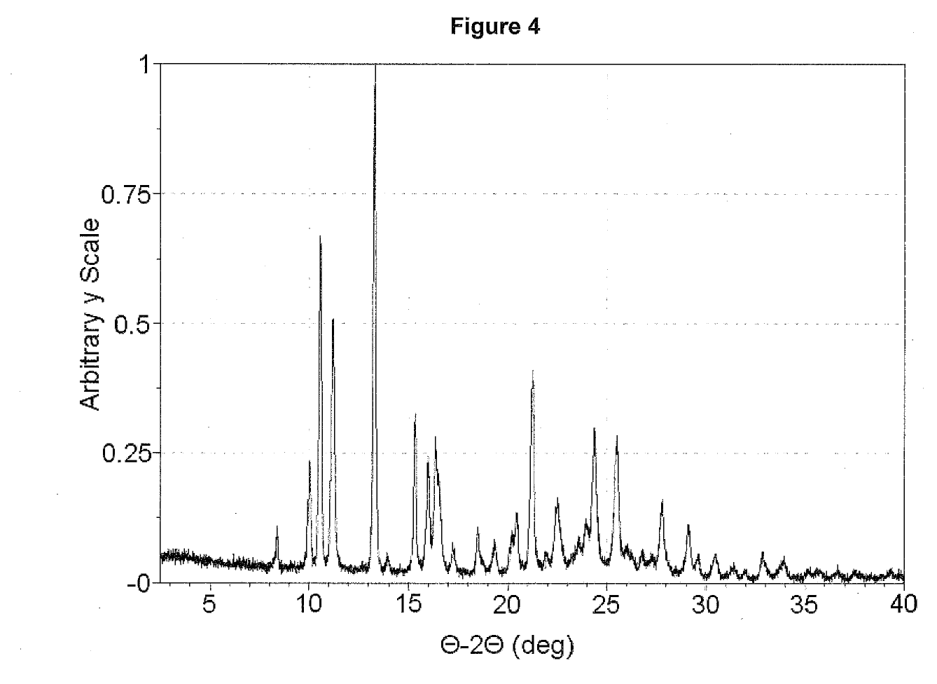

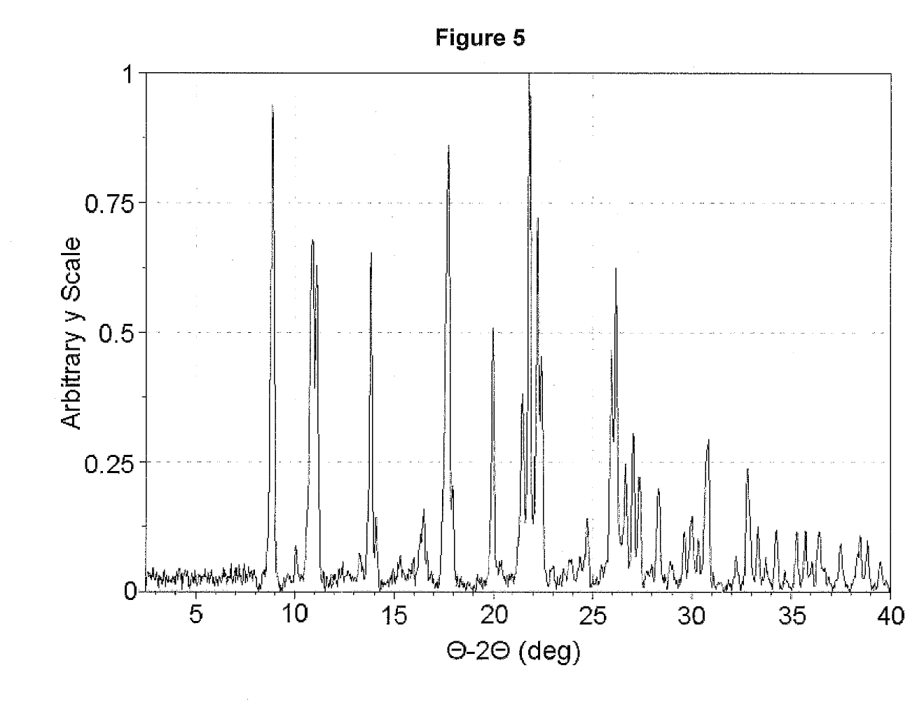

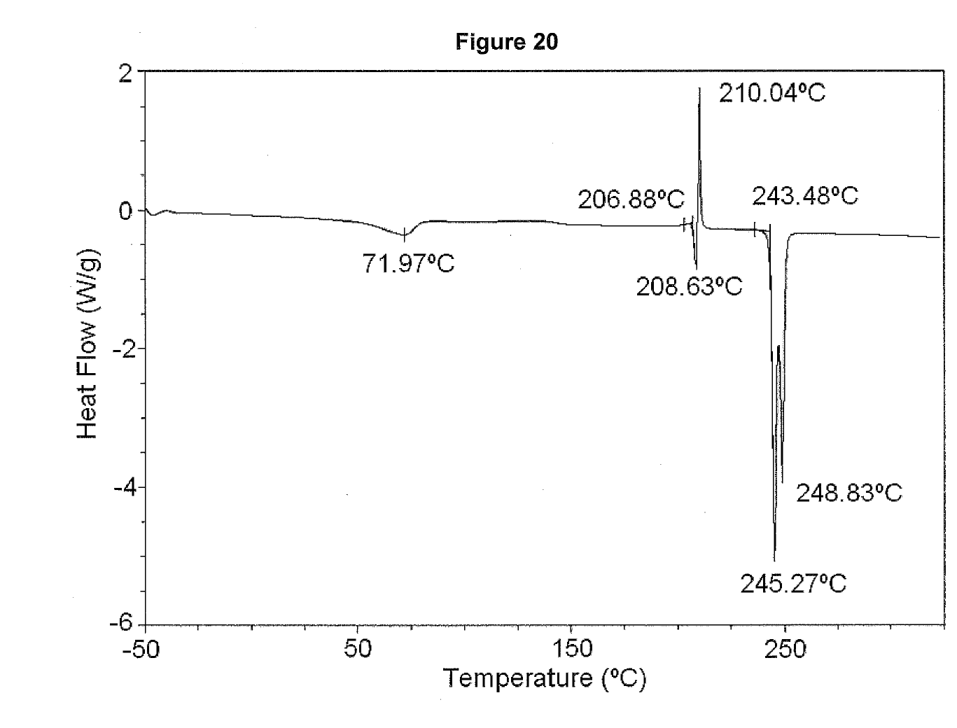

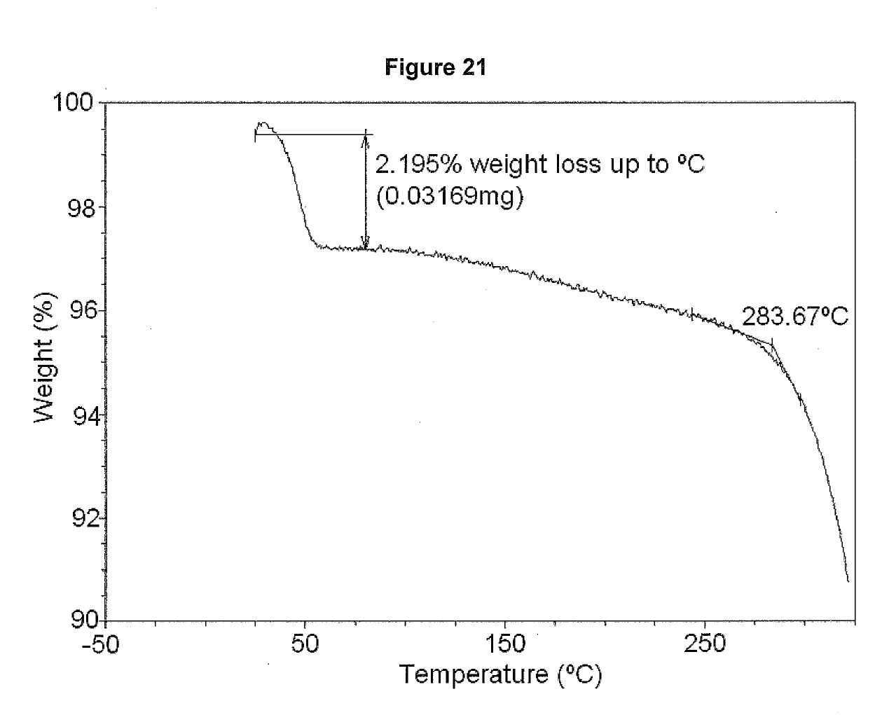

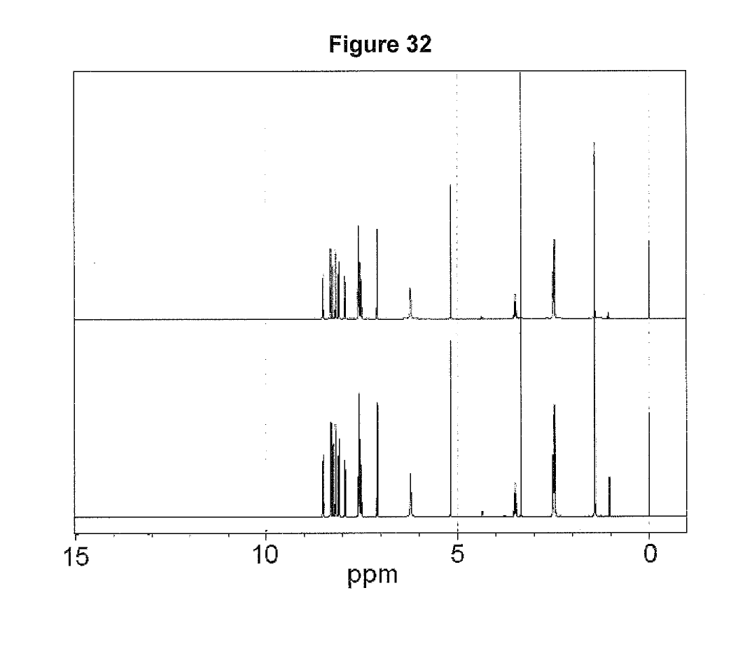

In some aspects, polymorph Form C exhibits one or more of an X-ray diffraction pattern with characteristic peaks as set forth in Table 5, an X-ray diffraction pattern substantially resembling that of FIG. 4, a DSC thermogram substantially resembling that of FIG. 20, a TGA signal substantially resembling that of FIG. 21, or a ¹H NMR spectrum in DMSO-d₆ substantially resembling that of FIG. 32.

In some aspects, there is provided crystalline polymorph Form C, which is present as a material comprising about 50% to 98% or more by weight Form C based on the total amount of OSI-906. In some aspects, the Form C is present as a material comprising at least about 95% or about 98% or more by weight Form C based on the total amount of OSI-906.

In some aspects, there is provided crystalline polymorph Form C, which is present as a material that is substantially free of amorphous OSI-906 and substantially free of hydrates or solvates of OSI-906 other than polymorph Form C.

In some aspects, there is provided crystalline polymorph Form C, which is prepared by a process comprising: (a) preparing a solution of OSI-906 in an alcohol; (b) heating the solution; and (c) isolating crystalline Form C. In further aspects, the preparing a solution in (a) further comprises sonicating the solution.

In further aspects, the heating in (b) further comprises heating to about 60° C. to 90° C., or about 65 to 75° C. and/or agitating. In further aspects, the isolating crystalline Form C in (c) further comprises filtering the solution of Form C into a container within a cooling bath. In further aspects, the cooling bath is about −0° C. to −20° C. In further aspects, the solution of Form C is cooled in a freezer. In further aspects, the isolating crystalline Form C in (c) further comprises filtering crystalline Form C and drying crystalline Form C under vacuum. In further aspects, the alcohol in (a) comprises methanol, ethanol, isopropanol, n-propanol, n-butanol, sec-butanol, or iso-butanol. In some embodiments, the alcohol is ethanol.

The present invention further provides for crystalline polymorph Form D of OSI-906.

In some aspects, polymorph Form D exhibits an X-ray diffraction pattern comprising peaks (°2θ) at about 8.9, 10.9, 11.1, 13.8, 17.7, 20, 21.8, 22.2, and 26.2.

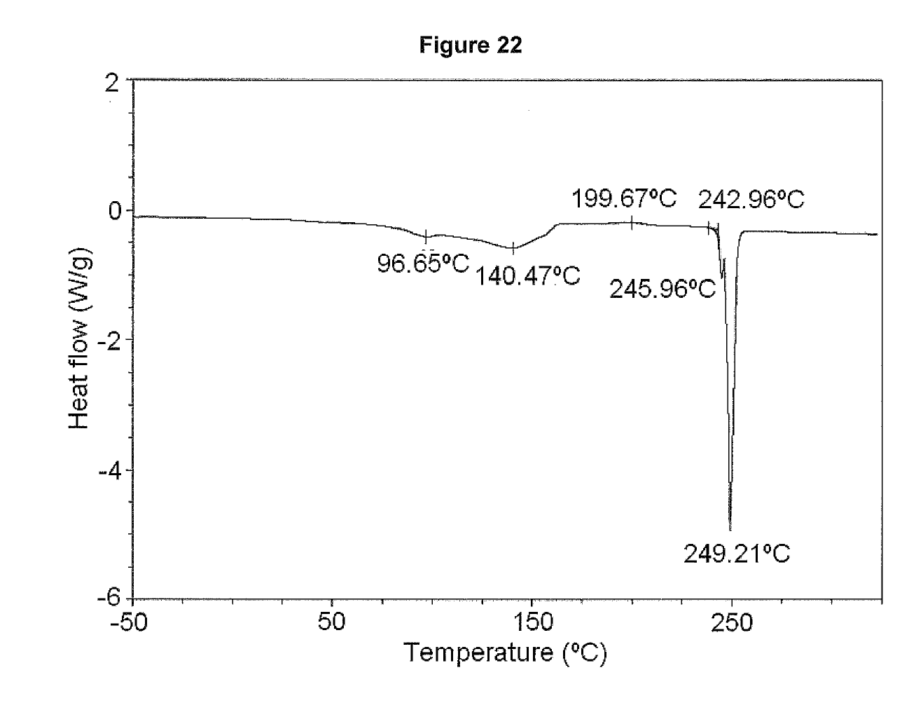

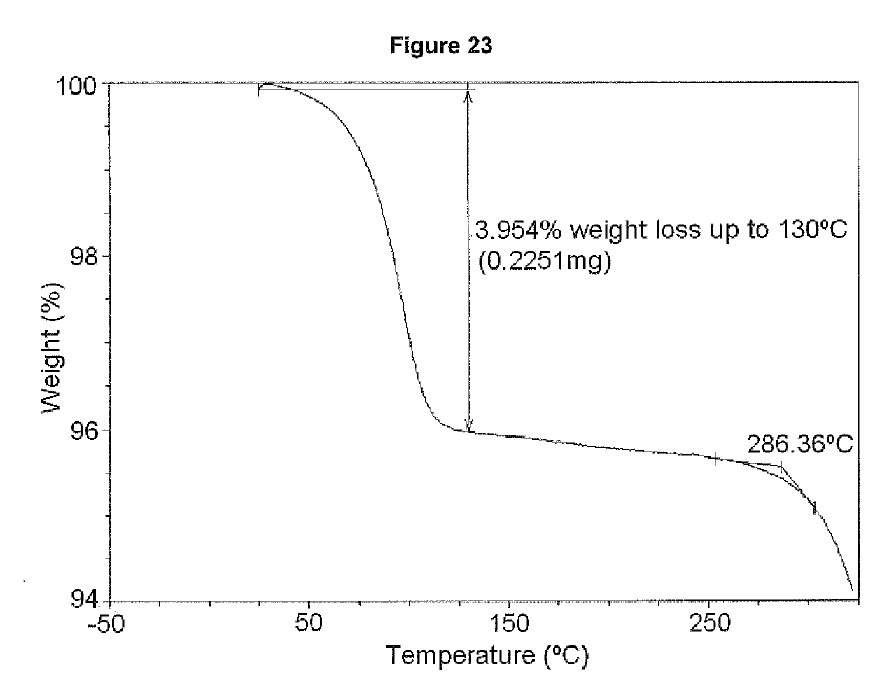

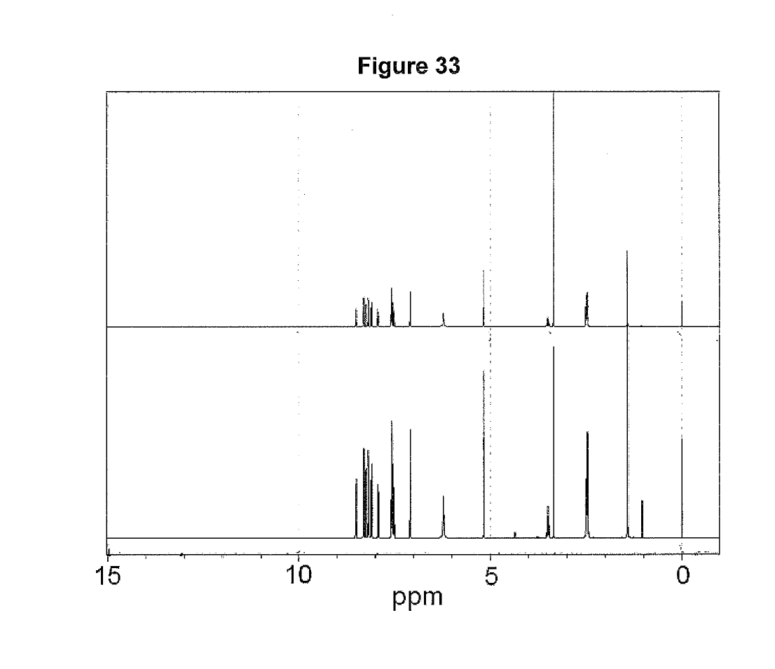

In some aspects, there is provided crystalline polymorph Form D, which exhibits one or more of an X-ray diffraction pattern with characteristic peaks as set forth in Table 7, an X-ray diffraction pattern substantially resembling that of FIG. 5, a DSC thermogram substantially resembling that of FIG. 22, a TGA signal substantially resembling that of FIG. 23, or a ¹H NMR spectrum in DMSO-d₆ substantially resembling that of FIG. 33.

In some aspects, there is provided crystalline polymorph Form D, which is present as a material that is about 50% to 98% or more by weight Form D based on the total amount of OSI-906. In some aspects, the Form D is present as a material comprising at least about 95% or about 98% or more by weight Form D based on the total amount of OSI-906.

In some aspects, there is provided crystalline polymorph Form D, which is present as a material that is substantially free of amorphous OSI-906.

In some aspects, there is provided crystalline polymorph Form D, which is present as a material that is substantially free of OSI-906 other than polymorph Form D.

In some aspects, there is provided crystalline polymorph Form D, which is prepared by a process comprising: (a) preparing a slurry of OSI-906 in an aqueous alcohol; (b) heating the slurry; and (c) isolating crystalline Form D. In further aspects, the preparing a slurry in (a) further comprises 60:40 (v/v) ethanol:water. In further aspects, the preparing a slurry in (a) further comprises agitating solution. In further aspects, the heating in (b) further comprises heating to about 50° C. to 90° C. In further aspects, the heating in (b) further comprises agitating the slurry. In further aspects, the isolating crystalline Form D in (c) further comprises seeding the slurry with Form D. In further aspects the isolating crystalline Form D in (c) further comprises filtering crystalline Form D and drying crystalline Form D under vacuum. In further aspects, the alcohol in (a) comprises methanol, ethanol, isopropanol, n-propanol, n-butanol, sec-butanol, or iso-butanol.

The present invention further provides crystalline polymorph Form E of OSI-906.

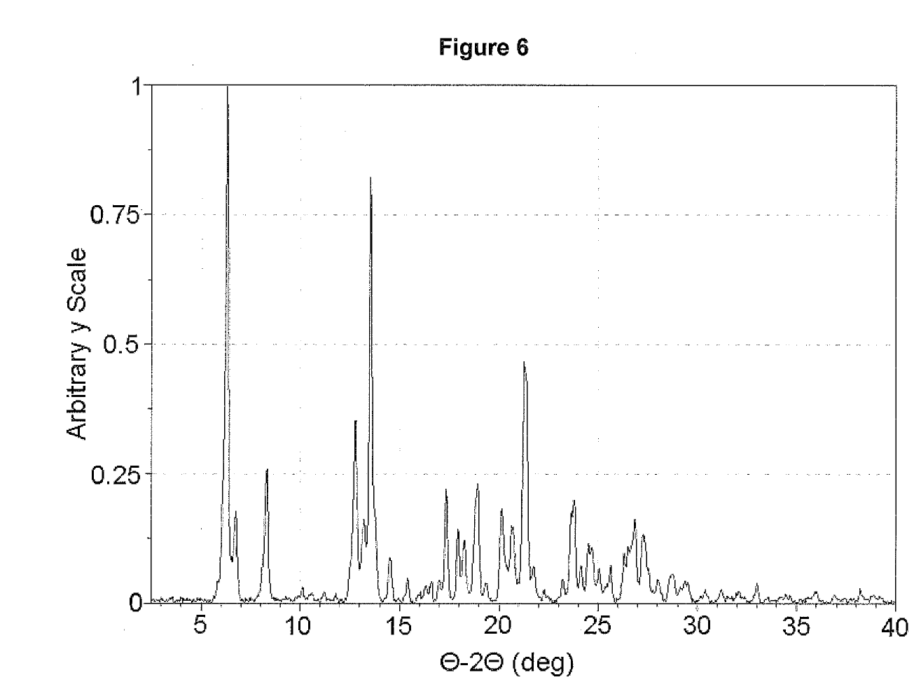

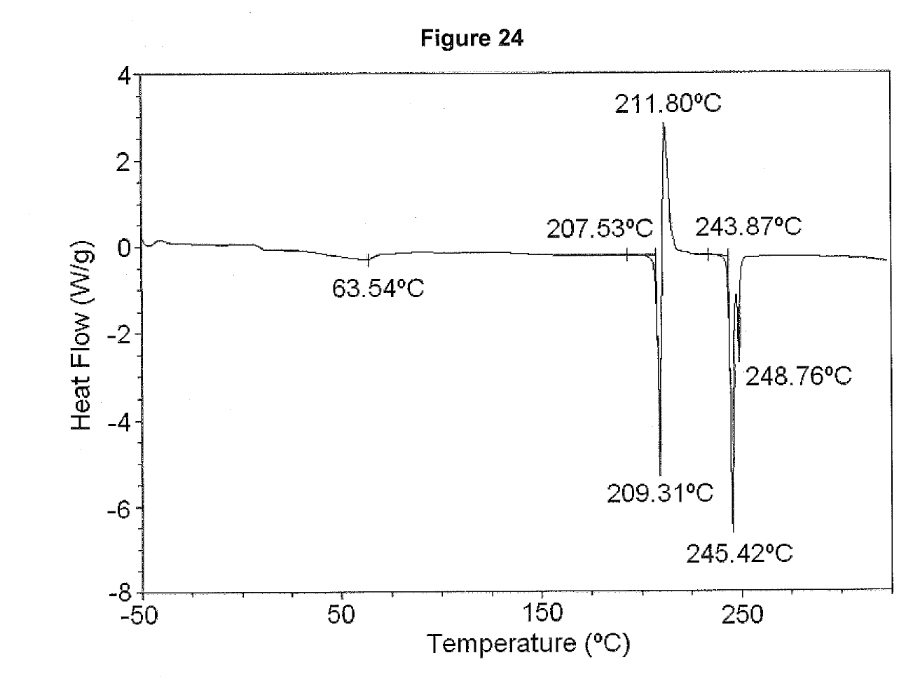

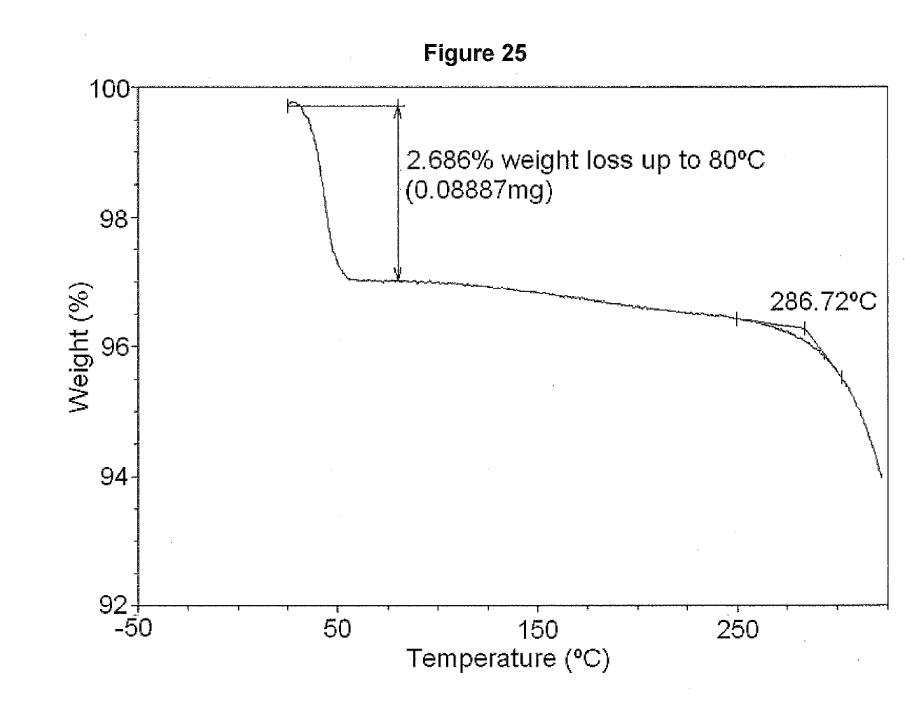

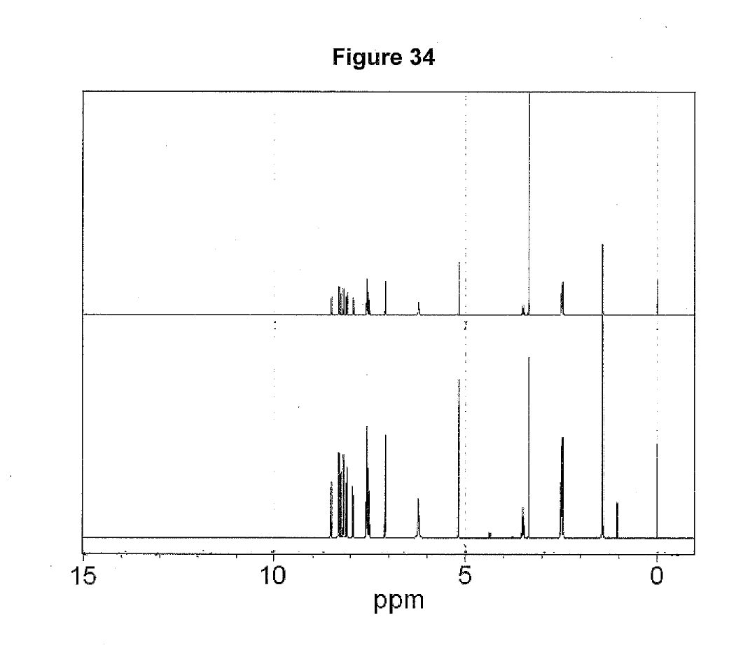

In some aspects, there is provided crystalline polymorph Form E, which exhibits one or more of an X-ray diffraction pattern with characteristic peaks as set forth in Table 9, an X-ray diffraction pattern substantially resembling that of FIG. 6, a DSC thermogram substantially resembling that of FIG. 24, a TGA signal substantially resembling that of FIG. 25, or a ¹H NMR spectrum in DMSO-d₆ substantially resembling that of FIG. 34.

In some aspects, there is provided crystalline polymorph Form E, which is present as a material that is at least about 50% or 98% or more by weight Form E based on the total amount of OSI-906.

In some aspects, there is provided crystalline polymorph Form E, which is present as a material that is substantially free of amorphous OSI-906.

In some aspects, there is provided crystalline polymorph Form E, which is present as a material that is substantially free of OSI-906 other than polymorph Form E.

In some aspects, there is provided crystalline polymorph Form E, which is prepared by a process comprising: (a) preparing a slurry of OSI-906 in an alcohol; (b) heating the slurry; and (b) isolating crystalline Form E. In further aspects, the preparing a slurry in (a) further comprises sonicating slurry. In further aspects, the heating in (b) further comprises heating to about 60° C. to 90° C. In further aspects, the heating in (b) further comprises agitating the slurry. In further aspects, the isolating crystalline Form E in (c) further comprises filtering and cooling the slurry to about −0° C. to −20° C. In further aspects, the isolating crystalline Form E in (c) further comprises seeding the slurry with Form C. In further aspects, the isolating crystalline Form E in (c) further comprises filtering crystalline Form E and drying crystalline Form E under vacuum. In further aspects, the alcohol in (a) comprises methanol, ethanol, isopropanol, n-propanol, n-butanol, sec-butanol, or iso-butanol.

The present invention further provides for crystalline polymorph Form F of OSI-906.

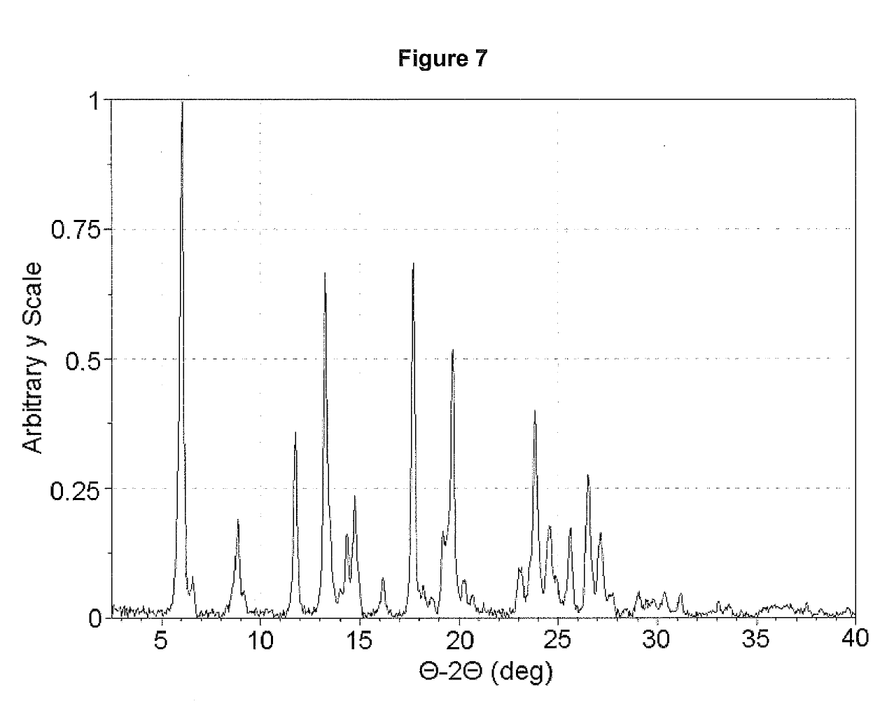

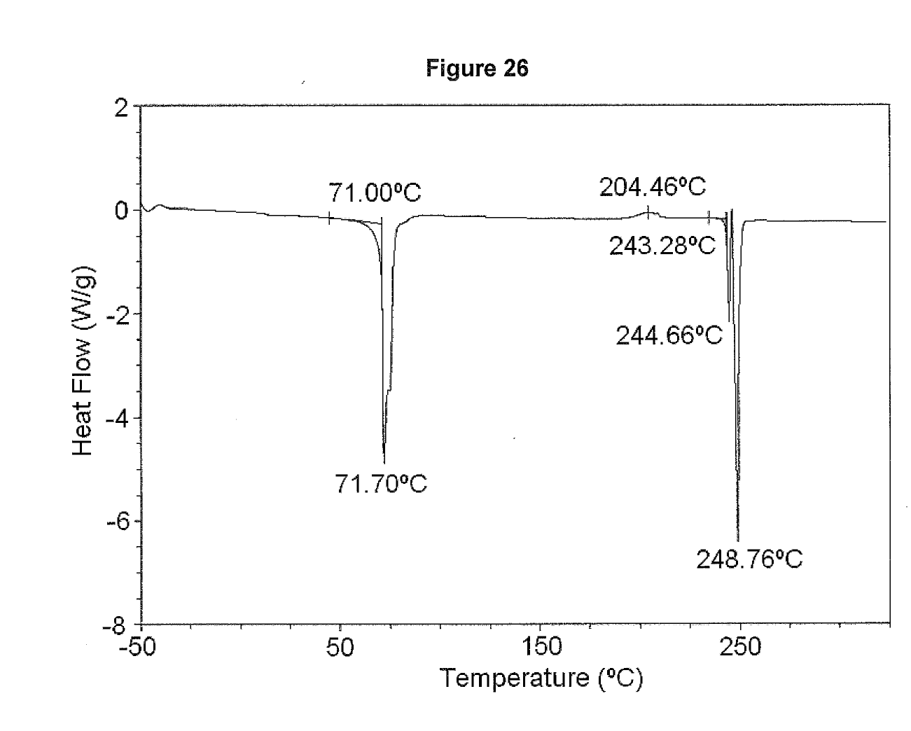

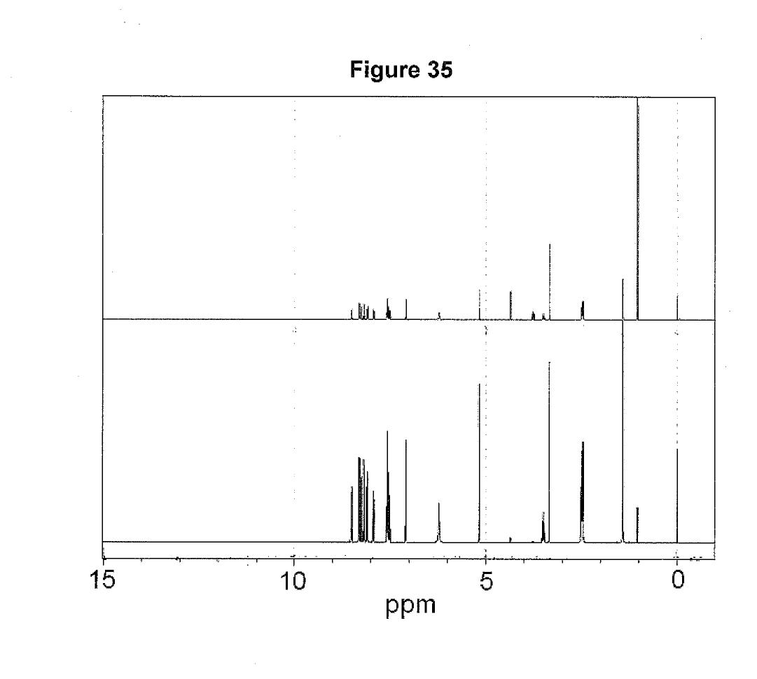

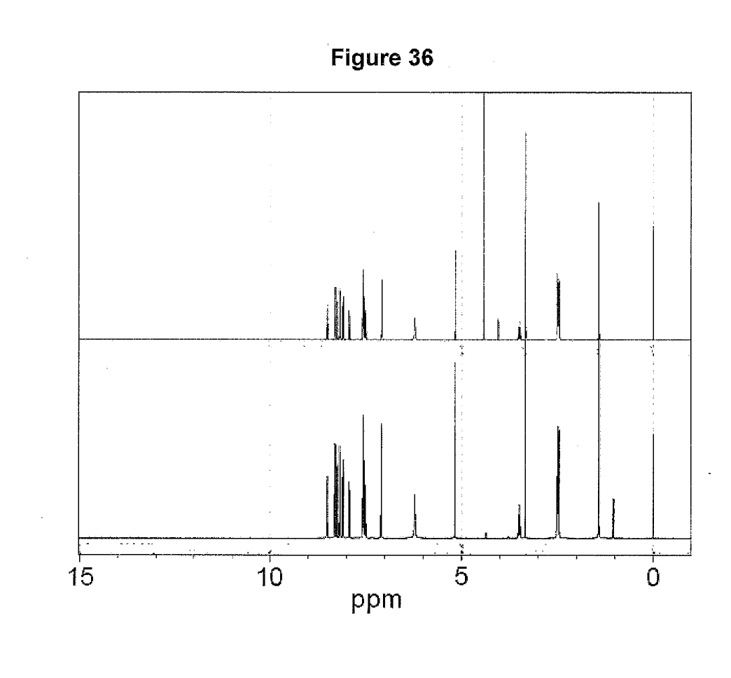

In some aspects, there is provided crystalline polymorph Form F, which exhibits one or more of an X-ray diffraction pattern with characteristic peaks as set forth in Table 11, an X-ray diffraction pattern substantially resembling that of FIG. 7, a DSC thermogram substantially resembling that of FIG. 25, a TGA signal substantially resembling that of FIG. 26, or a ¹H NMR spectrum in DMSO-d₆ substantially resembling that of FIG. 35.

In some aspects, there is provided crystalline polymorph Form F, which is present as a material that is at least about 50% or about 98% or more by weight Form F based on the total amount of OSI-906.

In some aspects, there is provided crystalline polymorph Form F, which is present as a material that is substantially free of amorphous OSI-906.

In some aspects, there is provided crystalline polymorph Form F, which is present as a material that is substantially free of OSI-906 other than polymorph Form F.

In some aspects, there is provided crystalline polymorph Form F, which is prepared by a process comprising: (a) preparing a solution of OSI-906 in isopropanol; (b) heating the solution; and (c) isolating crystalline Form F.

In further aspects, the preparing a solution in (a) further comprises agitating the solution. In further aspects, the heating in (b) further comprises heating to about 60° C. to 90° C. In further aspects, the isolating crystalline Form F in (c) further comprises filtering, cooling solution to ambient and then to about −0° C. to −20° C. In further aspects, the isolating crystalline Form F in (c) further comprises seeding the solution with Form F. In further aspects, there the isolating crystalline Form F in (c) further comprises filtering crystalline Form F and drying crystalline Form F under vacuum.

The present invention further provides for crystalline polymorph Form G of OSI-906.

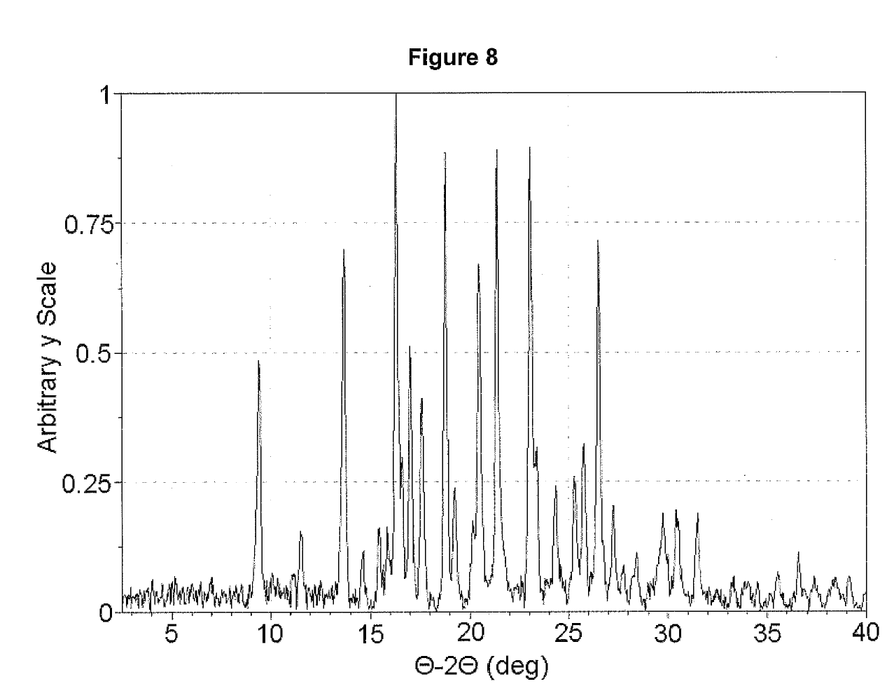

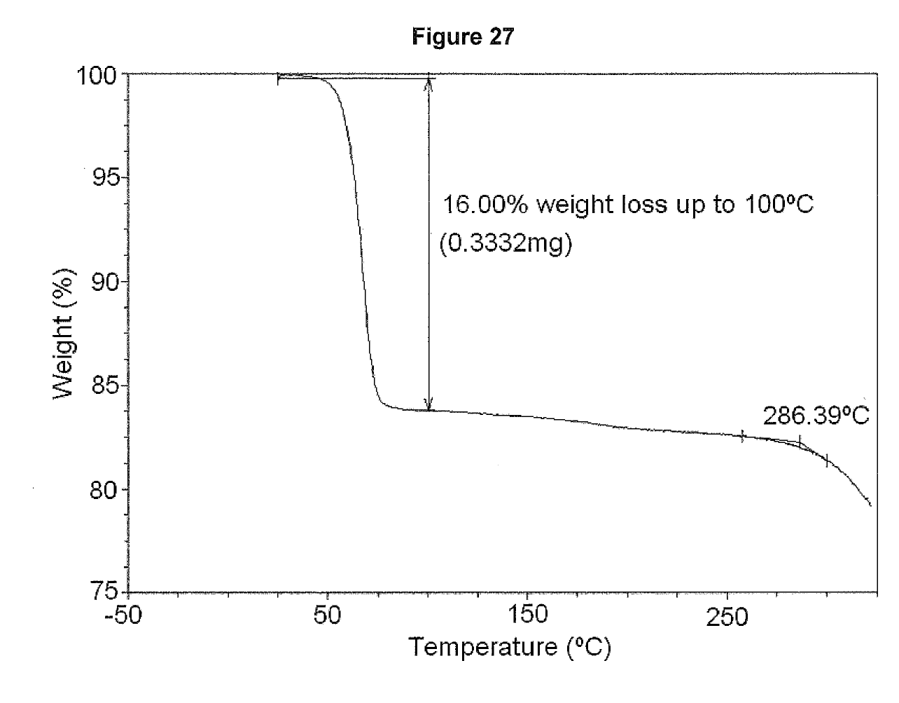

In some aspects, there is provided crystalline polymorph Form G, which exhibits one or more of an X-ray diffraction pattern with characteristic peaks as set forth in Table 13, an X-ray diffraction pattern substantially resembling that of FIG. 8, a DSC thermogram substantially resembling that of FIG. 26, a TGA signal substantially resembling that of FIG. 27, or a ¹H NMR spectrum in DMSO-d₆ substantially resembling that of FIG. 36.

In some aspects, there is provided crystalline polymorph Form G, which is present as a material that is at least about 50% or about 98% or more by weight Form G based on the total amount of OSI-906.

In some aspects, there is provided crystalline polymorph Form G, which is present as a material that is substantially free of amorphous OSI-906.

In some aspects, there is provided crystalline polymorph Form G, which is present as a material that is substantially free of OSI-906 other than polymorph Form G.

In some aspects, there is provided crystalline polymorph Form G, which is prepared by a process comprising: (a) preparing a solution of OSI-906 in nitromethane; (b) heating the solution; and (c) isolating crystalline Form G. In further aspects, the heating in (b) further comprises agitating the solution. In further aspects, the isolating crystalline Form G in (c) further comprises filtering, cooling solution to ambient and then to about −0° C. to −20° C. In further aspects, the isolating crystalline Form G in (b) further comprises seeding the solution with Form G. In further aspects, the isolating crystalline Form G in (b) further comprises filtering crystalline Form G and drying crystalline Form G under vacuum.

The present invention further provides for crystalline polymorph Form H of OSI-906.

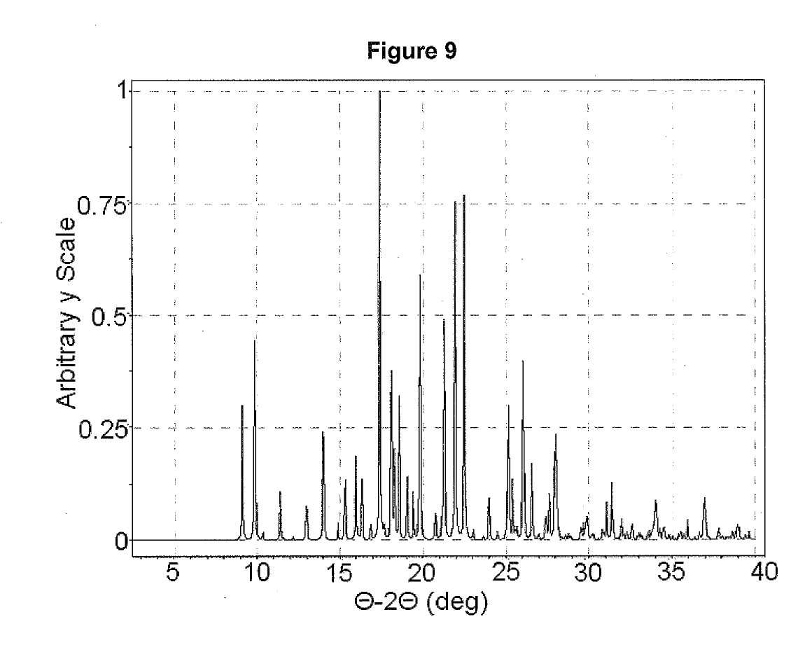

In some aspects, there is provided crystalline polymorph Form H, which exhibits an X-ray diffraction pattern substantially resembling that of FIG. 9 and an X-ray single crystal diffraction pattern as set forth in Tables 16-20.

In some aspects, there is provided crystalline polymorph Form H, which is present as a material that is at least about 50% or about 98% or more by weight Form H based on the total amount of OSI-906.

In some aspects, there is provided crystalline polymorph Form H, which is present as a material that is substantially free of amorphous OSI-906.

In some aspects, there is provided crystalline polymorph Form H, which is present as a material that is substantially free of OSI-906 other than polymorph Form H.

In some aspects, there is provided crystalline polymorph Form H, which is prepared by a process comprising: (a) preparing a slurry of OSI-906 in acetonitrile; and (b) isolating crystalline Form H.

In some aspects, there is provided crystalline polymorph Form H, which is prepared by a process comprising: (a) preparing a solution of OSI-906 in nitromethane; (b) evaporating the nitromethane; and (b) isolating crystalline Form H.

In further aspects, the preparing a slurry in (a) further comprises sonicating the slurry. In further aspects, the preparing a slurry in (a) further comprises agitating the slurry at ambient for 4 days. In further aspects, the isolating crystalline Form H in (b) further comprises filtering crystalline Form H and drying crystalline Form H under vacuum.

### EXPERIMENTAL

### Instrumental Techniques (Preparation and Characterization—Forms A-I)

Identification of the crystalline forms obtained by the present invention can be made by methods known in the art, including but not limited to X-Ray powder diffraction (XRPD), Fourier Transform Infrared (FTIR) spectra, and Differential Scanning calorimetry (DSC), Thermogravimetric Analysis (TGA), Nuclear Magnetic Resonance (NMR), and single crystal X-ray diffraction. Furthermore, it should be understood that operator, instrument and other related changes may result in some margin of error with respect to analytical characterization of the crystalline forms.

Differential Scanning Calorimetry (DSC):

Analyses were carried out on a TA Instruments differential scanning calorimeter 2920. The instrument was calibrated using indium as the reference material. The sample was placed into a standard aluminum DSC pan, and the weight accurately recorded. The sample cell was equilibrated at −50° C. and heated under a nitrogen purge at a rate of 10° C./min, up to a final temperature of 325° C. To determine the glass transition temperature (T_{g}) of amorphous material, the sample cell was heated starting from ambient under a nitrogen purge at a rate of 10° C./min, up to 260° C., hold 1 min at 260° C.; cooled to −50° C. at a rate of 40° C./min; then heated at a rate of 20° C./min up to a final temperature of 325° C. The T_{g} is reported from the inflection point of the transitions as the average value.

FT-IR:

IR spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. An attenuated total reflectance (ATR) accessory (Thunderdome™, Thermo Spectra-Tech), with a germanium (Ge) crystal was used for data acquisition. The spectra represent 256 co-added scans collected at a spectral resolution of 4 cm⁻¹. A background data set was acquired with a clean Ge crystal. Log 1/R(R=reflectance) spectra were acquired by taking a ratio of these two data sets against each other. Wavelength calibration was performed using polystyrene. Data were analyzed and peak lists were generated by using Omnic v. 7.2 software.

Thermogravimetric (TGA):

TGA Analyses were carried out on a TA Instruments 2950 thermogravimetric analyzer. The calibration standards were nickel and Alumel™. Each sample was placed in an aluminum sample pan and inserted into the TG furnace. Samples were first equilibrated at 25° C. or started directly from ambient conditions, then heated under a stream of nitrogen at a heating rate of 10° C./min, up to a final temperature of 325° C. unless specified otherwise.

Nuclear Magnetic Resonance (NMR):

The solution ¹H NMR spectra were acquired at ambient temperature on a Varian ^{UNITY}INOVA-400 spectrometer. Samples were prepared for NMR spectroscopy as ˜5-50 mg solutions in the appropriate deuterated solvent. The specific acquisition parameters are listed on the plot of the first full spectrum of each sample in the data section. Samples were prepared for solid-state NMR spectroscopy by packing them into 4 mm PENCIL type zirconia rotors. The specific acquisition parameters are listed on the plot of the first full spectrum of each sample in the data section.

### X-Ray Powder Diffraction (XRPD):

Inel XRG-3000: X-ray powder diffraction analyses were performed on an Inel XRG-3000 diffractometer, equipped with a curved position-sensitive detector with a 20 range of 120°. Real time data was collected using Cu Kα radiation at a resolution of 0.03 °2θ. The tube voltage and amperage were set to 40 kV and 30 mA, respectively. Patterns are displayed from 2.5 to 40 °2θ to facilitate direct pattern comparisons. Samples were prepared for analysis by packing them into thin-walled glass capillaries. Each capillary was mounted onto a goniometer head that is motorized to permit spinning of the capillary during data acquisition. Instrument calibration was performed daily using a silicon reference standard.

PANalytical X′Pert Pro:

XRPD patterns were collected using a PANalytical X′Pert Pro diffractometer. The specimen was analyzed using Cu radiation produced using an Optix long fine-focus source. An elliptically graded multilayer mirror was used to focus the Cu Kα X-rays of the source through the specimen and onto the detector. The specimen was sandwiched between 3-micron thick films, analyzed in transmission geometry, and rotated parallel to the diffraction vector to optimize orientation statistics. A beam-stop and helium purge was used to minimize the background generated by air scattering. Soller slits were used for the incident and diffracted beams to minimize axial divergence. Diffraction patterns were collected using a scanning position-sensitive detector (X'Celerator) located 240 mm from the specimen. The data-acquisition parameters of each diffraction pattern are displayed above the image of each pattern in appendix C. Prior to the analysis a silicon specimen (NIST standard reference material 640c) was analyzed to verify the position of the silicon 111 peak.

### X-ray Single Crystal Diffraction:

Data Collection:

Single crystal X-ray diffraction were performed by mounting a yellow needle of OSI-906 on a glass fiber in random orientation. Preliminary examination and data collection were performed with Mo K_{α} radiation (λ=0.71073 Å) on a Nonius KappaCCD diffractometer equipped with a graphite crystal, incident beam monochromator. Refinements were performed on an LINUX PC using SHELX97. (see Sheldrick, G. M. SHELX97, A Program for Crystal Structure Refinement, University of Gottingen, Germany, 1997) Cell constants and an orientation matrix for data collection were obtained from least-squares refinement using the setting angles of 16163 reflections in the range 2°<θ<27°. The refined mosaicity from Denzo/Scalepack is 0.69° indicating moderate crystal quality. (see Otwinowski, Z.; Minor, W. Methods Enzymol., 276, 307, 1997) The space group was determined by the program XPREP. (see Bruker, XPREP in SHELXTL v. 6.12., (see Bruker AXS Inc., Madison, Wis., USA, 2002) From the systematic presence of the following conditions: h0I h+I=2n; 0k0 k; =2n and from subsequent least-squares refinement, the space group was determined to be P2₁/n (SSCI Data Summary to OSI Pharmaceuticals, Standard Polymorph Screen of OSI-906, DS-5274.01, 2007). The data were collected to a maximum 2θ value of 55.03, at a temperature of 150±1 K.

Data Reduction:

Frames were integrated with DENZO-SMN. (see Otwinowski, Z.; Minor, W. Methods Enzymol., 276, 307, 1997) A total of 16163 reflections were collected, of which 4065 were unique. Lorentz and polarization corrections were applied to the data. The linear absorption coefficient is 0.078 mm⁻¹ for Mo K_{α} radiation. An empirical absorption correction using SCALEPACK (see Otwinowski, Z.; Minor, W. Methods Enzymol., 276, 307, 1997) was applied. Transmission coefficients ranged from 0.967 to 0.991. A secondary extinction correction was applied. (see Sheldrick, G. M. SHELX97, A Program for Crystal Structure Refinement, University of Gottingen, Germany, 1997) The final coefficient, refined in least-squares, was 0.0190 (in absolute units). Intensities of equivalent reflections were averaged. The agreement factor for the averaging was 7.7% based on intensity.

Structure Solution and Refinement:

The structure was solved by direct methods using known methods. (see Burla, M. C., Caliandro, R., Camalli, M., Carrozzini, B., Cascarano, G. L., De Caro, L., Giacovazzo, C., Polidori, G., and Spagna, R., J. Appl. Cryst., 38, 381, 2005) The remaining atoms were located in succeeding difference Fourier syntheses. Hydrogen atoms were included in the refinement but restrained to ride on the atom to which they are bonded. The structure was refined in full-matrix least-squares by minimizing the function:

Σw(|Fₒ|²−|Fₑ|²)²

The weight w is defined as 1/[σ²(Fₒ²)+(0.1528P)²+(0.000P)], where P=(Fₒ²+2F_{c}²)/3. Scattering factors were taken from the “International Tables for Crystallography.” (International Tables for Crystallography, Vol. C, Kluwer Academic Publishers: Dordrecht, The Netherlands, Tables 4.2.6.8 and 6.1.1.4, 1992). Of the 4065 reflections used in the refinements, only the reflections with Fₒ²>2σ(Fₒ²) were used in calculating R. A total of 3142 reflections were used in the calculation. The final cycle of refinement included 410 variable parameters and converged (largest parameter shift was essentially equal to its estimated standard deviation) with unweighted and weighted agreement factors of:

R=Σ|Fₒ−F_{c}|/ΣFₒ=0.070

R_{w}={square root over ((Σw(Fₒ²−F_{c}²)²/Σw(Fₒ²)²))}=0.182

The standard deviation of an observation of unit weight was 1.009. The highest peak in the final difference Fourier had a height of 0.28 e/Å³. The minimum negative peak had a height of −0.46 e/Å³.

ORTEP and Packing Diagrams: The ORTEP diagram was prepared using ORTEP III (Johnson, C. K. ORTEPIII, Report ORNL-6895, Oak Ridge National Laboratory, TN, U.S.A. 1996; OPTEP-3 for Windows V1.05, Farrugia, L. J., J. Appl. Cryst., 30, 565, 1997) program within the PLATON (Spek, A. L. PLUTON. Molecular Graphics Program. Univ. of Ultrecht, The Netherlands 1991; Spek, A. L. Acta Crystallogr., A46, C34, 1990) software package. Atoms are represented by 50% probability anisotropic thermal ellipsoids. Packing diagrams were prepared using CAMERON (Watkin, D. J.; Prout, C. K.; Pearce, L. J. CAMERON, Chemical Crystallography Laboratory, University of Oxford, Oxford, 1996) modeling software. Assessment of chiral centers, void calculations and additional figures were performed with the PLATON (Watkin, D. J., Prout, C. K., Pearce, L. J., CAMERON, Chemical Crystallography Laboratory, University of Oxford, Oxford, 1996) software package. Absolute configuration is evaluated using the specification of molecular chirality rules (Chan, R. S., Ingold, C., Prelog, V., Angew. Chem. Intern. Ed., Eng, 5, 385, 1966; Prelog, V. G. Helmchen, Angew. Chem. Intern. Ed. Eng., 21, 567, 1982). Additional figures were also generated with the Mercury 1.5 (Macrae, C. F. et. al., J. Appl. Cryst., 39, 453-457, 2006) visualization package. Hydrogen bonds are represented as dashed lines.

### Instrumental Techniques (Thermodynamic Stability Experiments—Forms A-F):

| Instrument | Vendor/Model # |
|---|---|
| Differential Scanning | Mettler 822^{e} DSC, Mettler DSC1 |
| Calorimeter | |
| Thermal Gravimetric | Mettler 851^{e} SDTA/TGA |
| Analyzer | |
| X-Ray Powder | PANalytical CubixPro |
| Diffractometer | |
| Nuclear Magnetic | 500 MHz Bruker AVANCE |
| Resonance Spectrometer | |
| Gravimetric Moisture | Hiden IGAsorp Moisture Sorption |
| Sorption | Instrument |
| FTIR Spectrometer | Thermo Nicolet Avatar 370 |
| Raman Spectrometer | Kaiser RXN1 |
| Optical Microscope | Leica DMRB Polarized Microscope |
| Karl Fischer | Mettler Toledo 756 |
| Laser Diffraction | Malvern Mastersizer S |

Differential Scanning Calorimetry Analysis:

Differential scanning calorimetry (DSC) analyses were carried out on the samples “as is”. Samples were weighed in an aluminum pan, covered with a pierced lid, and then crimped. Analysis conditions were 30-105, 30-300, 30-350° C. at 10° C./min. In addition, isothermal holds were performed for a duration of five minutes at 105° C. and 200° C.

Thermal Gravimetric Analysis:

Thermal gravimetric analysis (TGA) analyses were carried out on the samples “as is”. Samples were weighed in an alumina crucible and analyzed from 30° C.-230° C. and 30° C.-300° C. at 10° C./min.

X-Ray Powder Diffraction:

Samples were analyzed “as is”. Samples were placed on Si zero-return ultra-micro sample holders. Analysis was performed using a 10 mm irradiated width and the following parameters were set within the hardware/software:

X-ray tube: Cu KV, 45 kV, 40 mA

### Detector: X′Celerator

### ASS Primary Slit: Fixed 1°

Divergence Slit (Prog): Automatic—5 mm irradiated length
Soller Slits: 0.02 radian
Scatter Slit (PASS): Automatic—5 mm observed length

### Scan Range: 3.0-45.0°

### Scan Mode Continuous

### Step Size: 0.02°

### Time per Step: 10 s

### Active Length: 2.54°

Following analysis the data was converted from adjustable to fixed slits using the X'Pert HighScore Plus software with the following parameters:

### Fixed Divergence Slit Size: 1.00°, 1.59 mm

### Crossover Point: 44.3° Omega

Nuclear Magnetic Resonance:

Acquisition of ¹H NMR spectra was performed 2-10 mg of sample dissolved in 0.8 mL of DMSO-d₆. Spectra were acquired with 32 to 64 scans and a pulse delay of 1.0 s with a (30°) pulse width.

### Instrumental Techniques (Quantitative Determination of Forms A, C, and D in OSI-906 by Raman Spectroscopy):

Raman Spectroscopy: Acquisition of Raman Spectra was performed on a Kaiser Raman WorkStation equipped with PhAT probe, or equivalent.

Software: HoloGRAMS 4.1 or equivalent, GRAMS/AI 7.02 or equivalent TQ Analyst 7.1 or equivalent.
Raman Source: 785 nm laser.
Spectral Range: greater than 300-1800 cm-1.
Sample spot size: 1.2 m.

### Single Exposure Time: 0.1 sec.

### Accu ms: 24.

Enabled Exposure options: Cosmic Ray filtering, Dark Subtraction, and Intensity Calibration.

### Preparation and Characterization

In the following experimental examples Tables 1-20 disclose XRPD, IR and single crystal X-ray diffraction data obtained during characterization of Examples 1-8, respectively.

The following description briefly describes Tables 1-20.

Table 1: XRPD data for Form A.

Table 2: IR data for Form A.

Table 3: XRPD data for Form B.

Table 4: IR data for Form B.

Table 5: XRPD data for Form C.

Table 6: IR data for Form C.

Table 7: XRPD data for Form D.

Table 8: IR data for Form D.

Table 9: XRPD data for Form E.

Table 10: IR data for Form E.

Table 11: XRPD data for Form F.

Table 12: IR data for Form F.

Table 13: XRPD data for Form G.

Table 14: XRPD data for Form H.

Table 15: Crystal data and data collection Parameters for OSI-906 Form H.

Table 16: Positional parameters and their estimated standard deviations for OSI-906 Form H.

Table 17: Bond distances in angstroms for OSI-906 Form H.

Table 18: Bond angles in degrees for OSI-906 Form H.

Table 19: Hydrogen bond distances in angstroms and angles in degrees for OSI-906 Form H.

Table 20: Torsion angles in degrees for OSI-906 Form H.

In the following experimental examples Tables 21-26 disclose stability data including XRPD and ¹H-NMR, obtained during thermodynamic stability experiments of Forms A, B, C, D, E, and F, respectively. The following description briefly describes Tables 21-26.

Table 21: Solid State Stability of Form A and Solid State Stability of Forms C+D.

Table 22: Slurries of OSI-906 Solid Forms.

Table 23: Refluxing/Stability Experiments.

Table 24: Isolation of Form F (IPA Solvate).

Table 25: Additional Experiments to Isolate OSI-906 Solid Forms.

Table 26: Physical stability studies of OSI-906 solid forms.

In the following experimental examples Tables 27-30 disclose Raman spectra, obtained during the Quantitative Determination of Forms A, C, and D in OSI-906 by Raman

Spectroscopy. The following description briefly describes Tables 27-30.

Table 27: Summary of calibration sample preparation.

Table 28: Summary of validation sample preparation.

Table 29: Summary of Accuracy results with Form C.

Table 30: Summary of Accuracy results with Form D.

Generally, the process of preparing the polymorphs of OSI-906 (cis-3-[8-amino-1-(2-phenyl-quinolin-7-yl)-imidazo[1,5-a]pyrazin-3-yl]-1-methylcyclobutanol) includes:

Preparing a solution or slurry of OSI-906 in a solvent selected from suitable organic solvent such as but not limited to an alcohol, aqueous alcohol or polar solvent at a first predetermined temperature to form a solution; allowing solution to cool or maintain at ambient for a second predetermined temperature whereby a portion or all of OSI-906 crystallizes; and wherein said first predetermined temperature is between ambient and 120° C.; and said second predetermined temperature is between ambient and −20° C.

The present invention provides for methods of preparing OSI-906 Forms A-G as illustrated in Scheme 1.

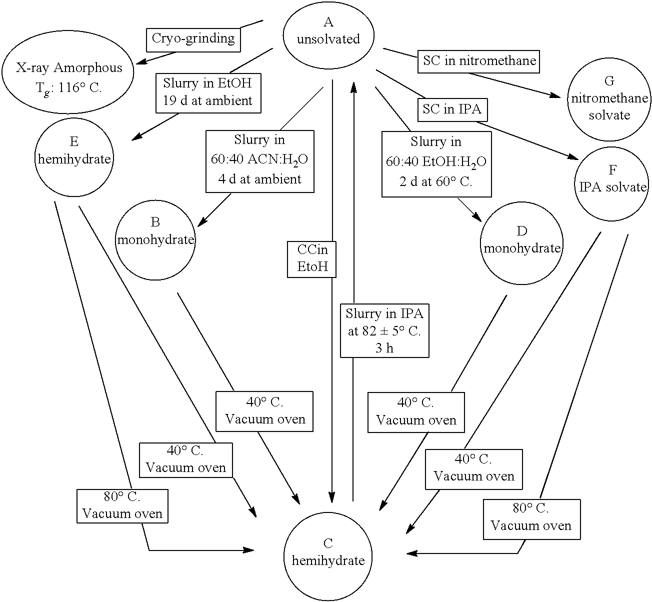

### Polymorph Screen

Both thermodynamic and kinetic crystallization techniques were employed. These techniques are described in more detail below. Once solid samples were harvested from crystallization attempts, they were either examined under a microscope for birefringence and morphology or observed with the naked eye. Any crystalline shape was noted, but sometimes the solid exhibited unknown morphology, due to small particle size. Solid samples were then analyzed by XRPD, and the crystalline patterns compared to each other to identify new crystalline forms.

Crash Cool (CC):

Saturated solutions were prepared in various solvents at elevated temperatures and filtered through a 0.2-μm nylon filter into a vial. Vials were then either placed in a (dry ice+isopropanol) cooling bath or placed in the freezer. The resulting solids were isolated by filtration and dried prior to analysis.

### Cryo-Grinding:

A solid sample was placed into a stainless steel grinding cup with a grinding rod. The sample was then ground on a SPEX Certiprep model 6750 Freezer Mill for a set amount of time. The ground solid was isolated and stored in freezer over desiccant until analyzed.

Fast Evaporation (FE):

Solutions were prepared in various solvents and sonicated between aliquot additions to assist in dissolution. Once a mixture reached complete dissolution, as judged by visual observation, the solution was filtered through a 0.2-μm nylon filter. The filtered solution was allowed to evaporate at ambient in an uncapped vial. The solids that formed were isolated and analyzed.

Freeze Drying:

1,4-dioxane solutions were prepared, filtered through a 0.2-μm nylon filter, and frozen in a vial immersed in a bath of liquid nitrogen or dry ice and isopropanol. The vial containing the frozen sample was attached to a Flexi-Dry lyophilizer and dried for a measured time period. After drying, the solids were isolated and stored in the freezer over desiccant until used.

Melt/Quench:

A portion of OSI-906 was dispensed in an even layer into a scintillation vial. The vial was capped and heated within an oil bath on a hot plate until the solids had completely melted. The vial was then removed from the hot plate and placed in the hood or a bath of liquid nitrogen to cool.

Slow Cool (SC):

Saturated solutions were prepared in various solvents at elevated temperatures and filtered through a 0.2-μm nylon filter into an open vial while still warm. The vial was covered and allowed to cool slowly to room temperature. The presence or absence of solids was noted. If there were no solids present, or if the amount of solids was judged too small for XRPD analysis, the vial was placed in a refrigerator. Again, the presence or absence of solids was noted and if there were none, the vial was placed in a freezer. Solids that formed were isolated by filtration and allowed to dry prior to analysis.

Slow Evaporation (SE):

Solutions were prepared in various solvents and sonicated between aliquot additions to assist in dissolution. Once a mixture reached complete dissolution, as judged by visual observation, the solution was filtered through a 0.2-μm nylon filter. The filtered solution was allowed to evaporate at ambient in a vial covered with aluminum foil perforated with pinholes. The solids that formed were isolated and analyzed.

Slurry Experiments:

Solutions were prepared by adding enough solids to a given solvent so that excess solids were present. The mixture was then agitated in a sealed vial at ambient temperature or an elevated temperature. After a given period of time, the solids were isolated by vacuum filtration.

The methods and materials of the invention are further detailed in the following nonlimiting examples.

### Example 1

### Preparation of OSI-906 Form A

a) OSI-906 was dissolved in water adjusted to pH of 3 and then added IPA. Then adjusted the solution to pH 5 to precipitate the product. The solid is isolated under filtration and dried under vacuum. Then the solid is suspended in IPA to give a slurry. The solid is isolated under filtration and dried under vacuum to afford Form A.

b) To a sealable 20 mL glass vial transferred 26.6 mg of OSI-906 which was dissolved in 7.0 mL EtOH to give a slurry, which was sonicated followed by addition of 256.9 mg of OSI-906. Solution was agitated in sealed vial at ambient. Solution was seeded with Form E. Then after 19 days the resultant solid was isolated by vacuum filtration to give 245.8 mg of Form A.

c) To a sealable 20 mL glass vial was added 71.8 mg of Form C, which was suspended in 0.87 mL of IPA and then stirred and heated solution for 3 h at 82° C. The solids were filtered under nitrogen, washed with 0.1 mL IPA and dried under vacuum at 40° C. for about 20 hours to give a light yellow solid as Form A.

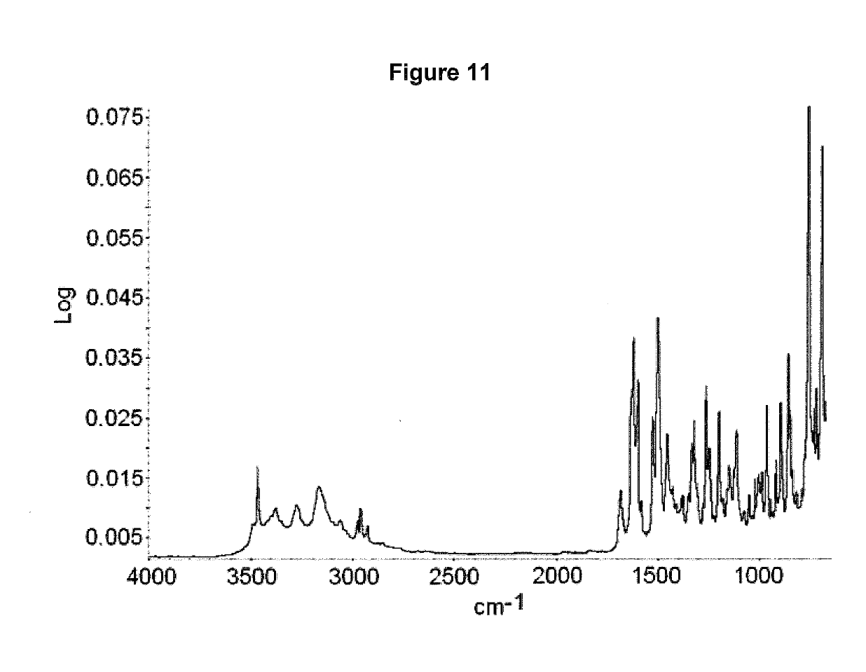

The XRPD, IR, DSC, TGA, and ¹H NMR (DMSO-d₆) of the sample are recorded and are reproduced in FIGS. 2, 3, 11, 17, 18, and 31 and Tables 1 and 2.

TABLE 1

| | | Intensity |
|---|---|---|
| °2θ | d space (Å) | (%) |
| 8.3 ± 0.1 | 10.687 ± 0.131 | 3 |
| 8.7 ± 0.1 | 10.164 ± 0.118 | 8 |
| 10.5 ± 0.1 | 8.442 ± 0.081 | 7 |
| 12.4 ± 0.1 | 7.114 ± 0.057 | 37 |
| 12.6 ± 0.1 | 7.029 ± 0.056 | 25 |
| 13.1 ± 0.1 | 6.761 ± 0.052 | 14 |
| 13.9 ± 0.1 | 6.369 ± 0.046 | 4 |
| 15.0 ± 0.1 | 5.923 ± 0.040 | 4 |
| 16.3 ± 0.1 | 5.443 ± 0.033 | 8 |
| 16.6 ± 0.1 | 5.339 ± 0.032 | 32 |
| 16.9 ± 0.1 | 5.232 ± 0.031 | 10 |
| 17.4 ± 0.1 | 5.085 ± 0.029 | 7 |
| 17.6 ± 0.1 | 5.026 ± 0.028 | 6 |
| 18.5 ± 0.1 | 4.800 ± 0.026 | 31 |
| 19.4 ± 0.1 | 4.583 ± 0.024 | 100 |
| 19.7 ± 0.1 | 4.513 ± 0.023 | 16 |
| 20.2 ± 0.1 | 4.399 ± 0.022 | 96 |
| 21.0 ± 0.1 | 4.236 ± 0.020 | 22 |
| 21.1 ± 0.1 | 4.206 ± 0.020 | 24 |
| 22.0 ± 0.1 | 4.046 ± 0.018 | 32 |
| 22.3 ± 0.1 | 3.995 ± 0.018 | 16 |
| 22.8 ± 0.1 | 3.897 ± 0.017 | 8 |
| 24.2 ± 0.1 | 3.682 ± 0.015 | 4 |
| 25.0 ± 0.1 | 3.556 ± 0.014 | 10 |
| 25.3 ± 0.1 | 3.515 ± 0.014 | 9 |
| 25.6 ± 0.1 | 3.485 ± 0.013 | 8 |
| 26.3 ± 0.1 | 3.386 ± 0.013 | 11 |
| 26.6 ± 0.1 | 3.354 ± 0.012 | 7 |
| 27.2 ± 0.1 | 3.276 ± 0.012 | 17 |
| 27.4 ± 0.1 | 3.254 ± 0.012 | 16 |
| 27.6 ± 0.1 | 3.232 ± 0.012 | 15 |
| 27.9 ± 0.1 | 3.201 ± 0.011 | 9 |
| 29.3 ± 0.1 | 3.050 ± 0.010 | 8 |
| 29.7 ± 0.1 | 3.012 ± 0.010 | 7 |

TABLE 2

| Position (cm⁻¹) | Intensity |
|---|---|
| 695.4 | 0.102 |
| 722.2 | 0.041 |
| 741.9 | 0.0565 |
| 763 | 0.0906 |
| 779.7 | 0.0244 |
| 815.7 | 0.0195 |
| 837.4 | 0.0162 |
| 854.2 | 0.0501 |
| 891.5 | 0.0538 |
| 902.3 | 0.0276 |
| 924.9 | 0.0128 |
| 941.2 | 0.0275 |
| 955 | 0.023 |
| 974.2 | 0.0074 |
| 1002.8 | 0.0317 |
| 1023.7 | 0.0119 |
| 1055 | 0.0114 |
| 1077.7 | 0.007 |
| 1113.3 | 0.0208 |
| 1148.5 | 0.0465 |
| 1189.3 | 0.0088 |
| 1248.9 | 0.0496 |
| 1282.1 | 0.013 |
| 1302.1 | 0.0085 |
| 1317.4 | 0.0243 |
| 1331.4 | 0.0271 |
| 1382.2 | 0.0213 |
| 1403.6 | 0.019 |
| 1427.3 | 0.0386 |
| 1442.4 | 0.0327 |
| 1449.4 | 0.0345 |
| 1460.2 | 0.026 |
| 1489.4 | 0.0747 |
| 1526.9 | 0.018 |
| 1581.6 | 0.0122 |
| 1600.9 | 0.0493 |
| 1613.8 | 0.067 |
| 1829.1 | 0.0038 |
| 2564.6 | 0.0038 |
| 2668.6 | 0.004 |
| 2825.7 | 0.0073 |
| 2938.3 | 0.0121 |
| 2966.4 | 0.0121 |
| 3108.2 | 0.0122 |
| 3365.3 | 0.0126 |
| 3486.8 | 0.0206 |

### Example 2

### Preparation of OSI-906 Form B

To a sealable 20 mL glass vial was added 23.7 mg of OSI-906 and 8 mL of 60:40 (v/v) acetonitrile:water to form a solution after sonication. Then added 248.4 mg OSI-906 and agitated slurry in sealed vial. Then solution was seeded with Form B. Then after 4 days the resultant solid was isolated by filtration to give 257.2 mg of Form B.

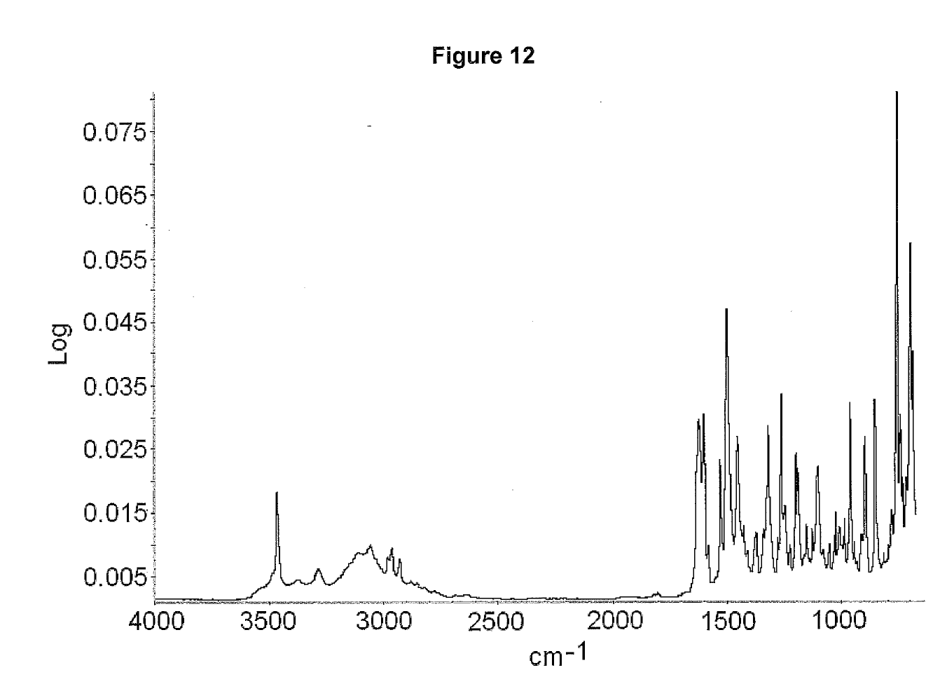

The XRPD, IR, DSC, TGA, and ¹H NMR (DMSO-d₆) of the sample are recorded and are reproduced in FIGS. 2, 4, 12, 19, 20, and 32 and Tables 3 and 4.

TABLE 3

| | | Intensity |
|---|---|---|
| °2θ | d space (Å) | (%) |
| 8.4 ± 0.1 | 10.477 ± 0.125 | 13.08 |
| 8.9 ± 0.1 | 9.981 ± 0.114 | 34.17 |
| 10.1 ± 0.1 | 8.767 ± 0.088 | 70.98 |
| 10.6 ± 0.1 | 8.346 ± 0.079 | 46.59 |
| 11.2 ± 0.1 | 7.900 ± 0.071 | 100 |
| 13.3 ± 0.1 | 6.642 ± 0.050 | 67.8 |
| 13.8 ± 0.1 | 6.399 ± 0.046 | 38.24 |
| 15.3 ± 0.1 | 5.776 ± 0.038 | 45.71 |
| 16.0 ± 0.1 | 5.539 ± 0.035 | 34.28 |
| 16.3 ± 0.1 | 5.428 ± 0.033 | 64.43 |
| 17.2 ± 0.1 | 5.147 ± 0.030 | 8.2 |
| 17.7 ± 0.1 | 5.000 ± 0.028 | 43.83 |
| 18.0 ± 0.1 | 4.942 ± 0.027 | 35.46 |
| 18.5 ± 0.1 | 4.799 ± 0.026 | 24.29 |
| 19.3 ± 0.1 | 4.599 ± 0.024 | 9.16 |
| 20.1 ± 0.1 | 4.409 ± 0.022 | 29.72 |
| 20.4 ± 0.1 | 4.351 ± 0.021 | 31.03 |
| 21.2 ± 0.1 | 4.187 ± 0.020 | 37.48 |
| 21.5 ± 0.1 | 4.129 ± 0.019 | 43.23 |
| 21.8 ± 0.1 | 4.068 ± 0.018 | 45.02 |
| 22.3 ± 0.1 | 3.992 ± 0.018 | 67.43 |
| 22.4 ± 0.1 | 3.960 ± 0.017 | 63.64 |
| 23.6 ± 0.1 | 3.776 ± 0.016 | 15.57 |
| 24.0 ± 0.1 | 3.716 ± 0.015 | 27.67 |
| 24.4 ± 0.1 | 3.653 ± 0.015 | 59.26 |
| 24.8 ± 0.1 | 3.583 ± 0.014 | 6.84 |
| 25.5 ± 0.1 | 3.496 ± 0.014 | 38.82 |
| 26.0 ± 0.1 | 3.428 ± 0.013 | 27.59 |
| 26.2 ± 0.1 | 3.405 ± 0.013 | 26.93 |
| 26.7 ± 0.1 | 3.338 ± 0.012 | 19.34 |
| 27.1 ± 0.1 | 3.294 ± 0.012 | 19.25 |
| 27.4 ± 0.1 | 3.259 ± 0.012 | 17.41 |
| 27.8 ± 0.1 | 3.210 ± 0.011 | 57.42 |
| 29.1 ± 0.1 | 3.071 ± 0.010 | 32.78 |
| 29.6 ± 0.1 | 3.019 ± 0.010 | 9.01 |
| 30.0 ± 0.1 | 2.984 ± 0.010 | 5.85 |

TABLE 4

| Position (cm⁻¹) | Intensity |
|---|---|
| 694.5 | 0.07 |
| 720.9 | 0.0297 |
| 733.3 | 0.0264 |
| 759.8 | 0.0767 |
| 780.9 | 0.0187 |
| 794.3 | 0.013 |
| 819.7 | 0.0124 |
| 839.4 | 0.0162 |
| 852.3 | 0.026 |
| 861.9 | 0.0355 |
| 896.3 | 0.0275 |
| 921.8 | 0.0177 |
| 942.9 | 0.0112 |
| 966.1 | 0.0267 |
| 988.9 | 0.0158 |
| 1006.5 | 0.0154 |
| 1025.6 | 0.0147 |
| 1037.6 | 0.0084 |
| 1053.3 | 0.0119 |
| 1077.6 | 0.0092 |
| 1116.6 | 0.0227 |
| 1128.4 | 0.0157 |
| 1151.1 | 0.0168 |
| 1160.1 | 0.013 |
| 1182 | 0.0116 |
| 1200.2 | 0.0261 |
| 1226.9 | 0.0086 |
| 1248.9 | 0.02 |
| 1265.4 | 0.0303 |
| 1280.1 | 0.0105 |
| 1320.9 | 0.0244 |
| 1330.8 | 0.0207 |
| 1349.6 | 0.0122 |
| 1376.7 | 0.0119 |
| 1386.5 | 0.0118 |
| 1411.7 | 0.0103 |
| 1428.3 | 0.0134 |
| 1454 | 0.0221 |
| 1499.3 | 0.0419 |
| 1525 | 0.0251 |
| 1583.4 | 0.0108 |
| 1599.9 | 0.0315 |
| 1620.5 | 0.0382 |
| 1683.3 | 0.0127 |
| 2851.8 | 0.0042 |
| 2927.7 | 0.0068 |
| 2961.7 | 0.0098 |
| 2976.2 | 0.0076 |
| 3062.6 | 0.0078 |
| 3166.1 | 0.0132 |
| 3276.9 | 0.0102 |
| 3378.4 | 0.0097 |
| 3469.1 | 0.0167 |
| — | — |

### Example 3

### Preparation of OSI-906 Form C

To a sealable 20 mL glass vial was added 24.3 mg of OSI-906 and 3.5 mL of EtOH. Agitated mixture at about 70° C. to form a solution. Then filtered solution through a pre-heated 0.2 μm nylon filter into a pre-cooled 20 mL glass vial within a cooling bath (dry ice+IPA) followed by cooling of filtrate to 0° C. The resultant solids were isolated by vacuum filtration to give Form C.

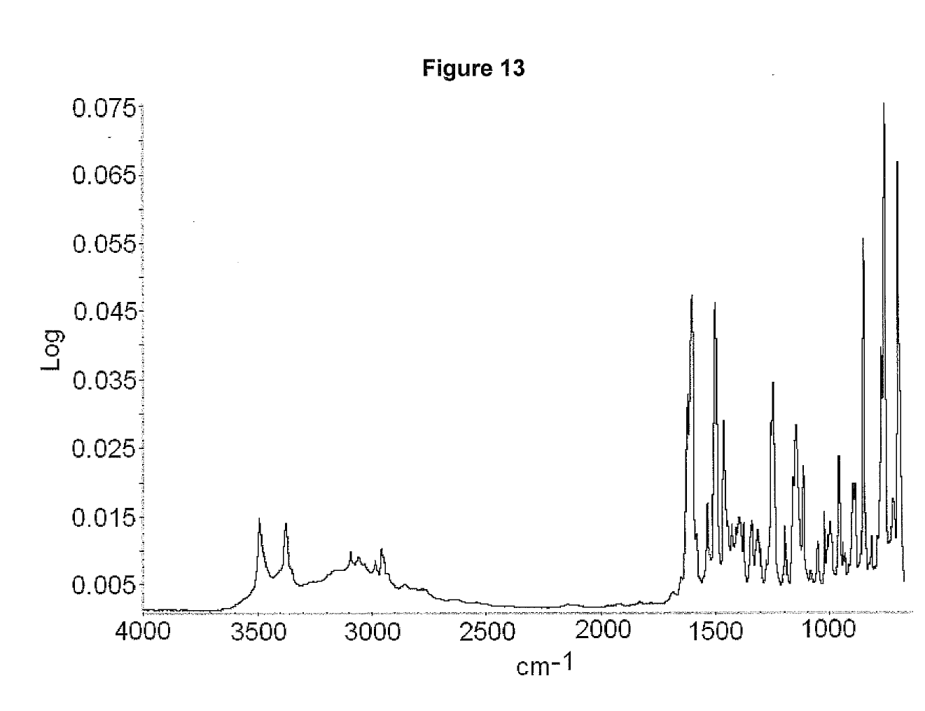

The XRPD, IR, DSC, TGA, and ¹H NMR (DMSO-d₆) of the sample are recorded and are reproduced in FIGS. 2, 5, 13, 21, 22, and 33 and Tables 5 and 6.

TABLE 5

| | | Intensity |
|---|---|---|
| °2θ | d space (Å) | (%) |
| 8.4 ± 0.1 | 10.517 ± 0.126 | 10 |
| 10.0 ± 0.1 | 8.828 ± 0.089 | 24 |
| 10.6 ± 0.1 | 8.375 ± 0.080 | 66 |
| 11.2 ± 0.1 | 7.919 ± 0.071 | 52 |
| 13.3 ± 0.1 | 6.660 ± 0.050 | 100 |
| 13.9 ± 0.1 | 6.357 ± 0.046 | 5 |
| 15.3 ± 0.1 | 5.785 ± 0.038 | 32 |
| 16.0 ± 0.1 | 5.550 ± 0.035 | 25 |
| 16.3 ± 0.1 | 5.426 ± 0.033 | 29 |
| 17.2 ± 0.1 | 5.144 ± 0.030 | 7 |
| 18.5 ± 0.1 | 4.804 ± 0.026 | 10 |
| 19.3 ± 0.1 | 4.590 ± 0.024 | 9 |
| 20.4 ± 0.1 | 4.343 ± 0.021 | 13 |
| 21.2 ± 0.1 | 4.186 ± 0.020 | 42 |
| 21.9 ± 0.1 | 4.055 ± 0.018 | 6 |
| 22.5 ± 0.1 | 3.953 ± 0.017 | 16 |
| 23.6 ± 0.1 | 3.774 ± 0.016 | 9 |
| 23.9 ± 0.1 | 3.717 ± 0.015 | 11 |
| 24.3 ± 0.1 | 3.657 ± 0.015 | 31 |
| 25.5 ± 0.1 | 3.495 ± 0.014 | 29 |
| 26.0 ± 0.1 | 3.422 ± 0.013 | 8 |
| 26.8 ± 0.1 | 3.326 ± 0.012 | 6 |
| 27.8 ± 0.1 | 3.208 ± 0.011 | 16 |
| 29.1 ± 0.1 | 3.071 ± 0.010 | 11 |
| 29.6 ± 0.1 | 3.020 ± 0.010 | 6 |

TABLE 6

| Position (cm⁻¹) | Intensity |
|---|---|
| 687.1 | 0.0405 |
| 699 | 0.0587 |
| 717.6 | 0.0201 |
| 730.8 | 0.0202 |
| 742.7 | 0.03 |
| 756.6 | 0.0825 |
| 780.8 | 0.015 |
| 791.9 | 0.0096 |
| 813.7 | 0.0084 |
| 853.5 | 0.0332 |
| 895.2 | 0.0268 |
| 911.7 | 0.0112 |
| 932.3 | 0.0071 |
| 943.2 | 0.01 |
| 960.6 | 0.032 |
| 985.8 | 0.0137 |
| 1009.8 | 0.0125 |
| 1026.4 | 0.0148 |
| 1053.3 | 0.0099 |
| 1080.5 | 0.0088 |
| 1104 | 0.022 |
| 1127.9 | 0.0121 |
| 1150.7 | 0.013 |
| 1189.4 | 0.0217 |
| 1200.3 | 0.0244 |
| 1226.2 | 0.0096 |
| 1248.8 | 0.0159 |
| 1263.5 | 0.0335 |
| 1278.2 | 0.0108 |
| 1319 | 0.0283 |
| 1341.5 | 0.012 |
| 1372.4 | 0.0116 |
| 1412.7 | 0.0087 |
| 1427.6 | 0.0126 |
| 1456 | 0.0267 |
| 1499.7 | 0.0471 |
| 1528.9 | 0.0231 |
| 1584.4 | 0.0096 |
| 1602.6 | 0.0303 |
| 1618.1 | 0.0284 |
| 1624.3 | 0.0294 |
| 2853.5 | 0.0036 |
| 2880.6 | 0.004 |
| 2930.1 | 0.0076 |
| 2963 | 0.0093 |
| 2981.7 | 0.0078 |
| 3059.3 | 0.0096 |
| 3109.3 | 0.0085 |
| 3284.3 | 0.0059 |
| 3374.6 | 0.0042 |
| 3465.2 | 0.018 |
| — | — |

### Example 4

### Preparation of OSI-906 Form D

To a sealable 20 mL glass vial was added 50.6 mg of OSI-906 and 5 mL of 60:40 (v/v) EtOH:water to give a slurry which was heated to about 60° C. Then added 261.2 mg of OSI-906 to solution and then agitated in the sealed vial and heated to about 60° C. Then seeded the solution with Form D and after 2 days the resultant solid was isolated by vacuum filtration to give 265.3 mg of Form D.

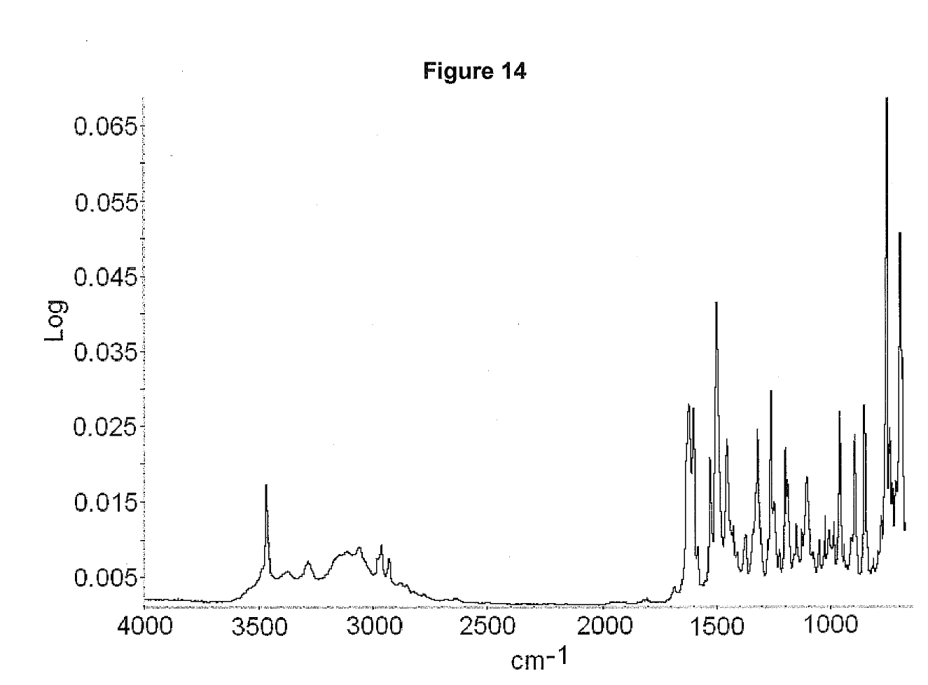

The XRPD, IR, DSC, TGA, and ¹H NMR (DMSO-d₆) of the sample are recorded and are reproduced in FIGS. 2, 6, 14, 23, 24, and 34 and Tables 7 and 8.

TABLE 7

| | | Intensity |
|---|---|---|
| °2θ | d space (Å) | (%) |
| 8.9 ± 0.1 | 9.981 ± 0.114 | 93.92 |
| 10.1 ± 0.1 | 8.793 ± 0.088 | 8.8 |
| 10.9 ± 0.1 | 8.139 ± 0.075 | 67.98 |
| 11.1 ± 0.1 | 7.964 ± 0.072 | 63.05 |
| 13.3 ± 0.1 | 6.672 ± 0.050 | 7.47 |
| 13.8 ± 0.1 | 6.399 ± 0.046 | 65.53 |
| 14.1 ± 0.1 | 6.277 ± 0.045 | 14.48 |
| 16.5 ± 0.1 | 5.379 ± 0.033 | 15.82 |
| 17.7 ± 0.1 | 5.000 ± 0.028 | 86.25 |
| 18.0 ± 0.1 | 4.942 ± 0.027 | 20.44 |
| 20.0 ± 0.1 | 4.442 ± 0.022 | 50.92 |
| 21.5 ± 0.1 | 4.141 ± 0.019 | 38.14 |
| 21.8 ± 0.1 | 4.073 ± 0.019 | 100 |
| 22.2 ± 0.1 | 4.003 ± 0.018 | 72.23 |
| 22.4 ± 0.1 | 3.966 ± 0.018 | 45.36 |
| 23.8 ± 0.1 | 3.734 ± 0.016 | 6.2 |
| 24.7 ± 0.1 | 3.600 ± 0.014 | 14.18 |
| 25.9 ± 0.1 | 3.436 ± 0.013 | 46.62 |
| 26.2 ± 0.1 | 3.405 ± 0.013 | 62.48 |
| 26.6 ± 0.1 | 3.345 ± 0.012 | 24.8 |
| 27.0 ± 0.1 | 3.298 ± 0.012 | 30.53 |
| 27.4 ± 0.1 | 3.259 ± 0.012 | 21.82 |
| 28.3 ± 0.1 | 3.150 ± 0.011 | 20.01 |
| 28.9 ± 0.1 | 3.086 ± 0.010 | 5.83 |
| 29.6 ± 0.1 | 3.016 ± 0.010 | 11.69 |
| 30.0 ± 0.1 | 2.984 ± 0.010 | 13.66 |

TABLE 8

| Position (cm⁻¹) | Intensity |
|---|---|
| 701.6 | 0.0672 |
| 724.3 | 0.0175 |
| 761.4 | 0.0756 |
| 773.1 | 0.0395 |
| 790.3 | 0.0118 |
| 803.5 | 0.0073 |
| 818.1 | 0.012 |
| 827.5 | 0.0078 |
| 852 | 0.0558 |
| 889.7 | 0.0196 |
| 896.8 | 0.0195 |
| 916.6 | 0.0077 |
| 933.8 | 0.0092 |
| 942.5 | 0.0109 |
| 958 | 0.0236 |
| 997.6 | 0.014 |
| 1008.4 | 0.012 |
| 1023.4 | 0.0155 |
| 1054.6 | 0.011 |
| 1083.2 | 0.0067 |
| 1116.5 | 0.0221 |
| 1145.9 | 0.0279 |
| 1160 | 0.0208 |
| 1194.6 | 0.0135 |
| 1248.2 | 0.0344 |
| 1256 | 0.028 |
| 1278.1 | 0.0083 |
| 1303 | 0.0099 |
| 1313 | 0.0128 |
| 1322.1 | 0.0111 |
| 1338.3 | 0.0141 |
| 1344.9 | 0.0135 |
| 1375.9 | 0.014 |
| 1395.9 | 0.0147 |
| 1410.9 | 0.0131 |
| 1427.2 | 0.0135 |
| 1444.8 | 0.0141 |
| 1461.9 | 0.0286 |
| 1498.8 | 0.0462 |
| 1534 | 0.0169 |
| 1583.4 | 0.0122 |
| 1601.3 | 0.0476 |
| 1620 | 0.0326 |
| 1648.1 | 0.0058 |
| 1683.1 | 0.0037 |
| 2856.7 | 0.0046 |
| 2958.8 | 0.0101 |
| 2987.3 | 0.0083 |
| 3059.3 | 0.0087 |
| 3094.4 | 0.0095 |
| 3377.4 | 0.0137 |
| 3493.9 | 0.0145 |

### Example 5

### Preparations of OSI-906 Form E

To a sealable 20 mL glass vial was added 21.4 mg of OSI-906 and 7 mL of EtOH to form a solution after sonication. Then added 6.0 mg of OSI-906 to give turbid solution. Then added 31.9 mg of OSI-906. Agitated slurry in sealed vial at ambient. After 19 days the resultant solid was isolated by vacuum filtration to give Form E.

To a 50 mL flask was added 265.1 mg OSI-906 and 40 mL EtOH to form a solution after agitation at 70° C. Filtered solution through pre-heated nylon filter into a pre-cooled 20 mL glass vial within a cooling bath (dry ice+IPA). Then solution was cooled in the freezer. Seeded solution with Form C. The resultant solid was isolated by vacuum filtration to give 257.0 mg of Form E.

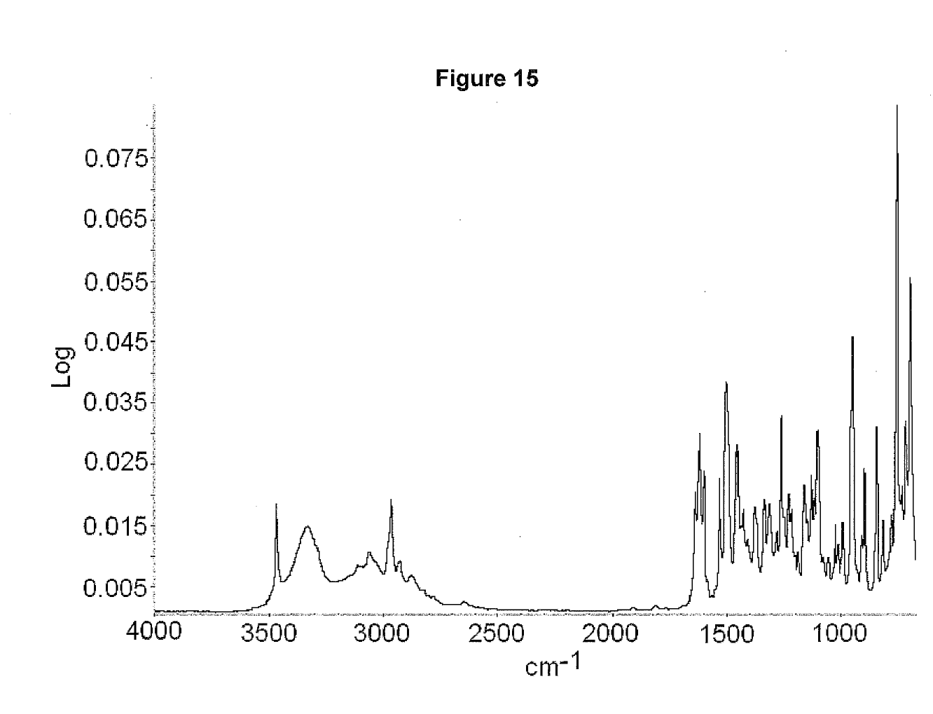

The XRPD, IR, DSC, TGA, and ¹H NMR (DMSO-d₆) of the sample are recorded and are reproduced in FIGS. 2, 7, 15, 25, 26, and 35 and Tables 9 and 10.

TABLE 9

| °2θ | d space (Å) | Intensity (%) |
|---|---|---|
| 6.3 ± 0.1 | 14.008 ± 0.225 | 100 |
| 6.8 ± 0.1 | 13.076 ± 0.196 | 17.91 |
| 8.3 ± 0.1 | 10.627 ± 0.129 | 25.99 |
| 10.1 ± 0.1 | 8.741 ± 0.087 | 3.15 |
| 10.6 ± 0.1 | 8.346 ± 0.079 | 2.01 |
| 11.2 ± 0.1 | 7.879 ± 0.071 | 2.17 |
| 11.8 ± 0.1 | 7.500 ± 0.064 | 1.92 |
| 12.8 ± 0.1 | 6.938 ± 0.055 | 35.24 |
| 13.2 ± 0.1 | 6.702 ± 0.051 | 16.26 |
| 13.5 ± 0.1 | 6.540 ± 0.048 | 82.37 |
| 14.5 ± 0.1 | 6.096 ± 0.042 | 8.88 |
| 15.4 ± 0.1 | 5.754 ± 0.037 | 4.5 |
| 16.0 ± 0.1 | 5.529 ± 0.034 | 1.96 |
| 16.3 ± 0.1 | 5.428 ± 0.033 | 3.42 |
| 16.6 ± 0.1 | 5.331 ± 0.032 | 4.14 |
| 17.0 ± 0.1 | 5.219 ± 0.031 | 4.43 |
| 17.4 ± 0.1 | 5.111 ± 0.029 | 22.23 |
| 18.0 ± 0.1 | 4.942 ± 0.027 | 14.36 |
| 18.2 ± 0.1 | 4.861 ± 0.027 | 12.18 |
| 18.9 ± 0.1 | 4.686 ± 0.025 | 23.18 |
| 19.4 ± 0.1 | 4.585 ± 0.024 | 3.69 |
| 20.1 ± 0.1 | 4.409 ± 0.022 | 18.31 |
| 20.6 ± 0.1 | 4.301 ± 0.021 | 15.02 |
| 21.2 ± 0.1 | 4.181 ± 0.020 | 46.65 |
| 21.8 ± 0.1 | 4.084 ± 0.019 | 7.21 |
| 22.3 ± 0.1 | 3.992 ± 0.018 | 2.58 |
| 23.2 ± 0.1 | 3.829 ± 0.016 | 4.65 |
| 23.8 ± 0.1 | 3.739 ± 0.016 | 19.95 |
| 24.1 ± 0.1 | 3.688 ± 0.015 | 7.27 |
| 24.5 ± 0.1 | 3.631 ± 0.015 | 11.54 |
| 25.1 ± 0.1 | 3.554 ± 0.014 | 6.76 |
| 25.6 ± 0.1 | 3.476 ± 0.013 | 7.43 |
| 26.3 ± 0.1 | 3.390 ± 0.013 | 9.77 |
| 26.9 ± 0.1 | 3.319 ± 0.012 | 16.28 |
| 27.2 ± 0.1 | 3.273 ± 0.012 | 13.4 |
| 28.0 ± 0.1 | 3.187 ± 0.011 | 4.65 |
| 28.7 ± 0.1 | 3.108 ± 0.011 | 5.63 |
| 29.4 ± 0.1 | 3.040 ± 0.010 | 4.4 |

TABLE 10

| Position (cm⁻¹) | Intensity |
|---|---|
| 687.8 | 0.0343 |
| 698.5 | 0.0508 |
| 718.6 | 0.0178 |
| 730.8 | 0.0173 |
| 742.7 | 0.0247 |
| 756.7 | 0.0701 |
| 780.9 | 0.0129 |
| 792.3 | 0.0082 |
| 814.3 | 0.0072 |
| 853.6 | 0.0281 |
| 895.3 | 0.0239 |
| 911.6 | 0.01 |
| 932.3 | 0.0064 |
| 943.1 | 0.0092 |
| 960.7 | 0.0268 |
| 985.9 | 0.0121 |
| 1009.1 | 0.0111 |
| 1026.3 | 0.013 |
| 1053.1 | 0.0097 |
| 1080.4 | 0.0082 |
| 1104.4 | 0.0182 |
| 1128 | 0.0112 |
| 1150.9 | 0.012 |
| 1189.5 | 0.0178 |
| 1200.3 | 0.0223 |
| 1226.4 | 0.0084 |
| 1249 | 0.0148 |
| 1263.7 | 0.0297 |
| 1278.2 | 0.0097 |
| 1319.3 | 0.0245 |
| 1341.3 | 0.0112 |
| 1372.8 | 0.0105 |
| 1412.6 | 0.0081 |
| 1427.6 | 0.0117 |
| 1455.9 | 0.023 |
| 1499.8 | 0.0416 |
| 1528.5 | 0.0209 |
| 1584.2 | 0.009 |
| 1602.3 | 0.0272 |
| 1623.4 | 0.0278 |
| 1683.9 | 0.0035 |
| 2853.5 | 0.0038 |
| 2880.2 | 0.0041 |
| 2930 | 0.0073 |
| 2962.8 | 0.0092 |
| 3059.6 | 0.0089 |
| 3111.3 | 0.0083 |
| 3283.4 | 0.0069 |
| 3374.9 | 0.0056 |
| 3465.6 | 0.0171 |

### Example 6

### Preparation of OSI-906 Form F

To a glass flask was added 267.0 mg of OSI-906 and 70 mL IPA to form a solution. Agitated solution and heated to 70° C. to give a turbid solution. Filtered solution through pre-heated nylon filter into a pre-heated 125 mL flask. Cooled slowly to ambient and seeded solution with Form F. Cooled solution in refrigerator and then in freezer. The resultant solids were isolated by vacuum filtration to give 207.9 mg of Form F.

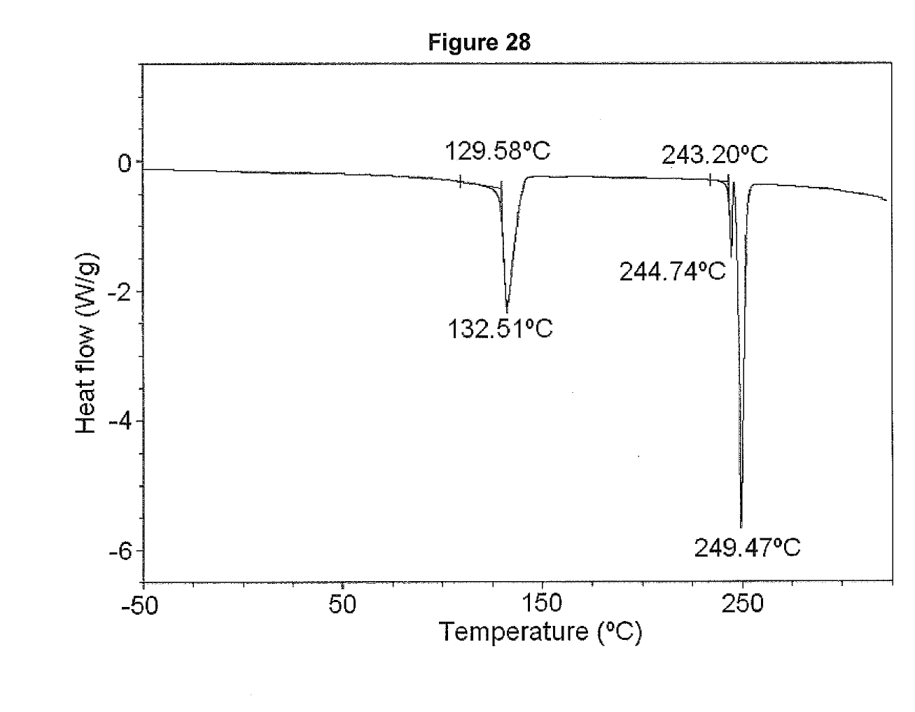

The XRPD, IR, DSC, TGA, and ¹H NMR (DMSO-d₆) of the sample are recorded and are reproduced in FIGS. 2, 8, 16, 27, 28, and 36 and Tables 11 and 12.

TABLE 11

| °2θ | d space (Å) | Intensity (%) |
|---|---|---|
| 6.0 ± 0.1 | 14.633 ± 0.246 | 100 |
| 6.6 ± 0.1 | 13.433 ± 0.207 | 7.92 |
| 8.9 ± 0.1 | 9.981 ± 0.114 | 19.19 |
| 11.8 ± 0.1 | 7.519 ± 0.064 | 35.93 |
| 13.3 ± 0.1 | 6.672 ± 0.050 | 66.66 |
| 14.4 ± 0.1 | 6.172 ± 0.043 | 16.29 |
| 14.7 ± 0.1 | 6.010 ± 0.041 | 23.68 |
| 16.2 ± 0.1 | 5.488 ± 0.034 | 7.66 |
| 17.7 ± 0.1 | 5.008 ± 0.028 | 68.53 |
| 18.2 ± 0.1 | 4.877 ± 0.027 | 6.26 |
| 18.6 ± 0.1 | 4.776 ± 0.026 | 3.99 |
| 19.2 ± 0.1 | 4.620 ± 0.024 | 16.79 |
| 19.7 ± 0.1 | 4.516 ± 0.023 | 50.54 |
| 20.3 ± 0.1 | 4.377 ± 0.021 | 7.41 |
| 20.7 ± 0.1 | 4.295 ± 0.021 | 4.5 |
| 23.2 ± 0.1 | 3.839 ± 0.016 | 9.84 |
| 23.8 ± 0.1 | 3.734 ± 0.016 | 39.97 |
| 24.6 ± 0.1 | 3.622 ± 0.015 | 17.84 |
| 25.6 ± 0.1 | 3.476 ± 0.013 | 17.59 |
| 26.5 ± 0.1 | 3.364 ± 0.013 | 27.62 |
| 27.1 ± 0.1 | 3.290 ± 0.012 | 16.22 |
| 27.6 ± 0.1 | 3.227 ± 0.011 | 4.13 |
| 29.0 ± 0.1 | 3.074 ± 0.010 | 5.15 |

TABLE 12

| Position (cm⁻¹) | Intensity |
|---|---|
| 699.3 | 0.0566 |
| 719.7 | 0.032 |
| 734.4 | 0.0212 |
| 757.9 | 0.0836 |
| 780.8 | 0.0164 |
| 816.8 | 0.0159 |
| 845.5 | 0.0315 |
| 898 | 0.0244 |
| 909.9 | 0.0132 |
| 950.6 | 0.0455 |
| 992.9 | 0.0153 |
| 1012.7 | 0.0116 |
| 1026.6 | 0.015 |
| 1057.2 | 0.0096 |
| 1079.5 | 0.0095 |
| 1102 | 0.0307 |
| 1117 | 0.0193 |
| 1128.9 | 0.0233 |
| 1147.4 | 0.0157 |
| 1160.9 | 0.0213 |
| 1187.5 | 0.0104 |
| 1219.1 | 0.0174 |
| 1229 | 0.02 |
| 1262 | 0.0332 |
| 1281.1 | 0.014 |
| 1312.4 | 0.0184 |
| 1334.5 | 0.0191 |
| 1377.3 | 0.0179 |
| 1409.6 | 0.0126 |
| 1428.7 | 0.0175 |
| 1456.2 | 0.028 |
| 1501.9 | 0.0382 |
| 1531.2 | 0.0225 |
| 1585 | 0.0059 |
| 1600.3 | 0.0238 |
| 1617.9 | 0.0298 |
| 1638 | 0.0204 |
| 2881 | 0.0067 |
| 2929.7 | 0.009 |
| 2966.6 | 0.019 |
| 3062.6 | 0.0105 |
| 3110.9 | 0.0083 |
| 3332.9 | 0.0147 |
| 3468.7 | 0.0186 |

### Example 7

### Preparation of OSI-906 Form G

To a glass flask was added 128.3 mg of OSI-906 and 75 mL nitromethane. Agitated solution and heated to 70° C. to give a turbid solution. Filtered turbid solution through a pre-heated nylon filter into a pre-heated 125 mL flask. Cooled solution to ambient and seeded with Form G. Cooled the solution in refrigerator and then in freezer. The resultant solids were isolated by vacuum filtration to give 67.6 mg of Form G.

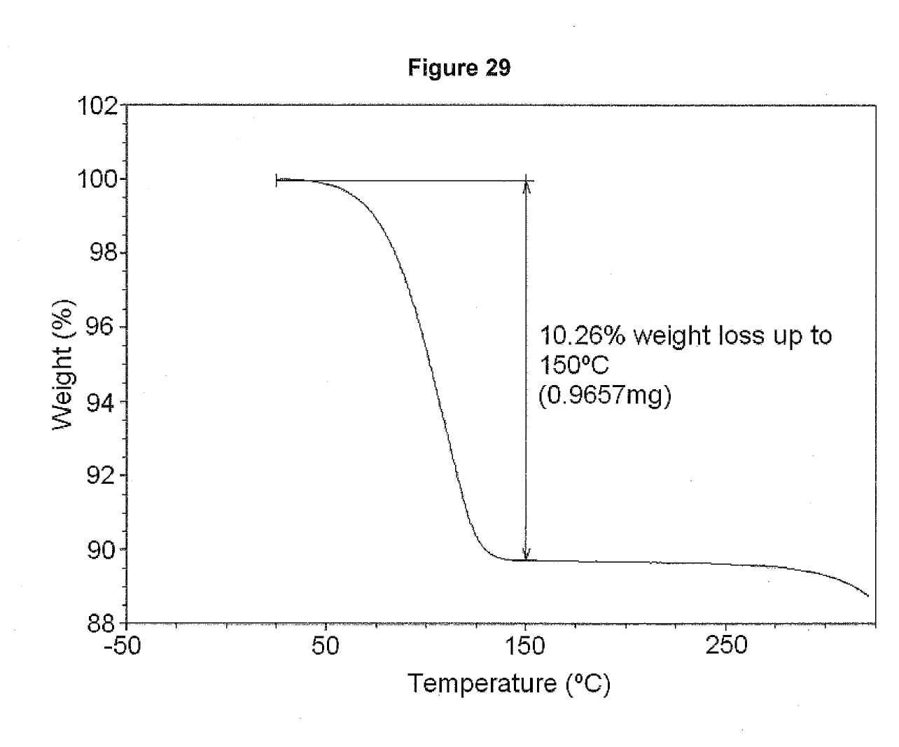

The XRPD, DSC, TGA, and ¹H NMR (DMSO-d₆) of the sample are recorded and are reproduced in FIGS. 2, 9, 29, 30, and 37 and Table 13.

TABLE 13

| °2θ | d space (Å) | Intensity (%) |
|---|---|---|
| 9.4 ± 0.1 | 9.409 ± 0.101 | 48.57 |
| 11.5 ± 0.1 | 7.695 ± 0.067 | 15.63 |
| 13.7 ± 0.1 | 6.468 ± 0.047 | 69.96 |
| 14.6 ± 0.1 | 6.059 ± 0.042 | 10.91 |
| 15.4 ± 0.1 | 5.743 ± 0.037 | 15.02 |
| 15.8 ± 0.1 | 5.591 ± 0.035 | 16.45 |
| 16.3 ± 0.1 | 5.428 ± 0.033 | 100 |
| 16.6 ± 0.1 | 5.341 ± 0.032 | 29.76 |
| 17.0 ± 0.1 | 5.210 ± 0.031 | 51.26 |
| 17.6 ± 0.1 | 5.042 ± 0.029 | 41.19 |
| 18.8 ± 0.1 | 4.723 ± 0.025 | 88.51 |
| 19.2 ± 0.1 | 4.613 ± 0.024 | 23.7 |
| 20.1 ± 0.1 | 4.409 ± 0.022 | 17.51 |
| 20.5 ± 0.1 | 4.332 ± 0.021 | 67.19 |
| 21.4 ± 0.1 | 4.152 ± 0.019 | 89.15 |
| 23.0 ± 0.1 | 3.859 ± 0.017 | 89.48 |
| 23.4 ± 0.1 | 3.805 ± 0.016 | 31.79 |
| 24.3 ± 0.1 | 3.657 ± 0.015 | 24.25 |
| 25.3 ± 0.1 | 3.524 ± 0.014 | 26.02 |
| 25.8 ± 0.1 | 3.460 ± 0.013 | 32.29 |
| 26.5 ± 0.1 | 3.364 ± 0.013 | 71.65 |
| 27.2 ± 0.1 | 3.273 ± 0.012 | 20.34 |
| 27.7 ± 0.1 | 3.217 ± 0.011 | 8.97 |
| 28.4 ± 0.1 | 3.141 ± 0.011 | 11.26 |
| 29.7 ± 0.1 | 3.004 ± 0.010 | 19.01 |

### Example 8

### Preparation of OSI-906 Form H

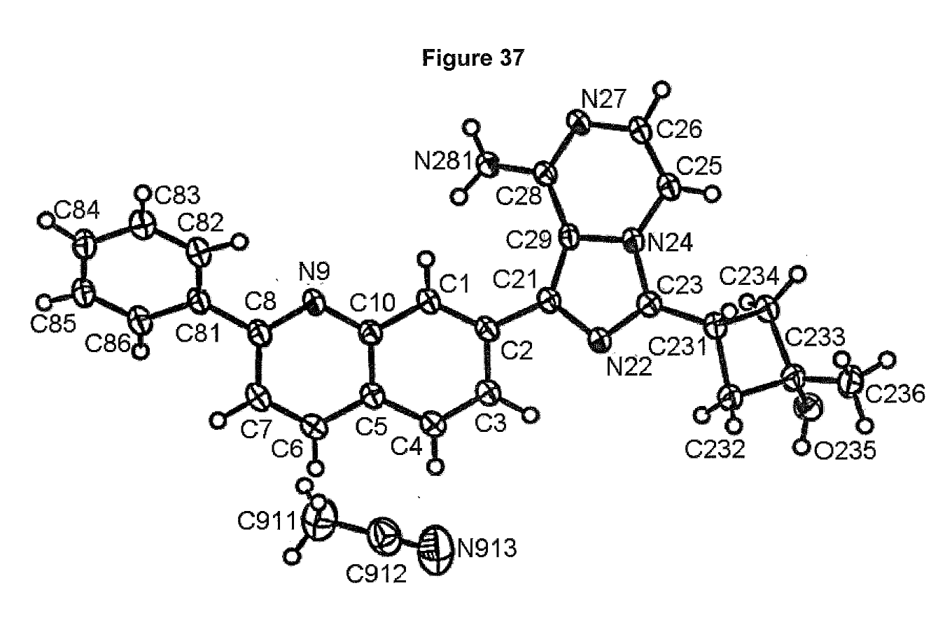

Crystals of OSI-906 were grown by slurrying in acetonitrile. The complete experimental details are provided in Table 14. The monoclinic cell parameters and calculated volume are: a=13.7274(3) Å, b=10.9853(3) Å, c=15.6016(4) Å, α=90.00°, β=96.5346(12)°, γ=90.00°, V=2337.43(10) Å³. The formula weight of the asymmetric unit in the crystal structure of OSI-906 was 462.56 g cm⁻³ with Z=4, resulting in a calculated density of 1.314 g cm⁻³. The space group was determined to be P2₁/n (No. 14). A summary of the crystal data and crystallographic data collection parameters are provided in 15. X-ray single crystallographic data was recorded and is reproduced in FIG. 37 and Tables 15-20. The XRPD of the sample is recorded and is reproduced in FIG. 10.

TABLE 14

| | | | Single |
|---|---|---|---|
| | | | Crystal |
| | | | Quality |
| Solvent | Conditions | Description | (Y/N) |
| MeOH | slow | fused, angular agglomerate; | — |
| | evaporation | several large angulars with | |
| | | drusy (B/R, ext.) | |
| acetone | slow | aggregate of 3 medium- | — |
| | evaporation | sized rhombohedrals | |
| | | (B/R, ext.); angulars, | |
| | | dendridics, small | |
| | | rhombohedrals (B/R, ext.) | |
| toluene | 1) Samples | extremely tiny particulates | N |
| | were first | initially (B/R); SE produced | |
| | slurried on a | tiny angular platys | |
| | rotating wheel | (B/R, ext.) | |
| | for | | |
| | approximately | | |
| | 3 days 2) slow | | |
| | evaporation | | |
| acetonitrile | 1) Samples | fairly large, thick platys and | Y |
| | were first | rhombohedrals; B/R, ext. | |
| | slurried on a | | |
| | rotating wheel | | |
| | for | | |
| | approximately | | |
| | 3 days 2) slow | | |
| | evaporation | | |
| tetrahydrofuran | 1) Samples | tiny particulates initially | — |
| | were first | (B/R); SE produced clear, | |
| | slurried on a | yellow film on bottom with | |
| | rotating wheel | “football” and “tear- | |
| | for | shaped” anhedrals | |
| | approximately | (B/R, ext.) | |
| | 3 days 2) slow | | |
| | evaporation | | |
| ethyl acetate | 1) Samples | tiny particulates initially | N |
| | were first | (B/R); SE produced dark | |
| | slurried on a | orange, anhedral flakes and | |
| | rotating wheel | small, fibrous dendridic | |
| | for | agglomerates and fibrous | |
| | approximately | spheres at liquid interface | |
| | 3 days 2) slow | (B/R, ext.); after 35 days: | |
| | evaporation | solids developed a red cast | |
| dichloromethane | Samples were | long aciculars; dendridic | — |
| | first slurried | agglomerate, anhedral, and | |
| | on a rotating | angular bands (B/R, ext.) | |
| | wheel for | | |
| | approximately | | |
| | 1 day | | |
| dioxane | Samples were | very tiny particulates | N |
| | first slurried | initially (B/R); SE produced | |
| | on a rotating | dendridics (B/R, ext.) and | |
| | wheel for | brittle glass (B/R) | |
| | approximately | | |
| | 1 day | | |
| 2,2,2- | slow | clear, yellow film | N |
| trifluoroethanol | evaporation | | |
| methyl ethyl | Samples were | amber liquid with a few | — |
| ketone | first slurried | long needles plus needle | |
| | on a rotating | dendridics with drusy | |
| | wheel for | (B/R, ext.); after 35 days: | |
| | approximately | dark purple liquid | |
| | 1 day | | |
| nitromethane | slow | small, yellow chunks and | Y |
| | evaporation | rhombohedrals (B/R, ext.) | |
| diethyl ether | Samples were | tiny particles | N |
| | first slurried | | |
| | on a rotating | | |
| | wheel for | | |
| | approximately | | |
| | 1 day | | |

TABLE 15

| formula | C₂₈H₂₆N₆O |
|---|---|
| formula weight | 462.56 |
| space group | P21/n (No. 14) |
| a, Å | 13.7274(3) |
| b, Å | 10.9853(3) |
| c, Å | 15.6016(4) |
| b, deg | 96.5346(12) |
| V, Å³ | 2337.43(10) |
| Z | 4 |
| d_{calc}, g cm⁻³ | 1.314 |
| crystal dimensions, mm | 0.38 × 0.19 × 0.11 |
| temperature, K | 150 |
| radiation (wavelength, Å) | Mo K_{α} (0.71073) |
| monochromator | graphite |
| linear abs coef, mm⁻¹ | 0.078 |
| absorption correction applied | empirical^{a} |
| transmission factors: min, max | 0.967; 0.991 |
| diffractometer | Nonius KappaCCD |
| h, k, l range | −17 to 17 −14 to 12 −20 to 20 |
| 2Θ range, deg | 4.20-55.03 |
| mosaicity, deg | 0.69 |
| programs used | SHELXTL |
| F₀₀₀ | 976 |
| Weighting | 1/[σ2(Fo2) + (0.1528P)2 + 0.0000P] |
| | where P = (Fo2 + 2Fc2)/3 |
| data collected | 16163 |
| unique data | 4065 |
| Rᵢₙₜ | 0.077 |
| data used in refinement | 4065 |
| cutoff used in R-factor | Fₒ² > 2.0σ(Fₒ²) |
| calculations | |
| data with />2.0σ(/) | 3142 |
| refined extinction coef | 0.019 |
| number of variables | 410 |
| largest shift/esd in final cycle | 0 |
| R(Fₒ) | 0.07 |
| R_{w}(Fₒ²) | 0.182 |
| goodness of fit | 1.009 |

^{a}Otwinowski Z. & Minor, W. Methods Enzymol., 276, 307, (1997).

TABLE 16

| Atom | x | y | z | U(Å²) |
|---|---|---|---|---|
| O(235) | 0.45582(13) | 0.17621(15) | 0.73973(11) | 0.0367(5) |
| N(9) | 0.75475(13) | 0.36584(18) | 0.15350(11) | 0.0297(5) |
| N(22) | 0.58719(13) | 0.36436(18) | 0.49507(11) | 0.0301(5) |
| N(24) | 0.70905(13) | 0.44637(17) | 0.58082(11) | 0.0278(5) |
| N(27) | 0.90429(13) | 0.49211(18) | 0.56815(11) | 0.0298(5) |
| N(281) | 0.89979(16) | 0.3904(2) | 0.44017(13) | 0.0330(6) |
| N(913) | 0.3277(2) | 0.3246(3) | 0.1863(2) | 0.0798(10) |
| C(1) | 0.71286(16) | 0.3665(2) | 0.29873(14) | 0.0286(6) |
| C(2) | 0.65820(16) | 0.3245(2) | 0.36138(14) | 0.0278(6) |
| C(3) | 0.58269(17) | 0.2370(2) | 0.33796(15) | 0.0316(6) |
| C(4) | 0.56546(17) | 0.1936(2) | 0.25577(15) | 0.0321(6) |
| C(5) | 0.62193(16) | 0.2351(2) | 0.19102(14) | 0.0295(6) |
| C(6) | 0.60481(18) | 0.1981(2) | 0.10425(15) | 0.0337(6) |
| C(7) | 0.65972(18) | 0.2458(2) | 0.04502(15) | 0.0340(6) |
| C(8) | 0.73575(17) | 0.3296(2) | 0.07163(14) | 0.0304(6) |
| C(10) | 0.69739(15) | 0.3218(2) | 0.21278(14) | 0.0274(6) |
| C(21) | 0.66866(16) | 0.3677(2) | 0.45088(14) | 0.0284(6) |
| C(23) | 0.61268(16) | 0.4109(2) | 0.57243(14) | 0.0298(6) |
| C(25) | 0.76486(17) | 0.5047(2) | 0.64890(14) | 0.0304(6) |
| C(26) | 0.85891(17) | 0.5278(2) | 0.63894(14) | 0.0308(6) |
| C(28) | 0.85124(15) | 0.4351(2) | 0.50426(13) | 0.0270(6) |
| C(29) | 0.74743(16) | 0.4168(2) | 0.50429(13) | 0.0268(6) |
| C(81) | 0.79489(16) | 0.3846(2) | 0.00743(14) | 0.0295(6) |
| C(82) | 0.84029(18) | 0.4964(2) | 0.02318(15) | 0.0361(7) |
| C(83) | 0.89028(18) | 0.5513(3) | −0.03799(15) | 0.0388(7) |
| C(84) | 0.89556(18) | 0.4953(2) | −0.11762(15) | 0.0367(7) |
| C(85) | 0.85130(18) | 0.3836(2) | −0.13413(15) | 0.0366(7) |
| C(86) | 0.80125(18) | 0.3282(2) | −0.07252(15) | 0.0341(7) |
| C(231) | 0.55116(16) | 0.4185(2) | 0.64462(14) | 0.0307(6) |
| C(232) | 0.45160(17) | 0.3503(2) | 0.63263(15) | 0.0318(6) |
| C(233) | 0.47229(16) | 0.3028(2) | 0.72605(14) | 0.0315(6) |
| C(234) | 0.58158(17) | 0.3347(2) | 0.72423(15) | 0.0343(7) |
| C(236) | 0.4239(2) | 0.3781(3) | 0.79075(17) | 0.0403(8) |
| C(911) | 0.4000(3) | 0.4005(3) | 0.0502(2) | 0.0652(10) |
| C(912) | 0.3594(2) | 0.3577(3) | 0.1265(2) | 0.0540(9) |
| H(1) | 0.7604(18) | 0.437(2) | 0.3109(15) | 0.029(6)* |
| H(3) | 0.5395(19) | 0.206(2) | 0.3823(17) | 0.038(7)* |
| H(4) | 0.513(2) | 0.135(2) | 0.2421(18) | 0.042(7)* |
| H(6) | 0.554(2) | 0.141(2) | 0.0895(17) | 0.038(7)* |
| H(7) | 0.6438(19) | 0.224(2) | −0.0159(19) | 0.044(7)* |
| H(25) | 0.7310(19) | 0.526(2) | 0.6980(17) | 0.036(7)* |
| H(26) | 0.9022(18) | 0.574(2) | 0.6834(16) | 0.031(6)* |
| H(82) | 0.831(2) | 0.536(3) | 0.077(2) | 0.053(8)* |
| H(83) | 0.9172(19) | 0.631(3) | −0.0279(17) | 0.040(7)* |
| H(84) | 0.936(2) | 0.537(2) | −0.1568(17) | 0.041(7)* |
| H(85) | 0.863(3) | 0.339(3) | −0.185(3) | 0.082(11)* |
| H(86) | 0.7746(17) | 0.250(2) | −0.0811(16) | 0.033(7)* |
| H(231) | 0.5454(19) | 0.502(3) | 0.6632(17) | 0.041(7)* |
| H(235) | 0.401(3) | 0.155(3) | 0.713(2) | 0.076(12)* |
| H(23A) | 0.6152(19) | 0.370(2) | 0.7779(18) | 0.039(7)* |
| H(23C) | 0.4372(19) | 0.467(3) | 0.7841(17) | 0.041(7)* |
| H(23D) | 0.450(2) | 0.357(3) | 0.849(2) | 0.044(7)* |
| H(23E) | 0.352(2) | 0.365(2) | 0.7879(18) | 0.047(8)* |
| H(23F) | 0.392(2) | 0.401(3) | 0.6198(18) | 0.048(8)* |
| H(23G) | 0.4534(19) | 0.283(2) | 0.5934(17) | 0.038(7)* |
| H(28A) | 0.956(2) | 0.423(3) | 0.4348(18) | 0.042(8)* |
| H(28B) | 0.867(2) | 0.365(3) | 0.389(2) | 0.044(7)* |
| H(91A) | 0.401 | 0.49 | 0.05 | 0.098 |
| H(91B) | 0.359 | 0.372 | −0.002 | 0.098 |
| H(91C) | 0.467 | 0.369 | 0.05 | 0.098 |

Starred atoms were refined isotropically
U_{eq} = (⅓)ΣᵢΣⱼ Uᵢⱼa*ᵢa*ⱼaᵢ.aⱼ
Hydrogen atoms are included in calculation of structure factors but not refined

TABLE 17

| Atom 1 | Atom 2 | Distance | Atom 1 | Atom 2 | Distance |
|---|---|---|---|---|---|
| O235 | C233 | 1.429(3) | C25 | C26 | 1.342(3) |
| O235 | H235 | 0.85(4) | C25 | H25 | 0.97(3) |
| N9 | C8 | 1.335(3) | C26 | H26 | 1.00(3) |
| N9 | C10 | 1.369(3) | C28 | C29 | 1.439(3) |
| N22 | C23 | 1.321(3) | C81 | C82 | 1.386(3) |
| N22 | C21 | 1.379(3) | C81 | C86 | 1.405(3) |
| N24 | C23 | 1.371(3) | C82 | C83 | 1.376(3) |
| N24 | C25 | 1.393(3) | C82 | H82 | 0.97(3) |
| N24 | C29 | 1.397(3) | C83 | C84 | 1.395(3) |
| N27 | C28 | 1.323(3) | C83 | H83 | 0.96(3) |
| N27 | C26 | 1.385(3) | C84 | C85 | 1.380(4) |
| N281 | C28 | 1.355(3) | C84 | H84 | 0.99(3) |
| N281 | H28A | 0.87(3) | C85 | C86 | 1.385(3) |
| N281 | H28B | 0.92(3) | C85 | H85 | 0.97(4) |
| N913 | C912 | 1.134(4) | C86 | H86 | 0.94(3) |
| C1 | C2 | 1.378(3) | C231 | C232 | 1.551(3) |
| C1 | C10 | 1.421(3) | C231 | C234 | 1.564(3) |
| C1 | H1 | 1.02(2) | C231 | H231 | 0.97(3) |
| C2 | C3 | 1.430(3) | C232 | C233 | 1.544(3) |
| C2 | C21 | 1.466(3) | C232 | H23F | 0.99(3) |
| C3 | C4 | 1.363(3) | C232 | H23G | 0.96(3) |
| C3 | H3 | 1.02(3) | C233 | C236 | 1.515(3) |
| C4 | C5 | 1.417(3) | C233 | C234 | 1.544(3) |
| C4 | H4 | 0.97(3) | C234 | H23A | 0.99(3) |
| C5 | C6 | 1.407(3) | C234 | H23B | 1.04(3) |
| C5 | C10 | 1.419(3) | C236 | H23C | 1.00(3) |
| C6 | C7 | 1.362(3) | C236 | H23D | 0.97(3) |
| C6 | H6 | 0.95(3) | C236 | H23E | 0.99(3) |
| C7 | C8 | 1.418(3) | C911 | C912 | 1.449(5) |
| C7 | H7 | 0.98(3) | C911 | H91A | 0.98 |
| C8 | C81 | 1.488(3) | C911 | H91B | 0.98 |
| C21 | C29 | 1.396(3) | C911 | H91C | 0.98 |
| C23 | C231 | 1.485(3) | | | |

Numbers in parentheses are estimated standard deviations in the least significant digits.

TABLE 18

| Atom 1 | Atom 2 | Atom 3 | Angle |
|---|---|---|---|
| C233 | O235 | H235 | 110(2) |
| C8 | N9 | C10 | 118.43(19) |
| C23 | N22 | C21 | 107.50(18) |
| C23 | N24 | C25 | 130.13(19) |
| C23 | N24 | C29 | 107.81(18) |
| C25 | N24 | C29 | 122.04(18) |
| C28 | N27 | C26 | 118.42(18) |
| C28 | N281 | H28A | 116.4(19) |
| C28 | N281 | H28B | 120.9(17) |
| H28A | N281 | H28B | 114(2) |
| C2 | C1 | C10 | 121.3(2) |
| C2 | C1 | H1 | 120.8(14) |
| C10 | C1 | H1 | 117.6(14) |
| C1 | C2 | C3 | 118.8(2) |
| C1 | C2 | C21 | 124.4(2) |
| C3 | C2 | C21 | 116.8(2) |
| C4 | C3 | C2 | 121.2(2) |
| C4 | C3 | H3 | 118.0(15) |
| C2 | C3 | H3 | 120.8(15) |
| C3 | C4 | C5 | 120.5(2) |
| C3 | C4 | H4 | 119.0(16) |
| C5 | C4 | H4 | 120.5(16) |
| C6 | C5 | C4 | 123.2(2) |
| C6 | C5 | C10 | 117.5(2) |
| C4 | C5 | C10 | 119.3(2) |
| C7 | C6 | C5 | 119.8(2) |
| C7 | C6 | H6 | 122.6(16) |
| C5 | C6 | H6 | 117.6(16) |
| C6 | C7 | C8 | 119.8(2) |
| C6 | C7 | H7 | 119.0(15) |
| C8 | C7 | H7 | 121.1(15) |
| N9 | C8 | C7 | 122.1(2) |
| N9 | C8 | C81 | 117.4(2) |
| C7 | C8 | C81 | 120.4(2) |
| N9 | C10 | C5 | 122.38(19) |
| N9 | C10 | C1 | 118.6(2) |
| C5 | C10 | C1 | 119.0(2) |
| N22 | C21 | C29 | 109.21(19) |
| N22 | C21 | C2 | 118.04(19) |
| C29 | C21 | C2 | 132.7(2) |
| N22 | C23 | N24 | 110.47(19) |
| N22 | C23 | C231 | 126.9(2) |
| N24 | C23 | C231 | 122.52(19) |
| C26 | C25 | N24 | 116.8(2) |
| C26 | C25 | H25 | 126.8(15) |
| N24 | C25 | H25 | 116.3(15) |
| C25 | C26 | N27 | 124.4(2) |
| C25 | C26 | H26 | 121.4(14) |
| H23A | C234 | H23B | 108(2) |
| C233 | C236 | H23C | 111.4(15) |
| C233 | C236 | H23D | 110.8(17) |
| H23C | C236 | H23D | 106(2) |
| C233 | C236 | H23E | 113.7(17) |
| H23C | C236 | H23E | 109(2) |
| H23D | C236 | H23E | 105(2) |
| N27 | C26 | H26 | 114.3(14) |
| N27 | C28 | N281 | 116.91(19) |
| N27 | C28 | C29 | 121.74(19) |
| N281 | C28 | C29 | 121.3(2) |
| C21 | C29 | N24 | 104.96(18) |
| C21 | C29 | C28 | 138.9(2) |
| N24 | C29 | C28 | 115.93(18) |
| C82 | C81 | C86 | 118.4(2) |
| C82 | C81 | C8 | 120.6(2) |
| C86 | C81 | C8 | 120.8(2) |
| C83 | C82 | C81 | 121.0(2) |
| C83 | C82 | H82 | 122.0(18) |
| C81 | C82 | H82 | 116.8(18) |
| C82 | C83 | C84 | 120.3(2) |
| C82 | C83 | H83 | 120.1(16) |
| C84 | C83 | H83 | 119.4(16) |
| C85 | C84 | C83 | 119.4(2) |
| C85 | C84 | H84 | 124.6(15) |
| C83 | C84 | H84 | 115.8(15) |
| C84 | C85 | C86 | 120.3(2) |
| C84 | C85 | H85 | 120(2) |
| C86 | C85 | H85 | 119(2) |
| C85 | C86 | C81 | 120.5(2) |
| C85 | C86 | H86 | 121.4(15) |
| C81 | C86 | H86 | 118.0(15) |
| C23 | C231 | C232 | 116.79(19) |
| C23 | C231 | C234 | 116.5(2) |
| C232 | C231 | C234 | 87.90(17) |
| C23 | C231 | H231 | 110.6(16) |
| C232 | C231 | H231 | 113.2(16) |
| C234 | C231 | H231 | 110.1(15) |
| C233 | C232 | C231 | 89.11(17) |
| C233 | C232 | H23F | 115.9(16) |
| C231 | C232 | H23F | 116.9(16) |
| C233 | C232 | H23G | 108.9(16) |
| C231 | C232 | H23G | 111.3(15) |
| H23F | C232 | H23G | 113(2) |
| O235 | C233 | C236 | 110.04(19) |
| O235 | C233 | C232 | 117.0(2) |
| C236 | C233 | C232 | 113.4(2) |
| O235 | C233 | C234 | 113.21(19) |
| C236 | C233 | C234 | 112.9(2) |
| C232 | C233 | C234 | 88.90(17) |
| C233 | C234 | C231 | 88.62(17) |
| C233 | C234 | H23A | 115.8(15) |
| C231 | C234 | H23A | 119.9(16) |
| C233 | C234 | H23B | 110.6(13) |
| C231 | C234 | H23B | 112.5(13) |
| C912 | C911 | H91A | 109.5 |
| C912 | C911 | H91B | 109.5 |
| H91A | C911 | H91B | 109.5 |
| C912 | C911 | H91C | 109.5 |
| H91A | C911 | H91C | 109.5 |
| H91B | C911 | H91C | 109.5 |
| N913 | C912 | C911 | 179.8(4) |

Numbers in parentheses are estimated standard deviations in the least significant digits.

TABLE 19

| D | H | A | D-H | A-H | D-A | D-H-A |
|---|---|---|---|---|---|---|
| O(235) | H(235) | N(9) | 0.85(4) | 2.13(4) | 2.966(4) | 169(3) |
| N(281) | H(28A) | N(27) | 0.86(3) | 2.14(3) | 2.999(4) | 176(3) |

Numbers in parentheses are estimated standard deviations in the least significant digits.

TABLE 20

| Atom 1 | Atom 2 | Atom 3 | Atom 4 | Angle | |
|---|---|---|---|---|---|
| C(10) | N(9) | C(8) | C(7) | −1.21 | (0.33) |
| C(10) | N(9) | C(8) | C(81) | 176.23 | (0.19) |
| C(8) | N(9) | C(10) | C(1) | −175.69 | (0.20) |
| C(8) | N(9) | C(10) | C(5) | 3.03 | (0.32) |
| C(23) | N(22) | C(21) | C(2) | −177.91 | (0.19) |
| C(23) | N(22) | C(21) | C(29) | 0.91 | (0.25) |
| C(21) | N(22) | C(23) | N(24) | 0.56 | (0.25) |
| C(21) | N(22) | C(23) | C(231) | −175.54 | (0.21) |
| C(25) | N(24) | C(23) | N(22) | 176.50 | (0.21) |
| C(25) | N(24) | C(23) | C(231) | −7.21 | (0.35) |
| C(29) | N(24) | C(23) | N(22) | −1.80 | (0.25) |
| C(29) | N(24) | C(23) | C(231) | 174.49 | (0.20) |
| C(23) | N(24) | C(25) | C(26) | 179.69 | (0.22) |
| C(29) | N(24) | C(25) | C(26) | −2.22 | (0.31) |
| C(23) | N(24) | C(29) | C(21) | 2.25 | (0.23) |
| C(23) | N(24) | C(29) | C(28) | −173.46 | (0.19) |
| C(25) | N(24) | C(29) | C(21) | −176.22 | (0.19) |
| C(25) | N(24) | C(29) | C(28) | 8.08 | (0.30) |
| C(28) | N(27) | C(26) | C(25) | 2.49 | (0.33) |
| C(26) | N(27) | C(28) | N(281) | −173.42 | (0.20) |
| C(26) | N(27) | C(28) | C(29) | 4.10 | (0.32) |
| C(10) | C(1) | C(2) | C(3) | −1.93 | (0.33) |
| C(10) | C(1) | C(2) | C(21) | −178.86 | (0.21) |
| C(2) | C(1) | C(10) | N(9) | −179.21 | (0.20) |
| C(2) | C(1) | C(10) | C(5) | 2.02 | (0.32) |
| C(1) | C(2) | C(3) | C(4) | 1.13 | (0.34) |
| C(21) | C(2) | C(3) | C(4) | 178.30 | (0.21) |
| C(1) | C(2) | C(21) | N(22) | 150.82 | (0.22) |
| C(1) | C(2) | C(21) | C(29) | −27.67 | (0.39) |
| C(3) | C(2) | C(21) | N(22) | −26.17 | (0.30) |
| C(3) | C(2) | C(21) | C(29) | 155.34 | (0.24) |
| C(2) | C(3) | C(4) | C(5) | −0.44 | (0.35) |
| C(3) | C(4) | C(5) | C(6) | −176.98 | (0.22) |
| C(3) | C(4) | C(5) | C(10) | 0.53 | (0.34) |
| C(4) | C(5) | C(6) | C(7) | 177.35 | (0.22) |
| C(10) | C(5) | C(6) | C(7) | −0.20 | (0.33) |
| C(4) | C(5) | C(10) | N(9) | −179.98 | (0.41) |
| C(4) | C(5) | C(10) | C(1) | −1.28 | (0.32) |
| C(6) | C(5) | C(10) | N(9) | −2.34 | (0.32) |
| C(6) | C(5) | C(10) | C(1) | 176.37 | (0.20) |
| C(5) | C(6) | C(7) | C(8) | 1.92 | (0.34) |
| C(6) | C(7) | C(8) | N(9) | −1.27 | (0.35) |
| C(6) | C(7) | C(8) | C(81) | −178.63 | (0.21) |
| N(9) | C(8) | C(81) | C(82) | −22.92 | (0.32) |
| N(9) | C(8) | C(81) | C(86) | 160.89 | (0.21) |
| C(7) | C(8) | C(81) | C(82) | 154.56 | (0.22) |
| C(7) | C(8) | C(81) | C(86) | −21.63 | (0.33) |
| N(22) | C(21) | C(29) | N(24) | −1.95 | (0.24) |
| N(22) | C(21) | C(29) | C(28) | 172.17 | (0.26) |
| C(2) | C(21) | C(29) | N(24) | 176.64 | (0.23) |
| C(2) | C(21) | C(29) | C(28) | −9.24 | (0.47) |
| N(22) | C(23) | C(231) | C(232) | 8.51 | (0.33) |
| N(22) | C(23) | C(231) | C(234) | 110.38 | (0.26) |
| N(24) | C(23) | C(231) | C(232) | −167.15 | (0.20) |
| N(24) | C(23) | C(231) | C(234) | −65.29 | (0.28) |
| N(24) | C(25) | C(26) | N(27) | −3.44 | (0.33) |
| N(27) | C(28) | C(29) | N(24) | −9.09 | (0.31) |
| N(27) | C(28) | C(29) | C(21) | 177.23 | (0.26) |
| N(281) | C(28) | C(29) | N(24) | 168.32 | (0.20) |
| N(281) | C(28) | C(29) | C(21) | −5.36 | (0.42) |
| C(8) | C(81) | C(82) | C(83) | −176.11 | (0.22) |
| C(86) | C(81) | C(82) | C(83) | 0.16 | (0.35) |
| C(8) | C(81) | C(86) | C(85) | 175.92 | (0.22) |
| C(82) | C(81) | C(86) | C(85) | −0.35 | (0.34) |
| C(81) | C(82) | C(83) | C(84) | 0.43 | (0.38) |
| C(82) | C(83) | C(84) | C(85) | −0.84 | (0.38) |
| C(83) | C(84) | C(85) | C(86) | 0.65 | (0.36) |
| C(84) | C(85) | C(86) | C(81) | −0.06 | (0.40) |
| C(23) | C(231) | C(232) | C(233) | 136.24 | (0.20) |
| C(234) | C(231) | C(232) | C(233) | 17.48 | (0.16) |
| C(23) | C(231) | C(234) | C(233) | −136.53 | (0.19) |
| C(232) | C(231) | C(234) | C(233) | −17.48 | (0.16) |
| C(231) | C(232) | C(233) | O(235) | −133.36 | (0.19) |
| C(231) | C(232) | C(233) | C(234) | −17.71 | (0.16) |
| C(231) | C(232) | C(233) | C(236) | 96.83 | (0.21) |
| O(235) | C(233) | C(234) | C(231) | 136.68 | (0.18) |
| C(232) | C(233) | C(234) | C(231) | 17.56 | (0.16) |
| C(236) | C(233) | C(234) | C(231) | −97.44 | (0.21) |

Numbers in parentheses are estimated standard deviations in the least significant digits.

### Example 9

### Preparation of OSI-906 Form I

To a glass flask was added 1.0 g of OSI-906 and 10 mL sec-butanol. Agitated solution and heated to reflux for 30 minutes. Cooled resultant slurry to ambient. The fine solid was collected by filtration and was washed with 1 ml sec-butanol. The solid was dried at 45° C. under vacuum to give 795.0 mg of Form I.

### Thermodynamic Stability Experiments

Gravimetric Moisture Sorption:

Gravimetric moisture sorption experiments were carried out on selected materials by first drying the sample at 40% RH and 25° C. until an equilibrium weight was reached or for a maximum of four hours. The sample was then subjected to an isothermal (25° C.) adsorption scan from 40 to 90% RH in steps of 10%. The sample was allowed to equilibrate to an asymptotic weight at each point for a maximum of four hours. Following adsorption, a desorption scan from 85 to 0% RH (at 25° C.) was run in steps of −10% again allowing a maximum of four hours for equilibration to an asymptotic weight. An adsorption scan was then performed from 0% RH to 40% RH in steps of +10% RH. The sample was then dried for 1-2 hours at 60° C. and the resulting solid analyzed by XRPD.

Solid-State Stability:

Approximately 50 mg of Form A or Forms C+D were weighed to individual 8 mL vials and placed uncapped in the following storage conditions: 40° C. under vacuum, 80° C. under vacuum, desiccant, 25° C./60% RH and 40° C./75% RH. After 24 hours and seven days of equilibration the solids were analyzed by XRPD and ¹H-NMR. (Table 21).

Grinding Experiments:

Approximately 50 mg of Form A was either ground in a mortar and pestle for five minutes or in a ball mill for 2 minutes at 10 Hz. Resulting materials were analyzed by XRPD to confirm the solid form and then transferred to 8 mL vials. The vials were stored uncapped at 80° C. under vacuum for seven days and then analyzed by XRPD and ¹H-NMR. (Table 21).

TABLE 21

| Initial | Mass | | Duration | XRPD |
|---|---|---|---|---|
| Form | (mg) | Storage condition | (days) | (Form) |
| A | — | — | Control | A |
| | 51.6 | Desiccant | 1 | A |
| | | | 7 | A |
| | 52.4 | 40° C. under vacuum | 1 | A |
| | | | 7 | A |
| | 51.6 | 80° C. under vacuum | 1 | A |
| | | | 7 | A |
| | 50.0 | Mortar/Pestle 5 min, 80° C. under | 7 | A |
| | | vacuum | | |
| | 50.0 | Ball mill 5 min, 80° C. under | 7 | A |
| | | vacuum | | |
| | 50.0 | 25° C./60% RH | 1 | A |
| | | | 7 | A |
| | 50.8 | 40° C./75% RH | 1 | A |
| | | | 7 | A |
| C + D | — | — | Control | C + D |
| | 46.8 | Desiccant | 1 | C + D |
| | | | 7 | C + D |
| | 52.6 | 40° C. under vacuum | 1 | C |
| | 52.0 | | 7 | C |
| | 50.4 | 80° C. under vacuum | 1 | C |
| | 47.0 | | 7 | C |
| | 52.5 | 25° C./60% RH | 1 | C + D |
| | | | 7 | C + D |
| | 50.6 | 40° C./75% RH | 1 | C + D |
| | | | 7 | C + D |

Slurry Experiments:

Approximately 20-50 mg of select crystalline forms were weighed to individual 8 mL vials equipped with a magnetic stir bar. Either THF, water, EtOH, (80:20) EtOH:Water or IPA was added to obtain a free flowing slurry. After 3, 5, 7 and 11 days of equilibration at 50° C. or ambient temperature, solid from each slurry was recovered by centrifuge filtration through 0.45 μm nylon filters. The isolated solids were analyzed by XRPD to check for form conversion. Select materials were then dried overnight under vacuum at ambient temperature and analyzed by ¹H-NMR to determine residual solvent content. (Table 22).

TABLE 22

| | | | | | | | | ¹H-NMR |
|---|---|---|---|---|---|---|---|---|
| Initial | Mass | Sol- | Vol | Temp | Inter- | | XRPD | Solvent, |
| Form | (mg) | vent | (mL) | ° C. | val | | (Form) | ppm |
| A | 31.6 | THF | 0.25 | 50 | 5 | d | A | 5749, |
| | | | | | | | | THF |
| | 29.5 | | 0.25 | RT | 11 | d | A | — |
| C + D | 31.4 | | 0.25 | 50 | 5 | d | A | 3477, |
| | | | | | | | | THF |
| | 28.7 | | 0.25 | RT | 11 | d | A | — |
| C | 14.0 + | | 0.25 | RT | 11 | d | A | — |
| | 6.6 | | | | | | | |
| A | 51.4 | Water | 0.50 | 50 | 3 | d | A | |
| | 33.1 | | 0.25 | 50 | 5 | d | A | — |
| | 51.4 | | 0.50 | 50 | 7 | d | D | — |
| | 33.1 | | 0.25 | 50 | 11 | d | D | — |
| | 53.2 | | 0.50 | RT | 3 | d | A | — |
| | | | | RT | 7 | d | D | — |
| | 29.2 | | 0.25 | RT | 11 | d | D | ND |
| C + D | 24.7 | | 0.25 | 50 | 5 | d | C + D | — |
| | | | | | 11 | d | C + D | — |
| | 28.8 | | 0.25 | RT | 11 | d | C + D | — |
| C | 20.1 | | 0.25 | RT | 11 | d | C | — |
| A | 31.8 | EtOH | 0.25 | 50 | 5 | d | A | 1420, |
| | | | | | | | | EtOH |
| | 28.1 | | | RT | 11 | d | C | 1744, IPA |
| C + D | 27.0 | | | 50 | 5 | d | A + E* | — |
| | | | | | 11 | d | A | 1565, |
| | | | | | | | | EtOH |
| | 29.1 | | | RT | 11 | d | C | 1774, IPA |
| C | 19.8 | | | RT | 11 | d | C | — |
| A | 32.5 | 8:2 | 0.25 | 50 | 5 | d | C | — |
| | | EtOH: | | | 11 | d | C | ND |
| | 29.6 | Water | | RT | 11 | d | C | ND |
| C + D | 27.4 | | | 50 | 5 | d | C + D | — |
| | | | | | 11 | d | C | — |
| | 31.6 | | | RT | 11 | d | C | — |
| C | 15.4 + | | | RT | 11 | d | C | — |
| | 3.6 | | | | | | | |

— not determined

### Refluxing Experiment:

Form Stability—Approximately 40-100 mg of select OSI-906 crystalline forms were weighed to a 4-mL or 8-mL vials equipped with a magnetic stir bar. To each container, 1.2 mL of EtOH or IPA was added and the resulting slurry heated to 80-83° C. After three hours of stirring, the solutions were cooled to room temperature at 10° C./hr. The resulting slurries were allowed to equilibrate for up to three days at ambient temperature and the solids isolated by centrifuge filtration. Recovered materials were analyzed by XRPD to determine the crystalline form (Table 23).

TABLE 23

| | | | | | XRPD | XRPD | XRPD | XRPD |
|---|---|---|---|---|---|---|---|---|
| | | | | | after | 24 h | 48 h | 3 d |
| | Mass | | Vol | Temp | cooling | at RT | at RT | at RT |
| Initial Form | (mg) | Solvent | (ml) | ° C. | (Form) | (Form) | (Form) | (Form) |
| C + D | 103.0 | EtOH | 1.2 | 83 | A | A | I | — |
| A + C + D + I | 10.1, 10-20 | IPA | 1.2 | 80 | — | — | — | A |

— not determined

### Thermodynamic Stability (Form A)

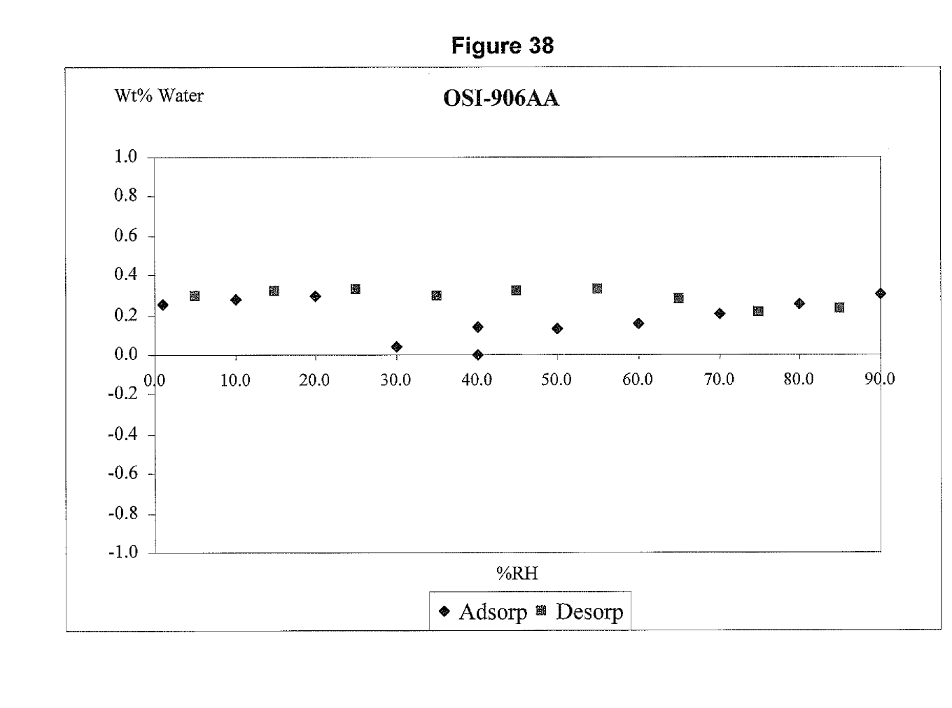

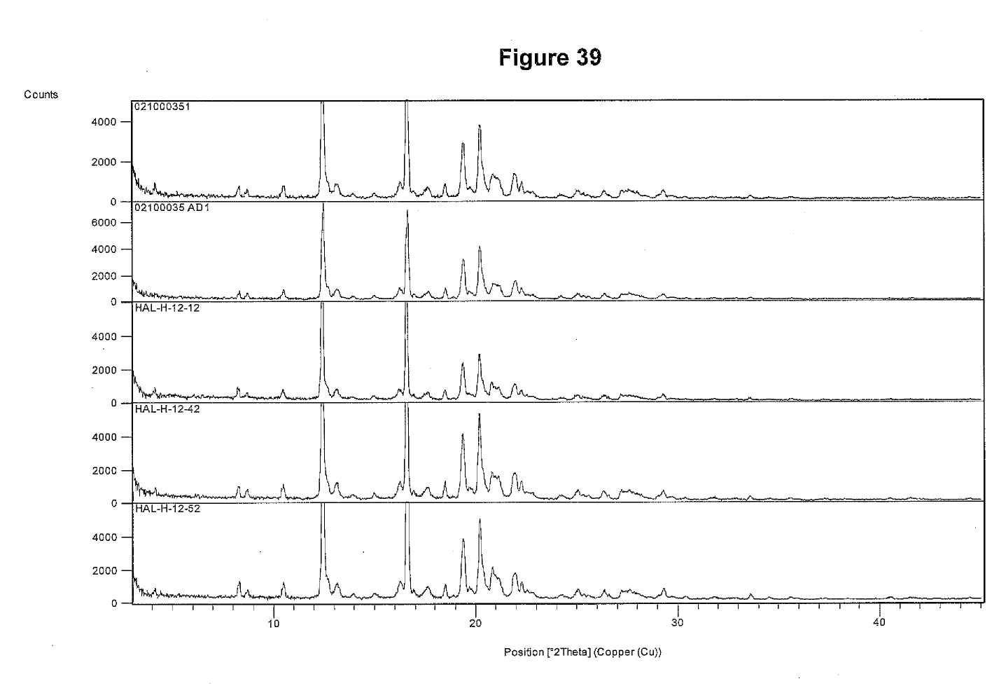

Form A was determined to be non-hygroscopic by gravimetric moisture sorption analysis. The solid form adsorbed 0.2 wt % water at 60% RH and 0.3 wt % water at 90% RH (See FIG. 38). Following the experiment, XRPD analysis of the dried solid afforded a diffraction pattern consistent with the initial form (See FIG. 39).

To assess the stability of Form A, the solid form was stored at different environmental conditions as described herein. Approximately 50 mg of Form A was weighed to 8 mL vials and placed uncapped in the following storage conditions: 40° C. under vacuum, 80° C. under vacuum, desiccant, 25° C./60% RH and 40° C./75% RH. After 24 hours and seven days of equilibration the solids were analyzed by XRPD. (See Table 21).

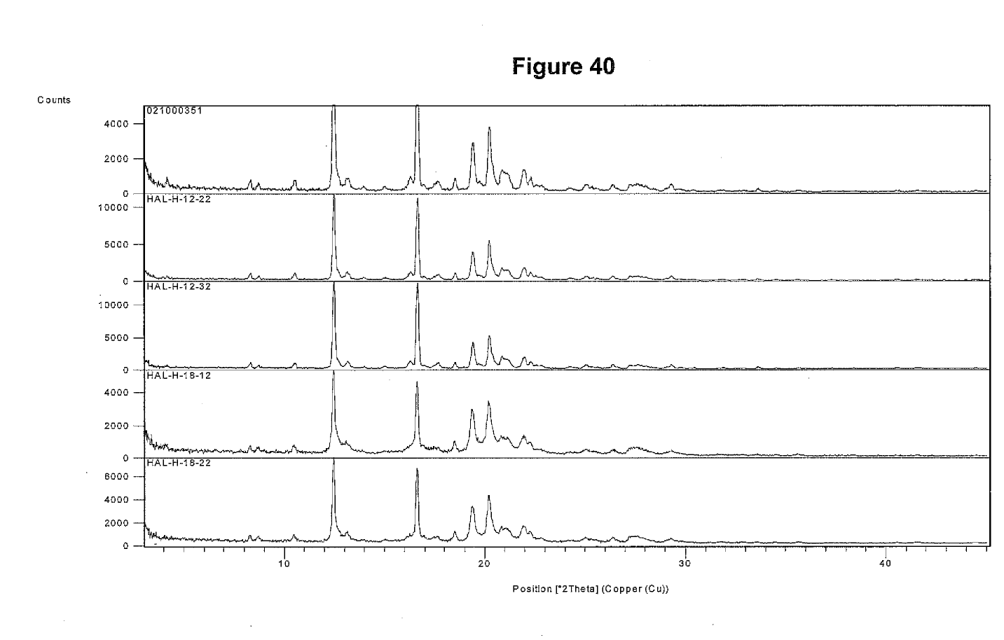

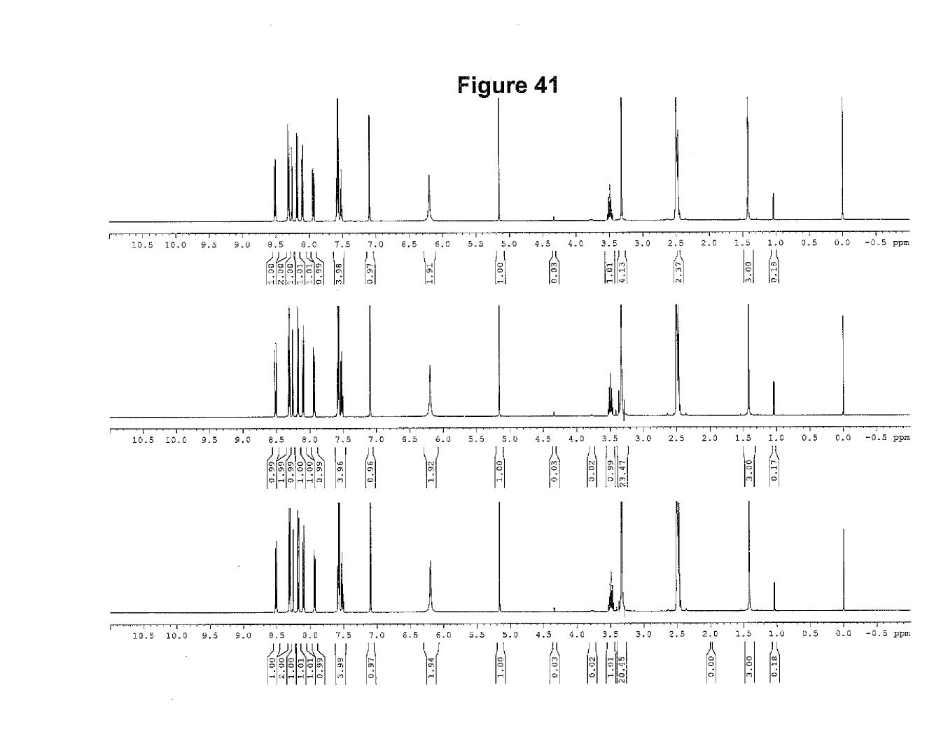

Form A exhibited stability following 24 hours and seven days of storage at 40° C. under vacuum, 80° C. under vacuum, 25° C./60% RH, 40° C./75% RH and under desiccant conditions. Representative XRPD patterns obtained following the time points are presented in FIG. 39 and FIG. 40. ¹H-NMR spectra of the samples obtained following drying for seven days at 40° C. under vacuum and 80° C. under vacuum showed no significant reduction in the levels of IPA (See FIG. 41).

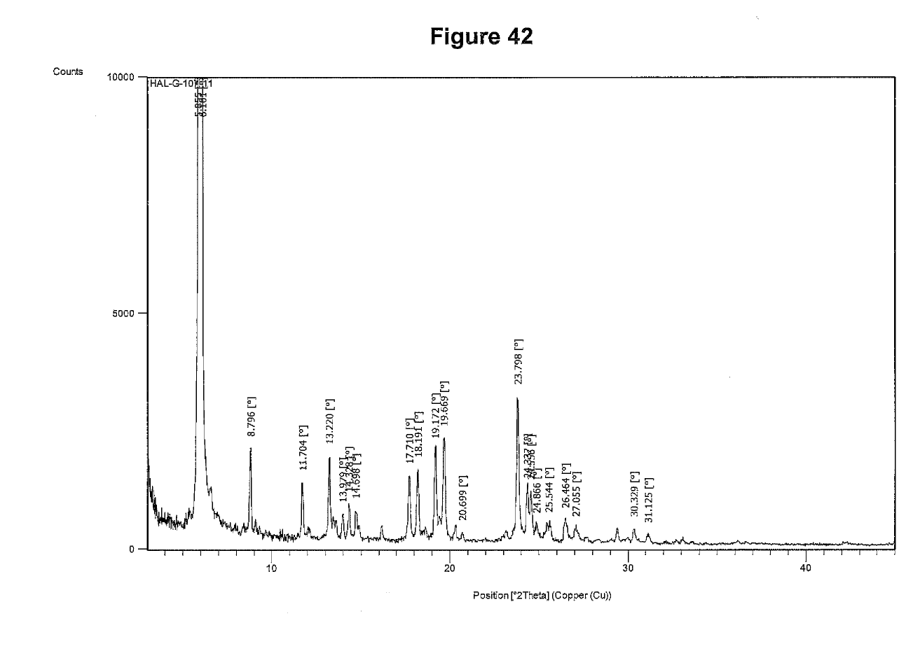

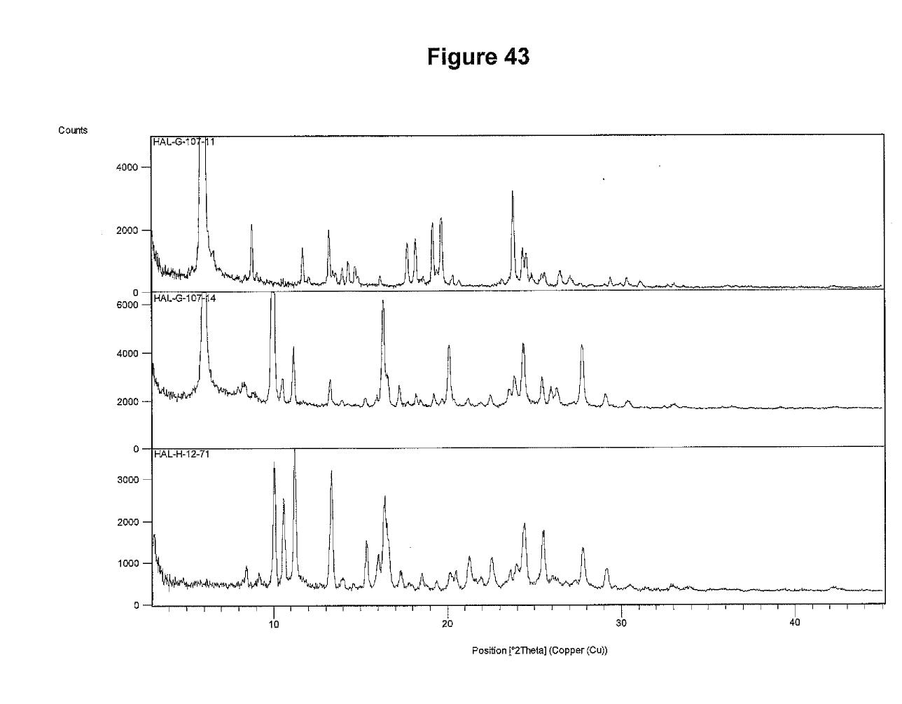

In an effort to better understand the nature of the IPA retention, crystallizations were performed to generate Form F, previously identified as an IPA solvate. These experiments were observed to be successful as shown in Table 24, FIG. 42, FIG. 43, and FIG. 7.

TABLE 24

| | IPA | | | | Recov- | | ¹H-NMR |
|---|---|---|---|---|---|---|---|
| Mass | volume | Temp | | Isola- | ery | XRPD | solvent, |
| (mg) | (mL) | ° C. | Cooling | tion | (mg) | (Form) | wt % |
| 35.5 | 6.4 | 70 | Freez- | Filter | 26.3 | F | IPA, 20.8 |
| 211.0 | 36.0 | | er, −15° | | 156.3 | F | IPA, 19.1 |
| 36.0 | 6.4 | | C. | | 25.1 | F | — |

— not determined

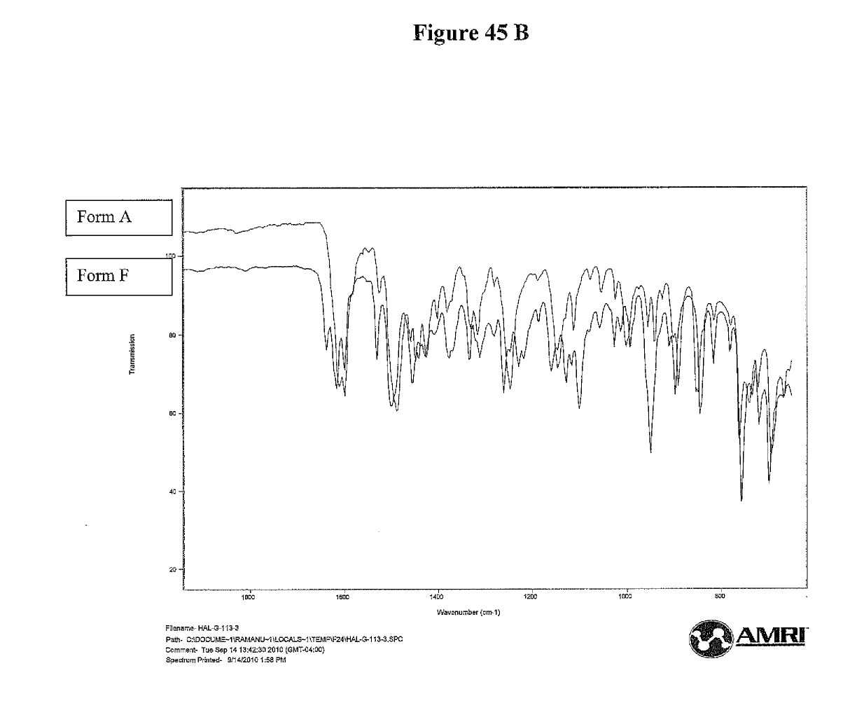

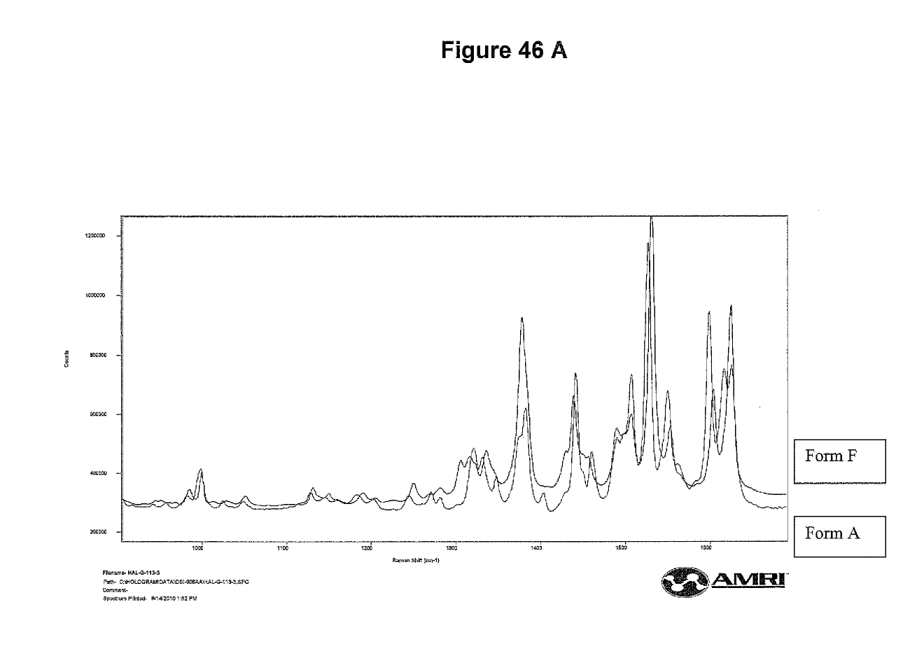

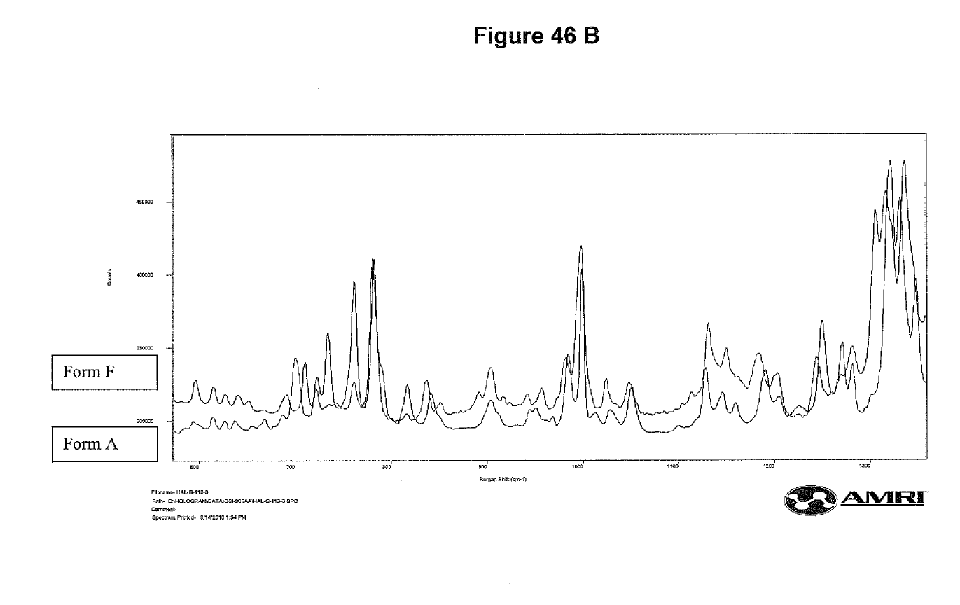

The ¹H-NMR spectrum of Form F showed approximately 20.8 wt % IPA which is comparable to the theoretical IPA content (22.2%) of a di-IPA solvate of OSI-906. Form F was analyzed by Raman and FTIR and spectra compared to corresponding data obtained for Form A. As shown in FIG. 45 and FIG. 46, several major spectral bands signature of Form F were not observed in the data obtained for Form A suggesting that the IPA retained is not solvated or the concentration is below a detectable limit. Form F was determined to be unstable in the solid state converting to a mixture of Forms C+F after eight days of storage in a sealed vial at ambient temperature (See FIG. 43).

### Thermodynamic Stability (Form C)

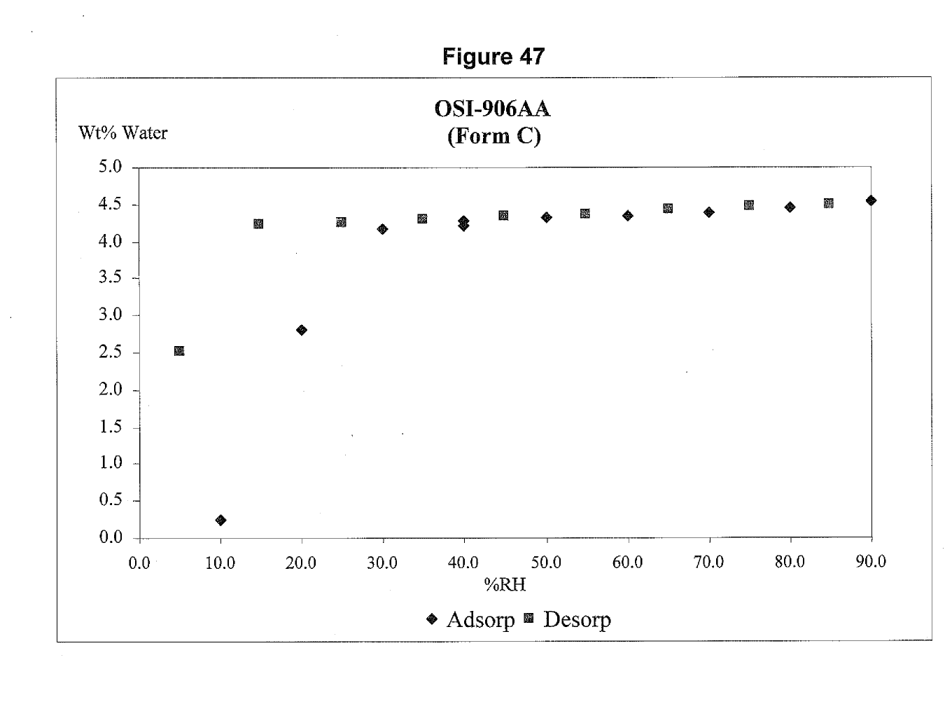

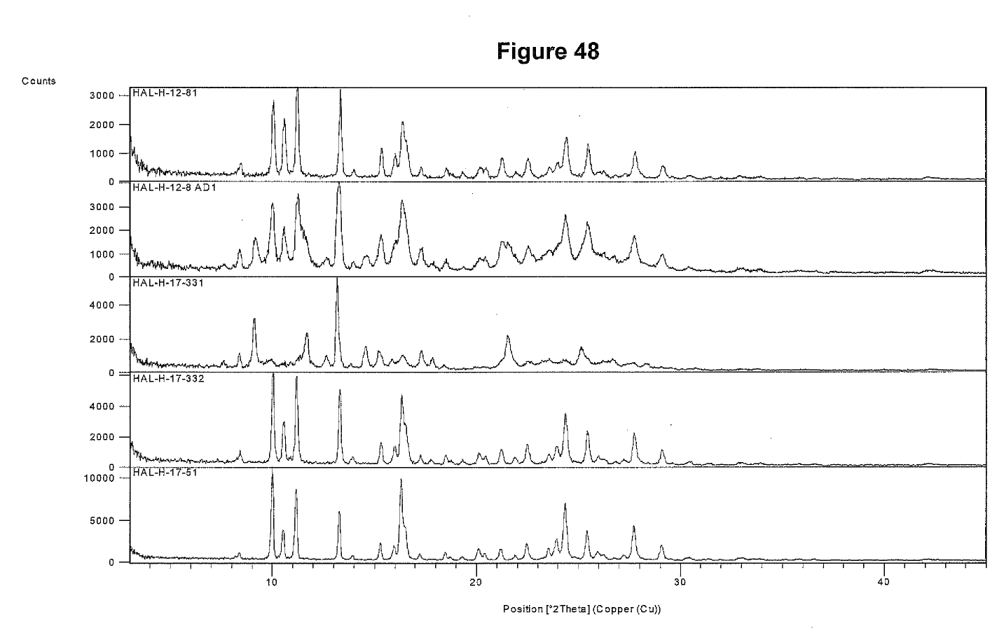

Form C was confirmed to be a monohydrate of OSI-906 by gravimetric moisture sorption analysis. The solid form adsorbed approximately 4.2 wt % water at 30% RH which is consistent with the theoretical water content (4.1 wt %) of a monohydrate of OSI-906 (See FIG. 47). Upon desorption, hysteresis was observed between 25% RH and 5% RH. Loss of water was observed as the humidity was reduced below 15% indicating that Form C is not stable in this environment. XRPD analysis of the solid recovered from the experiment which had been dried at 60° C./0% RH for two hours afforded a diffraction pattern indicative of a mixture of Form C and an unidentified crystalline form (See FIG. 48). Based on these findings, an additional experiment was conducted in an effort to isolate this new crystalline form. An XRPD substrate containing Form C was placed in a desiccator at room temperature. After overnight storage, the slide was analyzed immediately by XRPD upon removal from the environment and brief exposure (<10 min) to the lab humidity (40-50% RH). The resulting diffraction pattern exhibited unique reflections in comparison to all other identified forms and the solid form was designated Form I (See FIG. 48). The sample was retested after an hour of equilibration to the lab conditions and showed conversion to Form C (See FIG. 48).

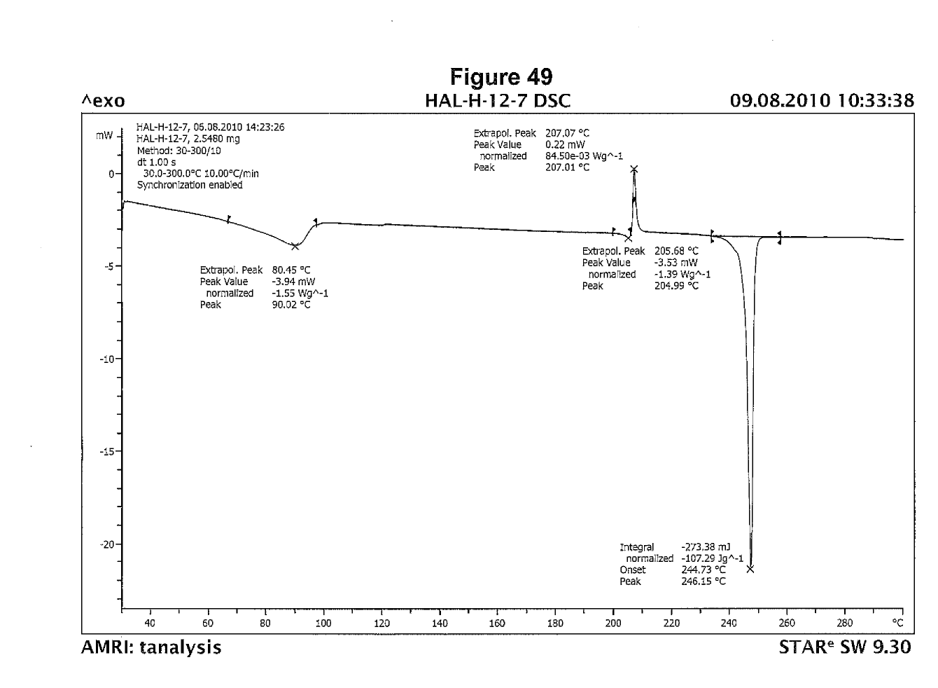

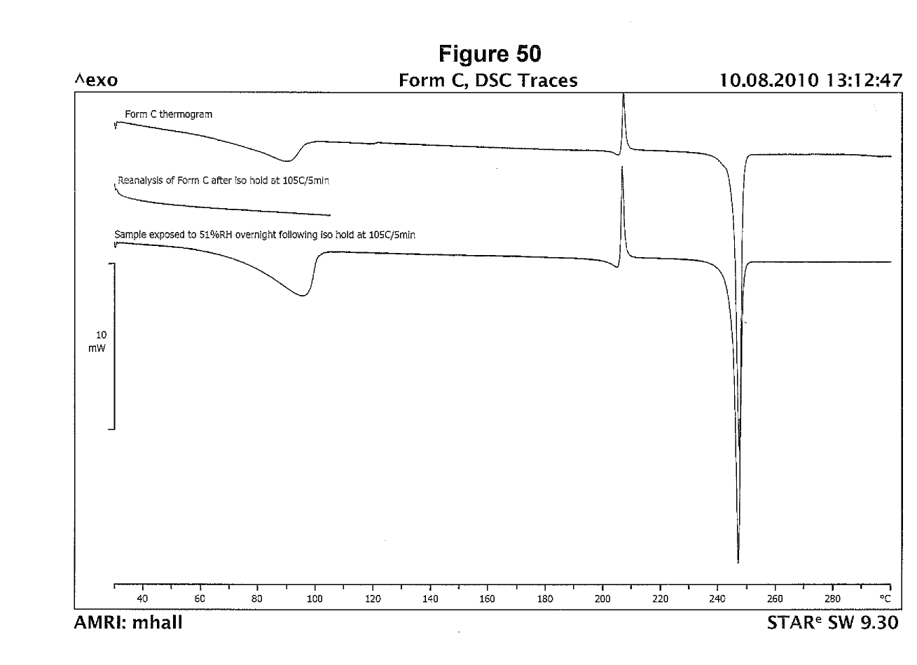

DSC analysis of Form C showed a broad endotherm at 90° C. attributed to loss of water followed by additional events at 205, 207 and melting of Form A 246° C. (See FIG. 49). In an effort to elucidate the additional thermal events, additional DSC experiments were conducted. Form C was held at 105° C. for five minutes, cooled to room temperature and then reheated to the same temperature. As shown in FIG. 50, the initial endotherm at 90° C. was no longer present indicating that water was removed from the sample. XRPD analysis of the recovered material exhibited a diffraction pattern indicative of Form C (See FIG. 48). The isothermal hold experiment was repeated and the sample then exposed to the lab environment (˜40-50% RH) overnight. Reanalysis by DSC showed reappearance of the broad endotherm at 90° C. indicating that the sample re-adsorbed the water upon exposure to the lab (See FIG. 50). These observations are consistent with the results previously presented following storage of Form C under desiccant conditions.

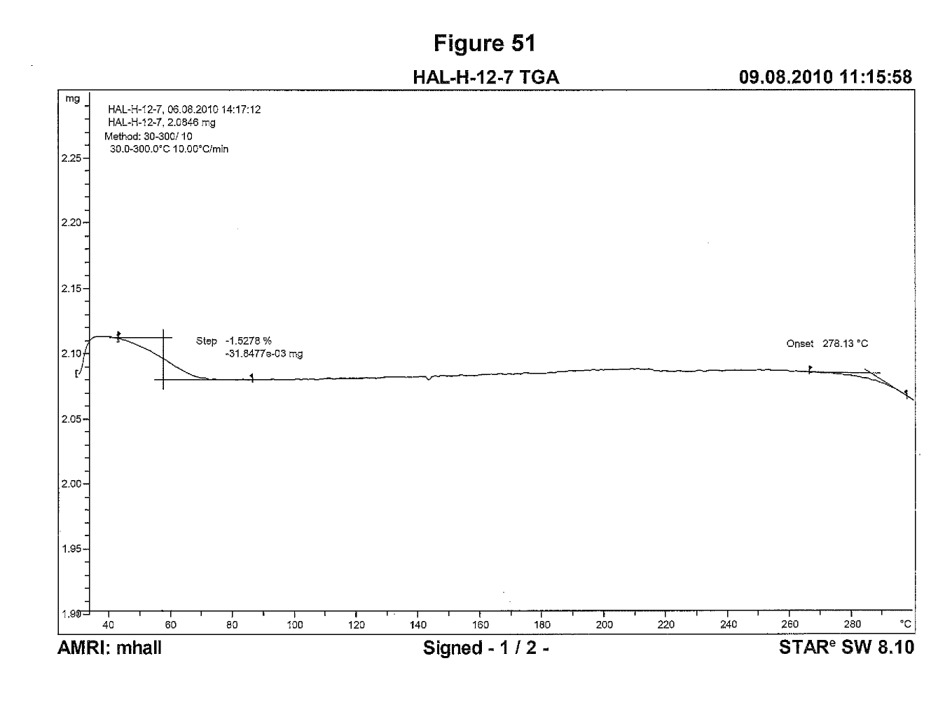

Based on these findings it is likely that the endothermic transition at 205° C. is attributed to melting of Form I followed by re-crystallization at 207° C. to Form A. These results suggest that Forms I and A are montropically related. KF analysis of Form C showed 4.2 wt % water which is consistent with the results obtained from the gravimetric moisture sorption experiment which indicated that the solid form is a monohydrate of OSI-906. Form C exhibited loss of 1.5 wt % water by TGA (See FIG. 51). This result is lower than the value returned from the Karl Fischer analysis likely due to rapid dehydration endured during exposure to elevated temperature and to the TGA nitrogen environment.

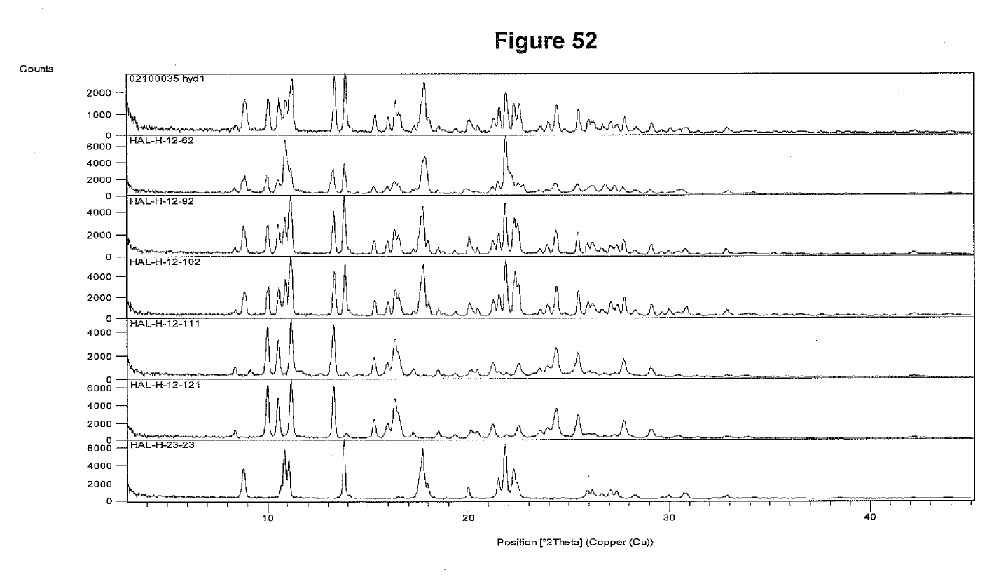

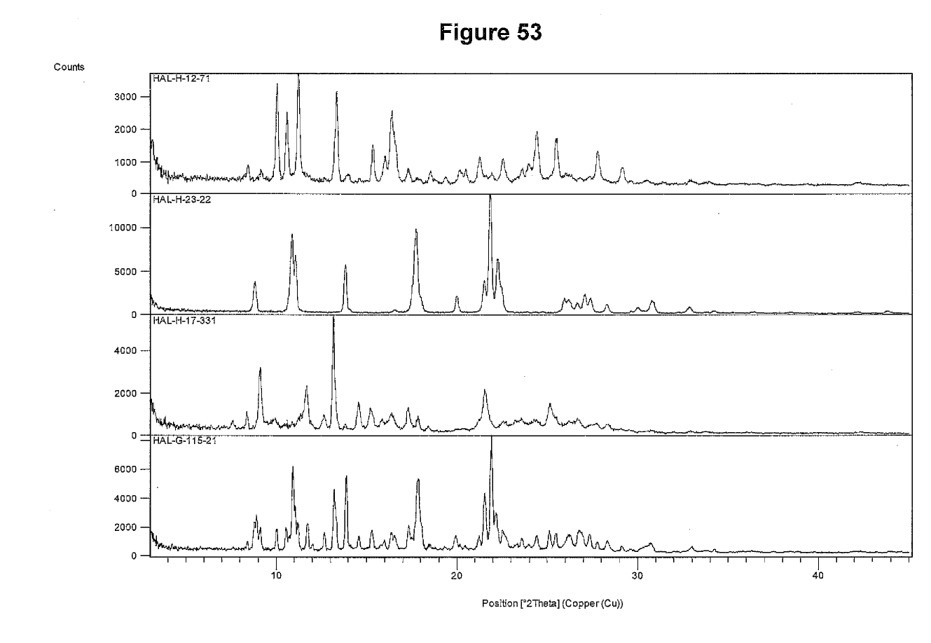

As shown in Table 21, Forms C and D remained a mixture following 1 and seven days of storage at 25° C./60% RH, 40° C./75% RH and under desiccant conditions (See FIG. 52). In contrast to the previous desiccant stability experiment which showed conversion of Form C to Form I, these samples had a much greater residence time in the lab environment (−40-50% RH) likely promoting conversion to the hydrate forms. This conclusion is also supported by an additional experiment with a mixture of Forms C and D, which was conducted in an effort to isolate a sufficient quantity of Form I for a competitive slurry experiment in IPA. As shown in Table 25, after three days of desiccant storage of Forms C and D a mixture of Forms C, D and I was obtained (See FIG. 53). The mixture of forms C+D showed conversion to Form C following one and seven days of storage at elevated drying conditions (See FIG. 52). As demonstrated with previous experiments, it is suspected that the hydrate forms dehydrated to Form I followed by conversion to Form C upon exposure to the lab environment.

TABLE 25

| | Mass | Storage | Duration | XRPD |
|---|---|---|---|---|
| Initial Form | (mg) | Condition | (days) | (Form) |
| C + D | 250 | 80° C. under | 3 | C |
| | | vacuum | | |
| | 69 | Desiccant | | C + D + I |

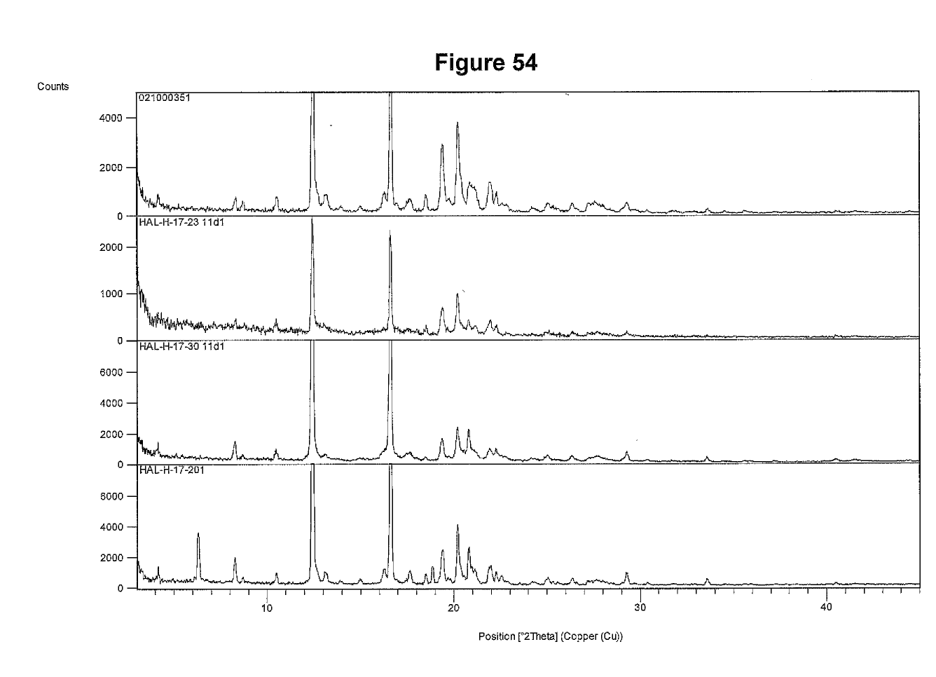

Slurry experiments demonstrated that Form C was stable in water and (80:20) EtOH:Water following prolonged equilibration at ambient and elevated temperature (Table 22). In contrast, Form C showed conversion to Form A in THF and IPA (See FIG. 53). The stability of Form C in EtOH is likely temperature mediated as the crystalline form showed conversion to Forms A or E at elevated temperature while exhibiting stability at ambient conditions (See FIG. 54).

### Thermodynamic Stability (Form D)

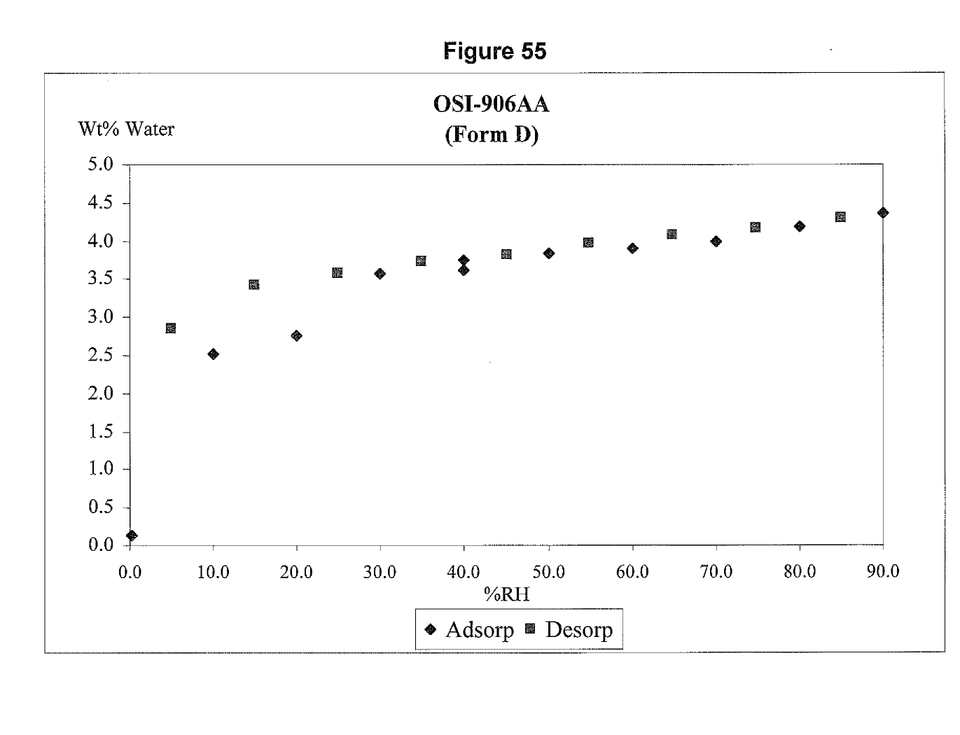

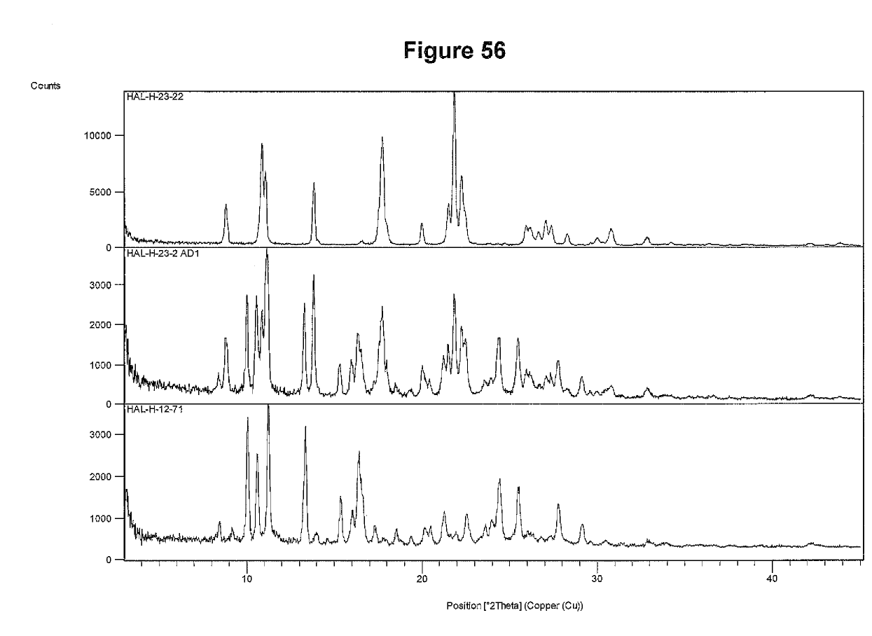

Form D was confirmed to be a monohydrate of OSI-906 by gravimetric moisture sorption analysis. The solid form adsorbed approximately 3.9 wt % water at 60% RH which is comparable to the theoretical water content (4.2 wt %) of a monohydrate of OSI-906 (See FIG. 55). Upon desorption, loss of water was observed as the humidity was reduced below 15% indicating that Form D is not stable in this environment. XRPD analysis of the solid recovered from the experiment which had been dried at 60° C./0% RH for two hours afforded a diffraction pattern indicative of a mixture of Forms C and D (See FIG. 56).

As shown in Table 21, Form D exhibited stability following one and seven days of storage at 25° C./60% RH, 40° C./75% RH and under desiccant conditions. In contrast, Form D showed conversion to Form C at elevated temperature drying conditions (See FIG. 52). Given that Form C is a monohydrate of OSI-906, it is likely that Form D dehydrated to Form I which then converted to Form C upon exposure to the humid lab environment (40-50% RH).

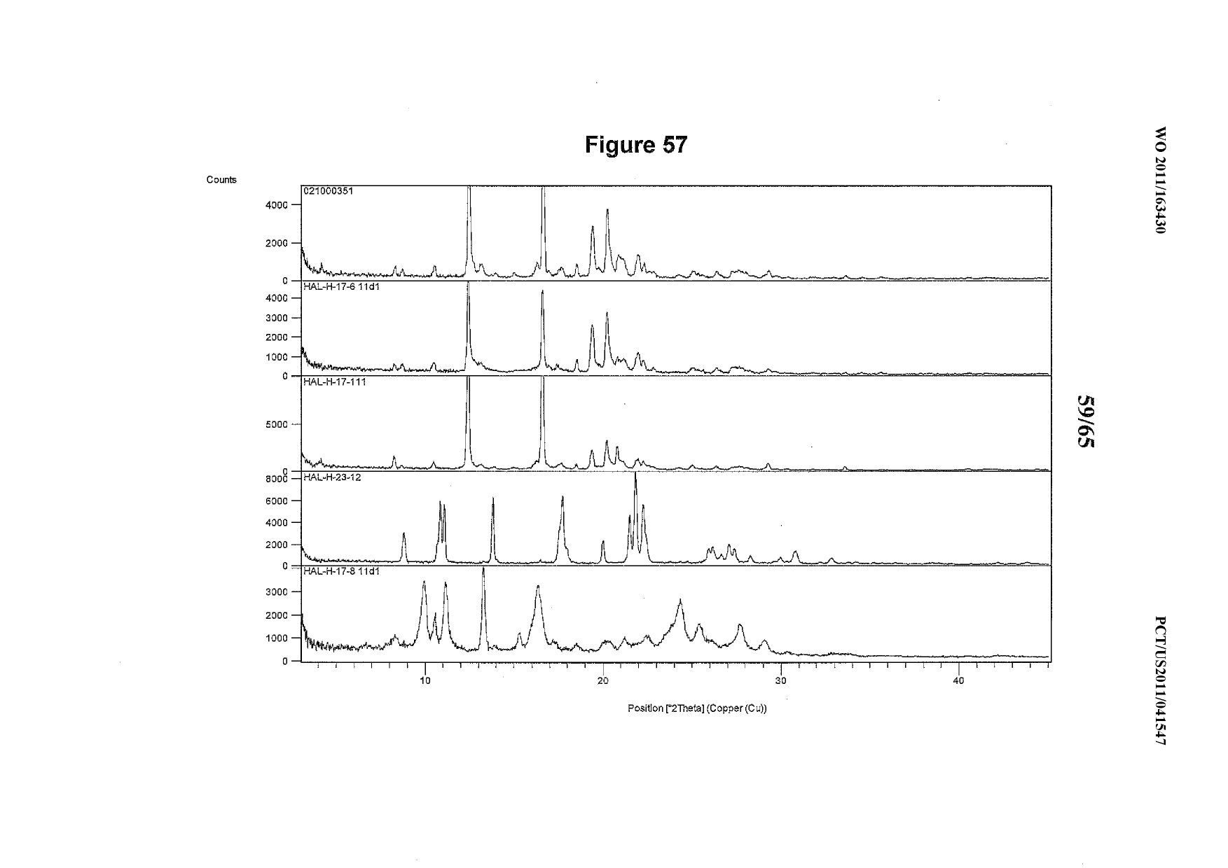

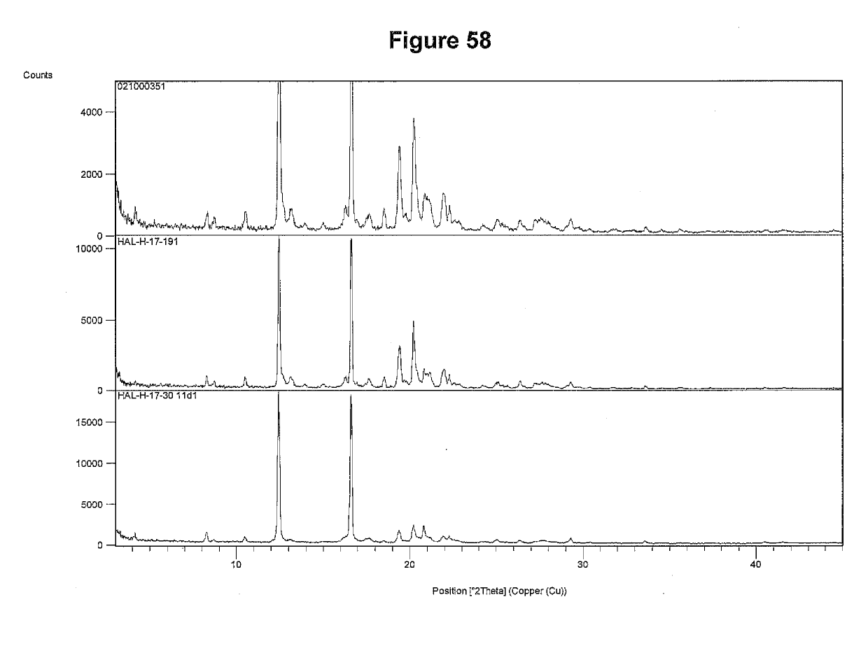

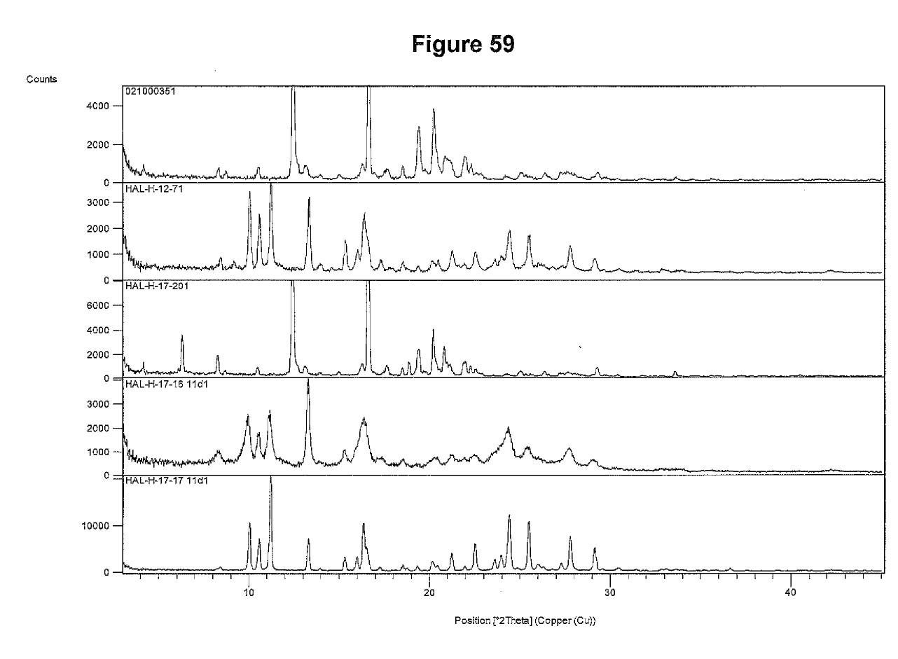

Slurry experiments demonstrated that Form D is stable in water following prolonged equilibration at ambient and elevated temperature (Table 22, FIG. 57). Mixtures of Forms C and D showed no signs of conversion in water and as a result further investigation would be required to determine the most stable hydrate form of OSI-906. Form D showed conversion to Form A in THF and IPA (See FIG. 58). Form D exhibited instability in EtOH converting to either Form A or E at elevated temperature and Form C at ambient temperature (Attachment 39). In (80:20) EtOH:Water, Form D showed conversion to Form C following extended equilibration at elevated or ambient temperature (See FIG. 59).

### Thermal Stress Experiments (Forms B, D, E, and F)

Solids were stressed under different temperature (40° C. or 80° C.) in a vacuum oven for a measured time period. Samples were analyzed after removal from the stress environment as shown in Table 26.

TABLE 26

| Starting | | |
|---|---|---|
| Material | Conditions | XRPD Result |
| Form B | 40° C., vacuum oven, 3 d | Form C |
| Form D | 40° C., vacuum oven, 3 d | Form C |
| Form E | 40° C., vacuum oven, 3 d | Form C |
| Form E | 80° C., vacuum oven, | Form C |
| | overnight | |
| Form F | 40° C., vacuum oven, 3 d | Form C |
| Form F | 80° C., vacuum oven, | Form C |
| | overnight | |
| Form F | 80° C., vacuum oven, | Form C |
| | overnight | |

### Quantitative Determination of Forms A, C and D in OSI-906 by Raman Spectroscopy:

A quantification method for Forms A, C and D in OSI-906 has been developed based on Raman spectroscopy and PLS (partial least squares) regression.

### DEFINITIONS

Accuracy: The accuracy test is used to verify that the Raman method has adequate accuracy for determination of Form C or D in OSI-906 drug substance. The Form C and D concentrations determined by the Raman method are compared with the actual concentrations by gravimetry for synthetic mixtures of Forms A, C and D.

Specificity: The specificity refers to the ability of the quantitation method to assess the concentration of Form C or D in OSI-906 drug substance with presence of Form A.

Limit of Detection (LOD): The smallest concentration of Form C or D in OSI-906 drug substance that can be detected by the quantitation method.

Limit of Quantitation (LOQ): The smallest concentration of Forms C and D in OSI-906 drug substance that can be accurately determined by the quantitation method.

Linearity: The plot of Form C and D concentrations determined by the Raman method against the actual concentrations specified gravimetrically must be linear within the range of the method.

Range: The interval between the lower and upper concentration of Forms C and D that can be determined by the Raman method with a suitable level of accuracy, precision and linearity.

Robustness: The robustness test is to evaluate the performance of the Raman method with variations of the mean sample size.

### Test Method

Reference materials of Forms A, C and D of OSI-906 were used for preparation of the calibration and validation samples.

Analysis Procedure:

Lightly grind approximately 250 mg of sample in a mortar and pestle. Fill a 100 μL aluminum crucible which typically takes approximately 25 mg of ground sample depending on the bulk density of the material (no less than 12 mg should be used for the preparation). Use a spatula to compress the sample and provide a smooth surface. Place the crucible onto the Raman sample stage. Focus microscope and acquire Raman spectrum of the sample. Repeat sample preparation in crucible and acquisition procedure two additional times for a total of three measurements for each ground sample. Save each spectrum in GRAMS SPC file format.

Quantitative Determination of Forms C and D and Calculations:

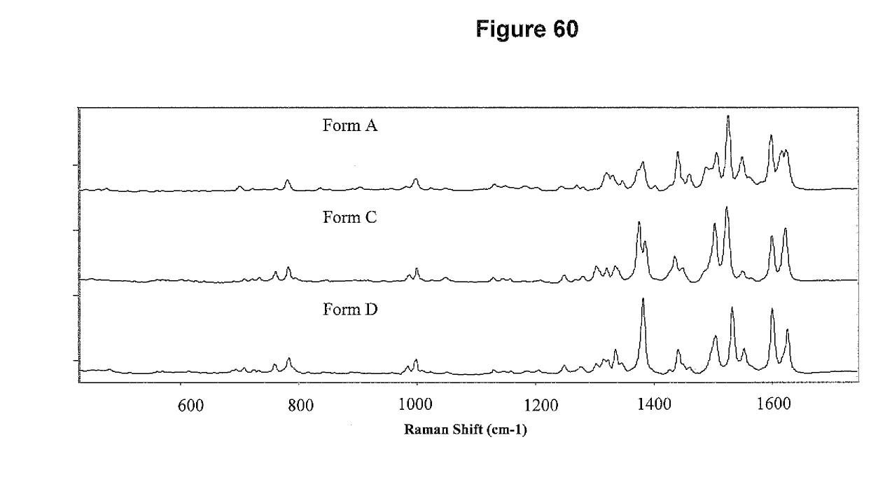

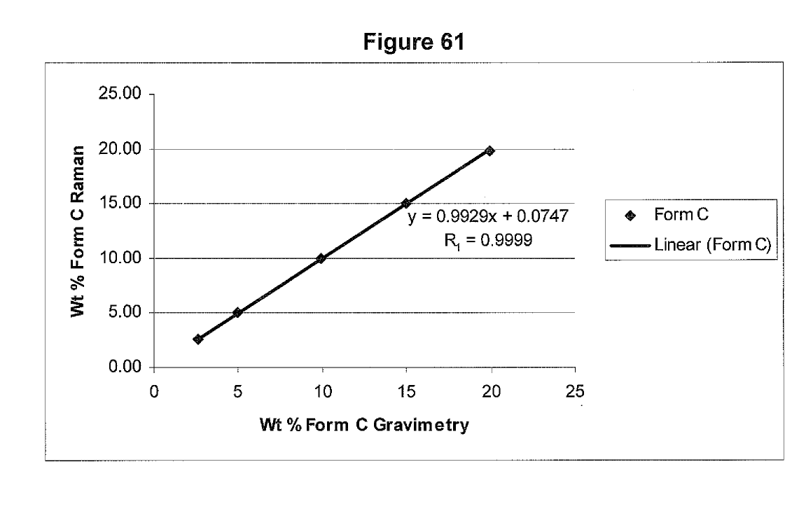

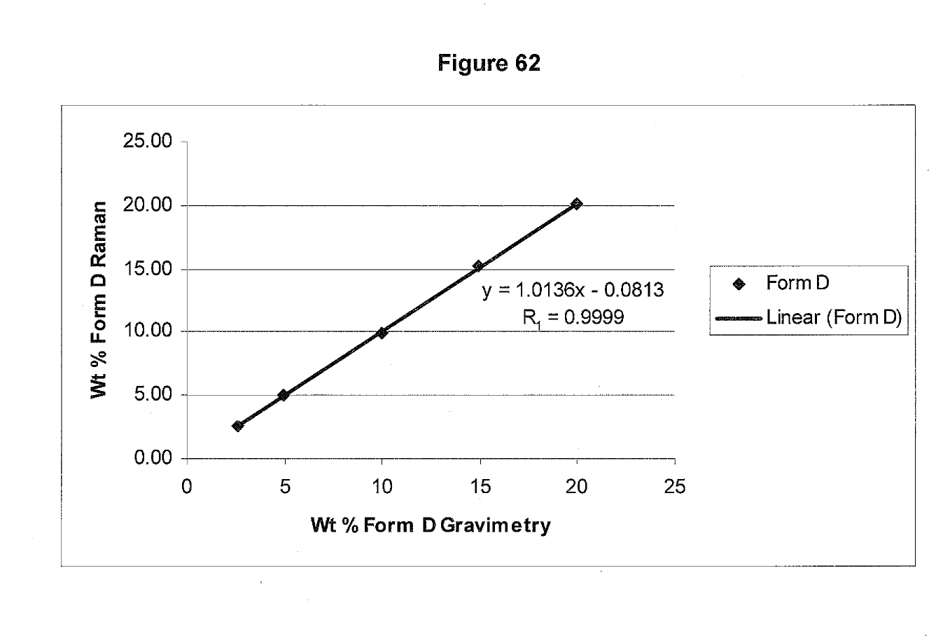

A quantification method for Forms A, C and D in OSI-906 has been developed based on Raman spectroscopy and PLS (partial least squares) regression. The method assumes presence of only Forms A, C and D in the sample. The representative Raman spectra of these three forms are shown in FIG. 60. For the purpose of quantitation, the Raman spectra are pretreated using mean centering normalization. The spectra within the range of 1478-1644 cm⁻¹ were used for PLS regression. TQ analyst software is used for establishing the calibration model (as shown in FIG. 61 and FIG. 62) and quantifying the samples. Weight percentage (wt %) of Forms C and D is determined using the calibration model.

Load the quantitation method using TQ Analyst software for quantifying the three spectra obtained for the sample. Print out the quantitation report for each spectrum. Calculate the average of Forms C and D concentration in wt % for the triplicate measurements.

Report the average wt % of Forms C and D to one decimal place if above LOQ (limit of quantitation, 5 wt %), otherwise report as Form C<LOQ and Form D<LOQ.

### Preparation of Sample Mixtures and Data Analysis

### Sample Preparation Procedure:

The calculated amount of Forms A, C and D was weighed according to the desired wt % of Forms C and D and to a total amount of approximately 250 mg. The samples were mixed in a mortar with the help of a spatula and slightly ground for 5 minutes to obtain consistency and homogeneity. The details of the samples prepared are summarized in Tables 27 and 28.

TABLE 27

| Mass Form A | Mass Form | Mass Form | Wt % | Wt % | Wt % |
|---|---|---|---|---|---|
| (mg) | C (mg) | D (mg) | Form A | Form C | Form D |
| 150.27 | 50.10 | 50.48 | 59.90 | 19.97 | 20.12 |
| 174.65 | 37.33 | 37.73 | 69.94 | 14.95 | 15.11 |
| 200.89 | 24.95 | 24.91 | 80.12 | 9.95 | 9.93 |
| 224.44 | 12.39 | 12.56 | 90.00 | 4.97 | 5.04 |
| 237.47 | 6.53 | 6.39 | 94.84 | 2.61 | 2.55 |

TABLE 28

| Mass Form A | Mass Form | Mass Form | Wt % | Wt % | Wt % |
|---|---|---|---|---|---|
| (mg) | C (mg) | D (mg) | Form A | Form C | Form D |
| 149.73 | 50.38 | 50.38 | 59.77 | 20.11 | 20.11 |
| 175.09 | 37.69 | 38.03 | 69.81 | 15.03 | 15.16 |
| 200.53 | 24.86 | 25.75 | 79.85 | 9.90 | 10.25 |
| 225.77 | 12.68 | 13.09 | 89.75 | 5.04 | 5.20 |
| 237.77 | 6.56 | 6.49 | 94.80 | 2.62 | 2.59 |

The calibration and validation samples were analyzed according to the Test Method to obtain Raman spectra. Quantitative determination of Forms C and Form D was then performed using TQ Analyst software (version 7.1). For the purpose of quantitation, the Raman spectra are pretreated using a quadratic baseline correction based on the region between 1478 and 1654 cm⁻¹ to correct baseline shifts and intensity variation among samples. The Raman spectra within the range of 1478-1654 cm⁻¹ were used for PLS (partial least squares) regression with mean centering normalization.

Acceptance Criteria: <8 wt % calculated as abs[(average Form C or Form D wt % determined)−(actual Form C or Form D wt %)]

Triplicate determinations were performed for each sample prepared according to the Test Method. The average, standard deviation (SD) and relative standard deviation (RSD) of Forms C and D wt % for each sample were calculated and summarized in Tables 29 and 30. The accuracy of method as determined by the maximum difference between the average Form C or D wt % determined and the actual Form C or D wt % for all validation samples is ±1.7 wt %. This is less than 8 wt %, which is the acceptance criteria for the accuracy of the method. The accuracy of the method is thus confirmed.

TABLE 29

| | Actual | Calculated | Average | | | |
|---|---|---|---|---|---|---|
| | Form C | Form C | Form C | SD | RSD | Accuracy¹ |
| Repetition | (wt %) | (wt %) | (wt %) | (wt %) | (%) | (wt %) |
| 1 | 20.11 | 19.09 | 18.43 | 0.7 | 3.9 | 1.7 |
| 2 | | 18.54 | | | | |
| 3 | | 17.67 | | | | |
| 1 | 15.03 | 14.09 | 13.83 | 0.3 | 1.8 | 1.2 |
| 2 | | 13.58 | | | | |
| 3 | | 13.83 | | | | |
| 1 | 9.90 | 9.46 | 9.05 | 0.4 | 4.0 | 0.9 |
| 2 | | 8.79 | | | | |
| 3 | | 8.89 | | | | |
| 1 | 5.04 | 4.82 | 5.05 | 0.2 | 4.7 | 0.0 |
| 2 | | 5.29 | | | | |
| 3 | | 5.05 | | | | |
| 1 | 2.62 | 2.64 | 2.70 | 0.1 | 2.0 | 0.1 |
| 2 | | 2.75 | | | | |
| 3 | | 2.70 | | | | |

¹Accuracy = abs[(average Form C wt % determined) − (actual Form C wt %)]

TABLE 30

| | Actual | Calculated | Average | | | |
|---|---|---|---|---|---|---|
| | Form D | Form D | Form D | SD | RSD | Accuracy¹ |
| Repetition | (wt %) | (wt %) | (wt %) | (wt %) | (%) | (wt %) |
| 1 | 20.11 | 20.74 | 20.15 | 0.6 | 2.8 | 0.0 |
| 2 | | 20.10 | | | | |
| 3 | | 19.60 | | | | |
| 1 | 15.16 | 15.29 | 15.17 | 0.3 | 2.2 | 0.0 |
| 2 | | 14.79 | | | | |
| 3 | | 15.43 | | | | |
| 1 | 10.25 | 10.86 | 10.52 | 0.3 | 3.0 | 0.3 |
| 2 | | 10.44 | | | | |
| 3 | | 10.25 | | | | |
| 1 | 5.20 | 6.14 | 6.29 | 0.2 | 2.9 | 1.1 |
| 2 | | 6.49 | | | | |
| 3 | | 6.23 | | | | |
| 1 | 2.59 | 3.89 | 3.89 | 0.0 | 1.0 | 1.3 |
| 2 | | 3.93 | | | | |
| 3 | | 3.85 | | | | |

¹Accuracy = abs[(average Form D wt % determined) − (actual Form D wt %)]

### Acceptance Criteria: <8 wt %

According to the results obtained, the accuracy of the method was determined to be ±1.7 wt %. Based on these observations, the LOQ determined as the lowest concentration of Forms C and D in samples with acceptable precision and accuracy is 5 wt %, which is less than the acceptance criteria of 8 wt %, thus the LOQ of the method is acceptable. As detection of the method is via quantitation, the LOD of the quantitation method was established as the same as LOQ, i.e., 5 wt %. This is less than 8 wt %, thus the LOD of the method is acceptable.

Acceptance Criteria:
R₁≧0.95 where R₁ is the correlation coefficient for calibration samples
R₂≧0.95 where R₂ is the correlation coefficient for combined validation samples and calibration samples

The average wt % of individual Forms C and D determined by Raman was plotted against the actual wt % of Forms C and D specified gravimetrically for the calibration samples, as shown in FIGS. 61 and 62. Linear regression was performed and is shown on the plot. The correlation coefficient (R₁) for the Form C calibration samples was determined to be 0.9999, greater than 0.95 set as the acceptance criteria for linearity. The slope and the y-intercept of the regression line are 0.9929 and 0.0747, respectively. The correlation coefficient (R₁) for the Form D calibration samples was determined to be 0.9999, greater than 0.95 set as the acceptance criteria for linearity. The slope and the y-intercept of the regression line are 1.0136 and −0.0813, respectively. The acceptance criteria of R₁>0.95 was met for both regression lines.

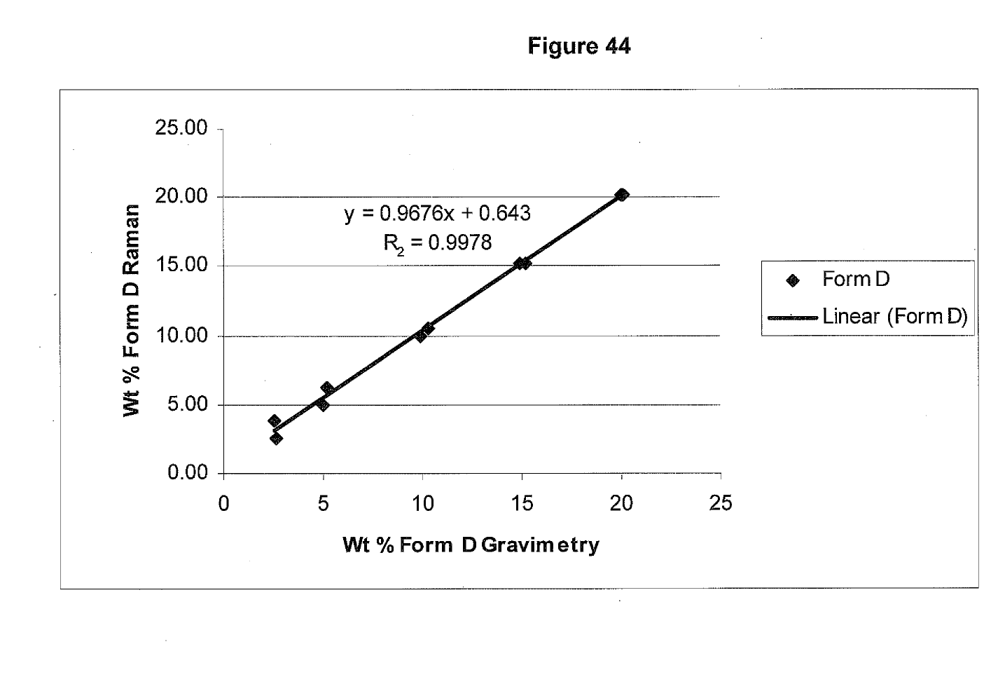

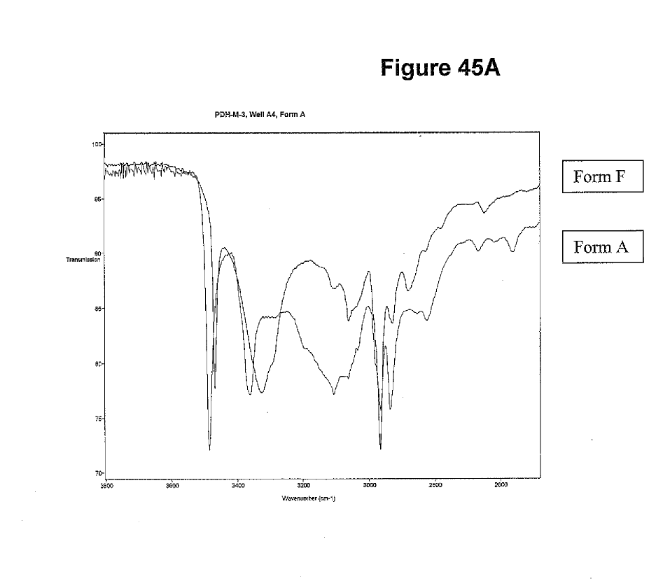

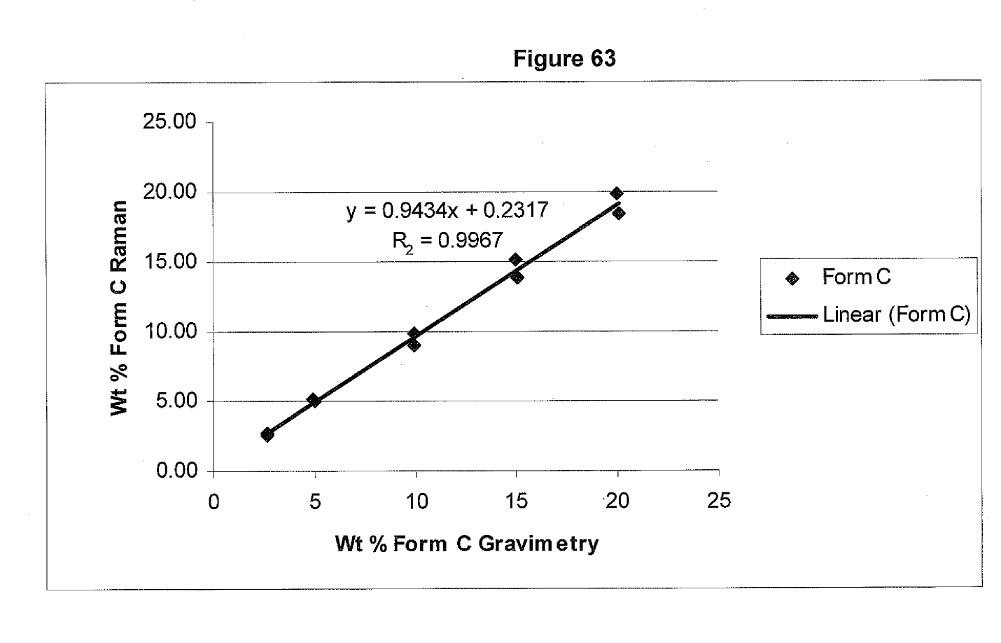

In addition to determining the linearity of the method using calibration samples, linearity was evaluated using the combined results for the validation samples and the calibration samples per the requirement of the validation protocol. The average wt % of Forms C and D determined by Raman was plotted against the actual wt % of Forms C and D specified gravimetrically for the validation samples and the calibration samples, as shown in FIGS. 63 and 44. Linear regression was performed and is shown on the plot. The correlation coefficient (R₂) was determined to be 0.9967 for the Form C samples. The y-intercept and the slope of the regression line are 0.9434 and 0.2317, respectively. The correlation coefficient (R₂) was determined to be 0.9978 for the Form D samples. The y-intercept and the slope of the regression line are 0.9676 and 0.643, respectively. The acceptance criteria of R₂>0.95 was met indicating the method is linear for determination of Form C and Form D in OSI-906 drug substance in the presence of Form A.

The range of the quantitation method is established as between the LOQ and the highest concentration of Forms C or D used in the validation samples with acceptable precision and accuracy. Thus the validated range of the method is between 5 and 20 wt %.

In some aspects, there is provided a pharmaceutical composition comprising the polymorph of any one of Forms A-H, formulated with or without one or more pharmaceutically acceptable carriers.

In some aspects, there is provided a method of treating cancer mediated at least in part by IR and/or IGF-1R comprising administering to a patient in need thereof a therapeutically effective amount of composition of crystalline polymorph of any one of Forms A-H.

In some aspects, there is provided a method of treating sarcoma, fibrosarcoma, osteoma, melanoma, retinoblastoma, rhabdomyosarcoma, neuroblastoma, teratocarcinoma, hematopoietic malignancy, malignant ascites, lung cancer, gastric cancer, head and neck cancer, bladder cancer, prostate cancer, esophageal squamous cell carcinoma, anaplastic large cell lymphoma, inflammatory myofibroblastic tumor, or glioblastoma with a therapeutically effective amount of composition of crystalline polymorph of any one of Forms A-H.

In further aspects, there is provided a method of treating adrenocortical carcinoma, colorectal cancer, non-small cell lung cancer, breast cancer, pancreatic cancer, ovarian cancer, hepatocellular carcinoma, or renal cancer with a therapeutically effective amount of composition of crystalline polymorph of any one of Forms A-H.

### Compositions

The invention provides pharmaceutical compositions of OSI-906 polymorphic Forms A-H formulated for a desired mode of administration with or without one or more pharmaceutically acceptable and useful carriers. The compounds can also be included in pharmaceutical compositions in combination with one or more other therapeutically active compounds.

The pharmaceutical compositions of the present invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight or more.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Compositions containing a compound described by Formula I, or pharmaceutically acceptable salts thereof, may also be prepared in powder or liquid concentrate form.

### Biological Activity and Uses

Further still, the invention provides for methods of treating cancer with an IGF-1R inhibitor polymorphic Forms of OSI-906, which includes unsolvated Form A, hydrated Forms B-E and solvated Forms F and G.

The efficacy of OSI-906 as an inhibitor of insulin-like growth factor-I receptor (IGF-IR) was demonstrated and confirmed by a number of pharmacological in vitro assays. The assays and their respective methods can be carried out with the compounds according to the invention. Activity possessed by OSI-906 has been demonstrated in vivo. See, e.g., Future Med. Chem., 2009, 1(6), 1153-1171.

US 2006/0235031 (published Oct. 19, 2006) describes a class of bicyclic ring substituted protein kinase inhibitors, including Example 31 thereof, which corresponds to the IGF-1R inhibitor known as OSI-906. OSI-906 is in clinical development in various tumor types.

The present invention includes a method of inhibiting protein kinase activity comprising administering a compound of Formula I or a pharmaceutically acceptable salt thereof.

The present invention includes a method of inhibiting IGF-1R activity comprising administering a compound of Formula I or a pharmaceutically acceptable salt thereof.

The present invention includes a method of inhibiting protein kinase activity wherein the activity of said protein kinase affects hyperproliferative disorders comprising administering a compound of Formula I or a pharmaceutically acceptable salt thereof.

The present invention includes a method of inhibiting protein kinase activity wherein the activity of said protein kinase influences angiogenesis, vascular permeability, immune response, cellular apoptosis, tumor growth, or inflammation comprising administering a compound of Formula I or a pharmaceutically acceptable salt thereof.

The present invention includes a method of treating a patient having a condition which is mediated by protein kinase activity, said method comprising administering to the patient a therapeutically effective amount of a compound of Formula I or a pharmaceutically acceptable salt thereof.

The present invention includes a method of treating a patient having a condition which is mediated by IGF-1R activity, said method comprising administering to the patient a therapeutically effective amount of a compound of Formula I or a pharmaceutically acceptable salt thereof.

The present invention includes a method of treating a patient having a condition which is mediated by protein kinase activity wherein the condition mediated by protein kinase activity is cancer, said method comprising administering to the patient a therapeutically effective amount of a compound of Formula I or a pharmaceutically acceptable salt thereof.

In some aspects, the invention includes a method of treating a cancer, such as those above, which is mediated at least in part by IR and/or IGF-1R comprising administering to a mammal in need thereof a therapeutically effective amount of a compound or salt of the invention. In some aspects thereof, the cancer is mediated at least in part by amplified IGF-1R. In some aspects thereof, the compound is a dual IGF-1R and IR inhibitor, and can be a selective inhibitor.

The compounds of Formula I of the present invention are useful in the treatment of a variety of cancers, including, but not limited to, solid tumor, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some aspects, the above methods are used to treat one or more of bladder, colorectal, nonsmall cell lung, breast, or pancreatic cancer. In some aspects, the above methods are used to treat one or more of ovarian, gastric, head and neck, prostate, hepatocellular, renal, glioma, glioma, or sarcoma cancer.

In some aspects, the invention includes a method, including the above methods, wherein the compound is used to inhibit EMT. IGF-1R is widely expressed in human epithelial cancers. The role of IGF-1R is critical with colorectal, NSCLC, and ovarian cancers, whereby tumors may drive their growth and survival through over-expression of autocrine IGF-II. Development of prostate, breast and colorectal cancer with respect to expression of IGF-1 has been widely studied. Hence, IGF-1R represents an important therapeutic target for the treatment of cancer when employed to inhibit EMT. OSI-906 is expected to potentiate the antitumor activity of a broad range of tumor types through IGF-1R as well as other receptors.

The present invention includes a formulation intended for the preferred oral administration to humans.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In some aspects, the invention includes a method of treating cancer comprising administering to a mammal in need thereof a therapeutically effective amount of a compound or salt of the invention, wherein at least one additional active anti-cancer agent is used as part of the method. The present invention includes a method for treating tumors or tumor metastases in a patient, comprising administering to said patient simultaneously or sequentially a therapeutically effective amount of an EGFR kinase inhibitor and the compound of Formula I, additionally comprising one or more other anti-cancer agents. The present invention includes a method for treating tumors or tumor metastases in a patient, comprising administering to said patient simultaneously or sequentially a therapeutically effective amount of the EGFR kinase inhibitor erlotinib and the compound of Formula I, additionally comprising one or more other anti cancer agents.

The present invention includes a method for treating tumors or tumor metastases in a patient, comprising administering to said patient simultaneously or sequentially a therapeutically effective amount of an EGFR kinase inhibitor and the compound of Formula I, additionally comprising one or more other anti-cancer agents, wherein the other anti-cancer agents are one or more agents selected from an alkylating agent, cyclophosphamide, chlorambucil, cisplatin, busulfan, melphalan, carmustine, streptozotocin, triethylenemelamine, mitomycin C, an anti-metabolite, methotrexate, etoposide, 6-mercaptopurine, 6-thiocguanine, cytarabine, 5-fluorouracil, raltitrexed, capecitabine, dacarbazine, an antibiotic, actinomycin D, doxorubicin, daunorubicin, bleomycin, mithramycin, an alkaloid, vinblastine, paclitaxel, a glucocorticoid, dexamethasone, a corticosteroid, prednisone, a nucleoside enzyme inhibitors, hydroxyurea, an amino acid depleting enzyme, asparaginase, folinicacid, leucovorin, and a folic acid derivative.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

Further, the compounds may be amorphous or may exist or be prepared in various crystal forms or polymorphs, including solvates and hydrates. The invention includes any such forms provided herein, at any purity level. A recitation of a compound per se means the compound regardless of any unspecified stereochemistry, physical form and whether or not associated with solvent or water.

The compounds of the invention are not limited to those containing all of their atoms in their natural isotopic abundance. Rather, a recitation of a compound or an atom within a compound includes isotopologs, i.e., species wherein an atom or compound varies only with respect to isotopic enrichment and/or in the position of isotopic enrichment. For example, in some cases it may be desirable to enrich one or more hydrogen atoms with deuterium (D) or to enrich carbon with ¹³O.

When a tautomer of the compound of Formula I exists, the compound of Formula I of the present invention includes any possible tautomers and pharmaceutically acceptable salts thereof, and mixtures thereof, except where specifically stated otherwise.

The invention also encompasses a pharmaceutical composition that is comprised of a compound of Formula I in combination with a pharmaceutically acceptable carrier.

Preferably the composition is comprised of a pharmaceutically acceptable carrier and a non-toxic therapeutically effective amount of a compound of Formula I as described above (or a pharmaceutically acceptable salt thereof).

Moreover, within this preferred embodiment, the invention encompasses a pharmaceutical composition for the treatment of disease by inhibiting kinases, comprising a pharmaceutically acceptable carrier and a non-toxic therapeutically effective amount of compound of Formula I as described above (or a pharmaceutically acceptable salt thereof).

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids. Particularly preferred are formic and hydrochloric acid.

### General Definitions and Abbreviations

Unless otherwise specified, terms used herein shall have the same meaning as commonly understood by one of ordinary skill in the art, as per the invention. Furthermore, while equivalent methods and materials can be used to practice the invention, the preferred methods and materials are described.

Each variable definition above includes any subset thereof and the compounds of Formula I include any combination of such variables or variable subsets.

In some aspects, the invention includes any of the compound examples herein and pharmaceutically acceptable salts thereof.

The invention includes the compounds and salts thereof, and their physical forms, preparation of the compounds, useful intermediates, and pharmaceutical compositions and formulations thereof.

The term “XRPD” refers to X-ray powder diffraction.

The term “RH” refers to relative humidity.

The term “isolating” refers to indicate separation or collection or recovery of the compound of the invention being isolated in the specified form.

The phrase “preparing a solution” refers to obtaining a solution of a substance in a solvent in any manner. The phrase also includes a partial solution or slurry.

The term “stable” refers to the tendency of a compound to remain substantially in the same physical form for at least one month, preferably six months, more preferably at least one year or at least three years under ambient conditions (20° C./60% RH).

The phrase “substantially in the same physical form” refers to at least 70%, preferably 80%, and more preferably 90% of the crystalline form remains and more preferably 98% of the crystalline form remains.

The term “form” refers to a novel crystalline form that can be distinguished by one of skill in the art from other crystalline forms based on the details provided herein.

The phrase “substantially free” refers to at least less than 5%, preferably less than 2% as weight %.

The term “slurry” refers to solutions prepared by adding enough solids to a given solvent so that excess solids were present.

The term “polar solvent” refers to 1,4-dioxane, dichloromethane, tetrahydrofuran, ethyl acetate, acetone, dimethylformamide, acetonitrile, nitromethane, dimethyl sulfoxide, formic acid, n-butanol, t-butanol, 2-butanol, isopropanol, n-propanol, ethanol, methanol, acetic acid, water and solvents with a dielectric constant greater than about 15.

The following abbreviations are used:
B/R birefringence
ext. extinction
min. minute(s)
h hour(s)
d day(s)
RT or rt room temperature
t_{R} retention time
L liter
mL milliliter
mmol millimole
pmol micromole
equiv. or eq. equivalents
NMR nuclear magnetic resonance
MDP(S) mass-directed HPLC purification (system)
LC/MS liquid chromatography mass spectrometry
HPLC high performance liquid chromatography
TLC thin layer chromatography
CDCl₃ deuterated chloroform
CD₃OD or MeoD deuterated methanol
DMSO-d₆ deuterated dimethylsulfoxide
LDA lithium diisopropylamide
DCM dichloromethane
THF tetrahydrofuran
EtOAc ethyl acetate
MeCN acetonitrile
DMSO dimethylsulfoxide
Boc tert-butyloxycarbonyl
DME 1,2-dimethoxyethane
DMF N,N-dimethylformamide
DIPEA diisopropylethylamine
PS-DIEA polymer-supported diisopropylethylamine
PS—PPh₃-Pd polymer-supported Pd(PPh₃)₄
EDC 1-(3-dimethylaminopropyl)-3-ethylcarbodiimide
HOBt 1-hydroxybenzotriazole
DMAP 4-dimethylaminopyridine
TBTU O-(benzotriazol-1-yl)-N,N,N′,N′-tetramethyluronium tetrafluoroborate
TEMPO 2,2,6,6-tetramethylpiperidine-1-oxyl
TFA trifluoroacetic acid

What is claimed is:

1. Crystalline polymorph Form A of OSI-906.

2. The polymorph Form A of claim 1, which exhibits an X-ray diffraction pattern with characteristic peaks substantially as set forth in Table 1, an X-ray diffraction pattern essentially resembling that of FIG. 2, a TGA profile substantially resembling FIG. 17, or a DSC thermogram substantially resembling that of FIG. 16.

3. The polymorph Form A of claim 1, which exhibits an X-ray diffraction pattern comprising peaks (°2θ) at about 12.4, 12.6, 16.6, 18.5, 19.4, 20.2, and 22.

4. (canceled)

5. The polymorph of claim 1, present as a material comprising at least about 98% by weight Form A based on the total amount of OSI-906.

6. The polymorph of claim 5, which is present as a material that is substantially free of amorphous OSI-906, OSI-906 hydrates, and OSI-906 solvates.

7. The polymorph of claim 6, which is substantially free of solvent.

8. The polymorph of claim 1, which is prepared by a process comprising: (a) preparing a slurry of OSI-906 in an alcohol; (b) heating the slurry; and (c) isolating crystalline Form A.

9. The polymorph of claim 8, wherein the preparing a slurry in (a) further comprises adjusting pH to about 5 and the alcohol in (a) comprises isopropanol, n-propanol, n-butanol, sec-butanol, t-butanol, or iso-butanol.

10. (canceled)

11. Crystalline polymorph Form B of OSI-906.

12. The polymorph Form B of OSI-906 of claim 11, which exhibits an X-ray diffraction pattern with characteristic peaks as set forth in Table 3, an X-ray diffraction pattern substantially resembling that of FIG. 3, a DSC thermogram substantially resembling that of FIG. 18, a TGA signal substantially resembling that of FIG. 19, or a ¹H NMR spectrum in DMSO-d₆ substantially resembling that of FIG. 31.

13. The polymorph Form B of claim 11, which exhibits an X-ray diffraction pattern comprising peaks (°2θ) at about 10.1, 10.6, 11.2, 13.3, 15.3, 16.3, 21.8, 22.3, 22.4, 24.4, and 27.8.

14. Crystalline polymorph Form C of OSI-906.

15. The polymorph Form C of claim 14, which exhibits an X-ray diffraction pattern with characteristic peaks as set forth in Table 5, an X-ray diffraction pattern substantially resembling that of FIG. 4, a DSC thermogram substantially resembling that of FIG. 20, or a TGA signal substantially resembling that of FIG. 21.

16. The polymorph Form C of claim 14, which exhibits an X-ray diffraction pattern comprising peaks (°2θ) at about 10.6, 11.2, 13.3, 15.3, 21.2, 24.3, and 25.5.

17. The polymorph Form C of claim 14, which is present as a material comprising at least about 95% or more by weight Form C based on the total amount of OSI-906.

18. The polymorph of claim 17, which is present as a material that is substantially free of amorphous OSI-906, OSI-906 hydrates, and OSI-906 solvated, other than polymorph Form C.

19. The polymorph of claim 18, which is prepared by a process comprising: (a) preparing a solution of OSI-906 in an alcohol; (b) heating the solution; and (c) isolating crystalline Form C.

20-28. (canceled)
